(19) United States
(12) Patent Application Publication
(10) Pub. No.: US 2007/0142226 A1
(43) Pub. Date: Jun. 21, 2007

(54) Method and agents for improving plant productivity involving endophytic actinomycetes and metabolites thereof

(76) Inventor: Christopher Milton Mathew Franco, PAYNEHAM (AU)

Correspondence Address: SCULLY SCOTT MURPHY & PRESSER, PC, 400 GARDEN CITY PLAZA, SUITE 300, GARDEN CITY, NY, 11530, US

(21) Appl. No.: 10/563,637

(22) PCT Filed: Jul. 7, 2004
(86) PCT No.: PCT/AU04/00914

§ 371 Date: Jun. 12, 2006

Related U.S. Application Data
(60) Provisional application No. 60/485,241, filed on Jul. 7, 2003.
(60) Provisional application No. 60/504,703, filed on Sep. 22, 2003.

(51) Int. Cl. A01N 63/00; C12N 1/20

(52) U.S. Cl. 504/117; 435/252.2

(57) ABSTRACT

The present invention relates to a method for improving plant productivity, and in particular crop yields, via the introduction of an endophytic microorganism to the subject plant. More particularly, the present invention is directed to a method for improving cereal crop productivity via the introduction of an endophytic actinomycete to the subject crop. The method of the present invention facilitates the improvement of crop productivity, such as increasing germination, by, inter alia, providing the subject plant with disease bio-control capabilities and up-regulating plant growth promoting activities. The present invention is also directed to novel endophytic microorganisms and uses thereof.

### FIELD OF THE INVENTION

The present invention relates to a method for improving plant productivity, and in particular crop yields, via the introduction of an endophytic microorganism to the subject plant. More particularly, the present invention is directed to a method for improving cereal crop productivity via the introduction of an endophytic actinomycete to the subject crop. The method of the present invention facilitates the improvement of crop productivity, such as increasing germination, by, inter alia, providing the subject plant with disease bio-control capabilities and up-regulating plant growth promoting activities. The present invention is also directed to novel endophytic microorganisms and uses thereof.

### BACKGROUND OF THE INVENTION

Bibliographic details of the publications referred to by author in this specification are collected alphabetically at the end of the description.

The reference to any prior art in this specification is not, and should not be taken as, an acknowledgment or any form of suggestion that that prior art forms part of the common general knowledge in Australia.

The wheat crop in Australia is subject to attack by many pests and diseases which reduce the potential grain yields. The major cause of yield reductions is the fungal disease “take all” caused by a major fungal pathogen of wheat crops Gaeumannomyces graminis var. tritici (Ggt) followed by Rhizoctonia. There are currently no reliable means to control these pathogens either by biological or chemical means. In addition to controlling such pathogens, the importance of the agricultural industry in Australia necessitates optimal crop productivity. Accordingly, there is an ongoing need to develop new techniques directed to improving crop productivity in terms of facilitating growth promotion and/or controlling the adverse activity of crop pathogens.

Endophytes are plant associated microorganisms obtained from surface-sterilised plant tissue. It is thought that they inhabit and coexist with the innermost of cells of plants. They are found in the cortex and vascular systems of plant roots and are present in leaves, stems and seeds. Due to their location within the plant, these organisms are free from competition with general microflora in the soil and are protected to a large extent from environmental stresses. Agriculturally, this type of relationship can be put to practical use since metabolites produced by some bacteria may exhibit plant growth promotion and/or pathogen control properties and may induce systemic resistance in plants.

In work leading up to the present invention, the inventors have determined that the microflora of some wheat isolates differs significantly from the microflora commonly found in soil and, surprisingly, even in other wheat isolates. In particular, the inventors have determined that some species of actinomycetes can exist as endophytes in wheat plants and, moreover, contribute to improved productivity of the host plant by virtue of exhibiting functional activities such as pathogen antagonism and production of growth promotion metabolites. In a related aspect, the inventors have further identified a number of novel species of wheat plant endophytic actinomycetes which, inter alia, can provide these benefits. The surprising elucidation of both the endophytic existence of several actinomycete species which were previously thought only to exist in the rhizosphere and the identification of novel actinomycete species in wheat plants, together with the yet more unexpected determination that only some of these wheat plant endophytic actinomycetes also function as modulators of improved plant productivity has now facilitated the development of methodology for improving plant productivity, in particular cereal crop productivity, based on introducing to a plant the subject actinomycete.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The subject specification contains nucleotide and amino acid sequence information prepared using the programme PatentIn Version 3.1, presented herein after the bibliography. Each nucleotide sequence is identified in the sequence listing by the numeric indicator <210> followed by the sequence identifier (eg. <210>1, <210>2, etc). The length, type of sequence, (DNA, protein (PRT), etc) and source organism for each nucleotide or amino acid sequence are indicated by information provided in the numeric indicator fields <211>, <212> and <213>, respectively. Nucleotide and amino acid sequences referred to in the specification are defined by the information provided in numeric indicator field <400> followed by the sequence identifier (eg. <400>1, <400>2, etc).

One aspect of the present invention is directed to a method of improving plant productivity said method comprising introducing into said plant or propagation material thereof:
(i) an effective number of cereal plant-derived endophytic actinomycetes or variants, mutants or homologues thereof, which actinomycetes facilitate induction of at least one characteristic related to improved productivity; and/or
(ii) an effective amount of one or more metabolites derived from the actinomycetes of (i) or derivative, homologue, analogue, chemical equivalent or mimetic thereof;
   for a time and under conditions sufficient to induce, in the subject plant, said characteristic.

Another aspect of the present invention provides a method of improving cereal plant productivity said method comprising introducing into said cereal plant or propagation material thereof:
(i) an effective number of cereal plant-derived endophytic actinomycetes or variants, mutants or homologues thereof; which actinomycetes facilitate induction of at least one characteristic related to improved productivity; and/or
(ii) an effective amount of one or more metabolites derived from the actinomycetes of (i) or derivative, homologue, analogue, chemical equivalent or mimetic thereof;
   for a time and under conditions sufficient to induce, in the subject cereal plant, said characteristic.

Still another aspect of the present invention provides a method of improving plant productivity said method comprising introducing into said plant or propagation material thereof:
(i) an effective number of cereal plant-derived endophytic actinomycetes or variants, mutants or homologues thereof; which actinomycetes facilitate induction of at least one characteristic related to improved productivity; and/or
(ii) an effective amount of one or more metabolites derived from the actinomycetes of (i) or derivative, homologue, analogue, chemical equivalent or mimetic thereof;
   for a time and under conditions sufficient to induce, in the subject plant, growth promotion and/or bio-control activity.

Yet still another aspect of the present invention provides a method of improving cereal plant productivity said method comprising introducing into said plant or propagation material thereof:
(i) an effective number of endophytic actinomycetes of the genus Microbispora, Streptomyces, Micromonospora, Streptosporangiacae, Nocardiodes, Tsukamurella or Streptosporangium or variants, mutants or homologues thereof; and/or
(ii) an effective amount of one or more metabolites derived from the actinomycetes of (i) or derivative, homologue, analogue, chemical equivalent or mimetic thereof;
   for a time and under conditions sufficient to induce, in the subject cereal plant, said characteristic.

Still yet another aspect of the present invention provides a method of improving plant productivity said method comprising introducing into said plant or propagation material thereof:
(i) an effective number of cereal plant-derived endophytic actinomycetes or variants, mutants or homologues thereof; which actinomycetes facilitate induction of at least one characteristic related to improved productivity; and/or
(ii) an effective amount of one or more metabolites derived from the actinomycetes of (i) or derivative, homologue, analogue, chemical equivalent or mimetic thereof;
   for a time and under conditions sufficient to induce, in the subject cereal plant, said characteristic, wherein said actinomycete is selected from the list of:
(a) An actinomycete characterised either by a nucleotide sequence corresponding to the nucleotide sequence substantially as set forth in <400>1 or a nucleotide sequence capable of hybridising to <400>1 under low stringency conditions at 42° C. or a variant, mutant or homologue of said actinomycete.
(b) An actinomycete characterised either by a nucleotide sequence corresponding to the nucleotide sequence substantially as set forth in <400>2 or a nucleotide sequence capable of hybridising to <400>2 under low stringency conditions at 42° C. or a variant, mutant or homologue of said actinomycete.
(c) An actinomycete characterised either by a nucleotide sequence corresponding to the nucleotide sequence substantially as set forth in <400>3 or a nucleotide sequence capable of hybridising to <400>3 under low stringency conditions at 42° C. or a variant, mutant or homologue of said actinomycete.
(d) An actinomycete characterised either by a nucleotide sequence corresponding to the nucleotide sequence substantially as set forth in <400>4 or a nucleotide sequence capable of hybridising to <400>4 under low stringency conditions at 42° C. or a variant, mutant or homologue of said actinomycete.
(e) An actinomycete characterised either by a nucleotide sequence corresponding to the nucleotide sequence substantially as set forth in <400>5 or a nucleotide sequence capable of hybridising to <400>5 under low stringency conditions at 42° C. or a variant, mutant or homologue of said actinomycete.
(f) An actinomycete characterised either by a nucleotide sequence corresponding to the nucleotide sequence substantially as set forth in <400>6 or a nucleotide sequence capable of hybridising to <400>6 under low stringency conditions at 42° C. or a variant, mutant or homologue of said actinomycete.

(g) An actinomycete characterised either by a nucleotide sequence corresponding to the nucleotide sequence substantially as set forth in <400>7 or a nucleotide sequence capable of hybridising to <400>7 under low stringency conditions at 42° C. or a variant, mutant or homologue of said actinomycete.

(h) An actinomycete characterised either by a nucleotide sequence corresponding to the nucleotide sequence substantially as set forth in <400>8 or a nucleotide sequence capable of hybridising to <400>8 under low stringency conditions at 42° C. or a variant, mutant or homologue of said actinomycete
(i) An actinomycete characterised either by a nucleotide sequence corresponding to the nucleotide sequence substantially as set forth in <400>9 or a nucleotide sequence capable of hybridising to <400>9 under low stringency conditions at 42° C. or a variant, mutant or homologue of said actinomycete.
(j) An actinomycete characterised either by a nucleotide sequence corresponding to the nucleotide sequence substantially as set forth in <400>10 or a nucleotide sequence capable of hybridising to <400>10 under low stringency conditions at 42° C. or a variant, mutant or homologue of said actinomycete.
(k) An actinomycete characterised either by a nucleotide sequence corresponding to the nucleotide sequence substantially as set forth in <400>11 or a nucleotide sequence capable of hybridising to <400>11 under low stringency conditions at 42° C. or a variant, mutant or homologue of said actinomycete.
(l) An actinomycete characterised either by a nucleotide sequence corresponding to the nucleotide sequence substantially as set forth in <400>12 or a nucleotide sequence capable of hybridising to <400>12 under low stringency conditions at 42° C. or a variant, mutant or homologue of said actinomycete.
(m) An actinomycete characterised either by a nucleotide sequence corresponding to the nucleotide sequence substantially as set forth in <400>13 or a nucleotide sequence capable of hybridising to <400>13 under low stringency conditions at 42° C. or a variant, mutant or homologue of said actinomycete.
(n) An actinomycete characterised either by a nucleotide sequence corresponding to the nucleotide sequence substantially as set forth in <400>14 or a nucleotide sequence capable of hybridising to <400>14 under low stringency conditions at 42° C. or a variant, mutant or homologue of said actinomycete.
(o) An actinomycete characterised either by a nucleotide sequence corresponding to the nucleotide sequence substantially as set forth in <400>15 or a nucleotide sequence capable of hybridising to <400>15 under low stringency conditions at 42° C. or a variant, mutant or homologue of said actinomycete.
(p) An actinomycete characterised either by a nucleotide sequence corresponding to the nucleotide sequence substantially as set forth in <400>16 or a nucleotide sequence capable of hybridising to <400>16 under low stringency conditions at 42° C. or a variant, mutant or homologue of said actinomycete.
(q) An actinomycete characterised either by a nucleotide sequence corresponding to the nucleotide sequence substantially as set forth in <400>17 or a nucleotide sequence capable of hybridising to <400>17 under low stringency conditions at 42° C. or a variant, mutant or homologue of said actinomycete.
(r) An actinomycete characterised either by a nucleotide sequence corresponding to the nucleotide sequence substantially as set forth in <400>18 or a nucleotide sequence capable of hybridising to <400>18 under low stringency conditions at 42° C. or a variant, mutant or homologue of said actinomycete.
(s) An actinomycete characterised either by a nucleotide sequence corresponding to the nucleotide sequence substantially as set forth in <400>19 or a nucleotide sequence capable of hybridising to <400>19 under low stringency conditions at 42° C. or a variant, mutant or homologue of said actinomycete.
(t) An actinomycete characterised either by a nucleotide sequence corresponding to the nucleotide sequence substantially as set forth in <400>20 or a nucleotide sequence capable of hybridising to <400>20 under low stringency conditions at 42° C. or a variant, mutant or homologue of said actinomycete.
(u) An actinomycete characterised either by a nucleotide sequence corresponding to the nucleotide sequence substantially as set forth in <400>21 or a nucleotide sequence capable of hybridising to <400>21 under low stringency conditions at 42° C. or a variant, mutant or homologue of said actinomycete.
(v) An actinomycete characterised either by a nucleotide sequence corresponding to the nucleotide sequence substantially as set forth in <400>22 or a nucleotide sequence capable of hybridising to <400>22 under low stringency conditions at 42° C. or a variant, mutant or homologue of said actinomycete.
(w) An actinomycete characterised either by a nucleotide sequence corresponding to the nucleotide sequence substantially as set forth in <400>23 or a nucleotide sequence capable of hybridising to <400>23 under low stringency conditions at 42° C. or a variant, mutant or homologue of said actinomycete.
(x) An actinomycete characterised either by a nucleotide sequence corresponding to the nucleotide sequence substantially as set forth in <400>24 or a nucleotide sequence capable of hybridising to <400>24 under low stringency conditions at 42° C. or a variant, mutant or homologue of said actinomycete.
(y) An actinomycete characterised either by a nucleotide sequence corresponding to the nucleotide sequence substantially as set forth in <400>25 or a nucleotide sequence capable of hybridising to <400>25 under low stringency conditions at 42° C. or a variant, mutant or homologue of said actinomycete.
(z) An actinomycete characterised either by a nucleotide sequence corresponding to the nucleotide sequence substantially as set forth in <400>26 or a nucleotide sequence capable of hybridising to <400>26 under low stringency conditions at 42° C. or a variant, mutant or homologue of said actinomycete.
(aa) An actinomycete characterised either by a nucleotide sequence corresponding to the nucleotide sequence substantially as set forth in <400>27 or a nucleotide sequence capable of hybridising to <400>27 under low stringency conditions at 42° C. or a variant, mutant or homologue of said actinomycete.
(ab) An actinomycete characterised either by a nucleotide sequence corresponding to the nucleotide sequence substantially as set forth in <400>28 or a nucleotide sequence capable of hybridising to <400>28 under low stringency conditions at 42° C. or a variant, mutant or homologue of said actinomycete.
(ac) An actinomycete characterised either by a nucleotide sequence corresponding to the nucleotide sequence substantially as set forth in <400>29 or a nucleotide sequence capable of hybridising to <400>29 under low stringency conditions at 42° C. or a variant, mutant or homologue of said actinomycete.
(ad) An actinomycete characterised either by a nucleotide sequence corresponding to the nucleotide sequence substantially as set forth in <400>30 or a nucleotide sequence capable of hybridising to <400>30 under low stringency conditions at 42° C. or a variant, mutant or homologue of said actinomycete.

A further aspect of the present invention provides a method of improving cereal plant productivity said method comprising introducing into said cereal plant or propagation material thereof:
(i) an effective number of actinomycetes selected from EN2, EN3, EN5, EN6, EN7, EN9, EN16, EN17, EN19, EN23, EN26, EN27, EN28, EN35, EN39, EN46, EN57, EN60, SE1, SE2, PM36, PM40, PM41, PM87, PM144, PM171, PM185, PM208, PM228, PM252 AND PM342 or variants, mutants or homologues thereof; and/or

(ii) an effective amount of one or more metabolites derived from the actinomycetes of (i) or derivative, homologue, analogue, chemical equivalent or mimetic thereof; for a time and under conditions sufficient to induce in the subject cereal plant bio-control activity.

Another further aspect of the present invention provides a method of improving cereal plant productivity said method comprising introducing into said cereal plant or propagation material thereof:
(i) an effective number of actinomycetes selected from EN2, EN3, EN6, EN9, EN16, EN27, EN57, EN60, SE1, SE2, PM87, PM185 and PM208 or variants, mutants or homologues thereof; and/or
(ii) an effective amount of one or more metabolites derived from the actinomycetes of (i) or derivative, homologue, analogue, chemical equivalent or mimetic thereof;
   for a time and under conditions sufficient to induce in the subject cereal plant growth promotion.

Still another further aspect of the present invention provides a method for improving cereal plant productivity said method comprising introducing into said cereal plant or propagation material thereof:
(i) an effective number of actinomycetes selected from EN2, EN3, EN9, EN16, EN23, EN27, EN28, EN35, EN46, EN57, EN60, SE1, SE2 and PM87 or variants, mutants or homologues thereof, and/or
(ii) an effective amount of one or more metabolites derived from the actinomycetes of (i) or derivative, homologue, analogue, chemical equivalent or mimetic thereof;
   for a time and under conditions sufficient to induce in a subject plant both growth promoting activity and bio-control activity.

In yet still another further aspect there is provided a method of improving cereal plant productivity said method comprising introducing into said cereal plant productivity said method comprising introducing into said cereal plant or propagation material thereof:

(i) an effective number of actinomycetes selected from EN2, EN3, EN5, EN16, EN17, EN19, EN23, EN27, EN28, EN35, EN46, EN57, PM36, PM40, PM41, PM87, PM110, PM119, PM144, PM171, PM185, PM208, PM228, PM252, PM342, SE1 and SE2 or variants, mutants or homologues thereof; and/or
(ii) an effective amount of one or more metabolites derived from the actinomycetes of (i) or derivative, homologue, analogue, chemical equivalent or mimetic thereof;
   for a time and under conditions sufficient to induce in the subject cereal plant bio-control activity.

Another aspect of the present invention is directed to a method of improving plant productivity said method comprising introducing into said plant or propagation materials thereof:
(i) an effective number of novel endophytic actinomycetes or variants, mutants or homologues thereof; and/or
(ii) an effective amount of one or more metabolites derived from the actinomycetes of (i) or derivative, homologue, analogue, chemical equivalent or mimetic thereof,
   for a time and under conditions sufficient to induce in the subject plant at least one characteristic of improved productivity.

Preferably, said novel endophytic actinomycete is selected from the list consisting of:
(a) An actinomycete characterised either by a nucleotide sequence corresponding to the nucleotide sequence substantially as set forth in <400>1 or a nucleotide sequence capable of hybridising to <400>1 under low stringency conditions at 42° C. or a variant, mutant or homologue thereof.
(b) An actinomycete characterised either by nucleotide sequence corresponding to the nucleotide sequence substantially as set forth in <400>2 or a nucleotide sequence capable of hybridising to <400>2 under low stringency conditions at 42° C. or a variant, mutant or homologue thereof.
(c) An actinomycete characterised either by nucleotide sequence corresponding to the nucleotide sequence substantially as set forth in <400>7 or a nucleotide sequence capable of hybridising to <400>7 under low stringency conditions at 42° C. or a variant, mutant or homologue thereof.
(d) An actinomycete characterised either by nucleotide sequence corresponding to the nucleotide sequence substantially as set forth in <400>10 or a nucleotide sequence capable of hybridising to <400>10 under low stringency conditions at 42° C. or a variant, mutant or homologue thereof.
(e) An actinomycete characterised either by nucleotide sequence corresponding to the nucleotide sequence substantially as set forth in <400>12 or a nucleotide sequence capable of hybridising to <400>12 under low stringency conditions at 42° C. or a variant, mutant or homologue thereof.
(f) An actinomycete characterised either by nucleotide sequence corresponding to the nucleotide sequence substantially as set forth in <400>13 or a nucleotide sequence capable of hybridising to <400>13 under low stringency conditions at 42° C. or a variant, mutant or homologue thereof.
(g) An actinomycete characterised either by nucleotide sequence corresponding to the nucleotide sequence substantially as set forth in <400>16 or a nucleotide sequence capable of hybridising to <400>16 under low stringency conditions at 42° C. or a variant, mutant or homologue thereof.
(h) An actinomycete characterised either by nucleotide sequence corresponding to the nucleotide sequence substantially as set forth in <400>18 or a nucleotide sequence capable of hybridising to <400>18 under low stringency conditions at 42° C. or a variant, mutant or homologue thereof.
(i) An actinomycete characterised either by nucleotide sequence corresponding to the nucleotide sequence substantially as set forth in <400>24 or a nucleotide sequence capable of hybridising to <400>24 under low stringency conditions at 42° C. or a variant, mutant or homologue thereof.

In yet another most preferred embodiment said actinomycete corresponds to EN2, EN3, EN16, EN23, EN27, EN28, EN46, EN60 or PM87.

In another preferred embodiment, said novel endophytic actinomycete is selected from the list consisting of:
(a) An actinomycete characterised either by nucleotide sequence corresponding to the nucleotide sequence substantially as set forth in <400>3 or a nucleotide sequence capable of hybridising to <400>3 under low stringency conditions at 42° C. or a variant, mutant or homologue thereof.
(b) An actinomycete characterised either by nucleotide sequence corresponding to the nucleotide sequence substantially as set forth in <400>4 or a nucleotide sequence capable of hybridising to <400>4 under low stringency conditions at 42° C. or a variant, mutant or homologue thereof.
(c) An actinomycete characterised either by nucleotide sequence corresponding to the nucleotide sequence substantially as set forth in <400>5 or a nucleotide sequence capable of hybridising to <400>5 under low stringency conditions at 42° C. or a variant, mutant or homologue thereof.
(d) An actinomycete characterised either by nucleotide sequence corresponding to the nucleotide sequence substantially as set forth in <400>6 or a nucleotide sequence capable of hybridising to <400>6 under low stringency conditions at 42° C. or a variant, mutant or homologue thereof.
(e) An actinomycete characterised either by nucleotide sequence corresponding to the nucleotide sequence substantially as set forth in <400>8 or a nucleotide sequence capable of hybridising to <400>8 under low stringency conditions at 42° C. or a variant, mutant or homologue thereof.

(f) An actinomycete characterised either by nucleotide sequence corresponding to the nucleotide sequence substantially as set forth in <400>9 or a nucleotide sequence capable of hybridising to <400>9 under low stringency conditions at 42° C. or a variant, mutant or homologue thereof.

(g) An actinomycete characterised either by nucleotide sequence corresponding to the nucleotide sequence substantially as set forth in <400>11 or a nucleotide sequence capable of hybridising to <400>11 under low stringency conditions at 42° C. or a variant, mutant or homologue thereof.

(h) An actinomycete characterised either by nucleotide sequence corresponding to the nucleotide sequence substantially as set forth in <400>14 or a nucleotide sequence capable of hybridising to <400>14 under low stringency conditions at 42° C. or a variant, mutant or homologue thereof.
(i) An actinomycete characterised either by nucleotide sequence corresponding to the nucleotide sequence substantially as set forth in <400>15 or a nucleotide sequence capable of hybridising to <400>15 under low stringency conditions at 42° C. or a variant, mutant or homologue thereof.
(j) An actinomycete characterised either by nucleotide sequence corresponding to the nucleotide sequence substantially as set forth in <400>17 or a nucleotide sequence capable of hybridising to <400>17 under low stringency conditions at 42° C. or a variant, mutant or homologue thereof.
(k) An actinomycete characterised either by nucleotide sequence corresponding to the nucleotide sequence substantially as set forth in <400>19 or a nucleotide sequence capable of hybridising to <400>19 under low stringency conditions at 42° C. or a variant, mutant or homologue thereof.

(l) An actinomycete characterised either by nucleotide sequence corresponding to the nucleotide sequence substantially as set forth in <400>20 or a nucleotide sequence capable of hybridising to <400>20 under low stringency conditions at 42° C. or a variant, mutant or homologue thereof.
(m) An actinomycete characterised either by nucleotide sequence corresponding to the nucleotide sequence substantially as set forth in <400>21 or a nucleotide sequence capable of hybridising to <400>21 under low stringency conditions at 42° C. or a variant, mutant or homologue thereof.
(n) An actinomycete characterised either by nucleotide sequence corresponding to the nucleotide sequence substantially as set forth in <400>22 or a nucleotide sequence capable of hybridising to <400>22 under low stringency conditions at 42° C. or a variant, mutant or homologue thereof.
(o) An actinomycete characterised either by nucleotide sequence corresponding to the nucleotide sequence substantially as set forth in <400>23 or a nucleotide sequence capable of hybridising to <400>23 under low stringency conditions at 42° C. or a variant, mutant or homologue thereof.
(p) An actinomycete characterised either by nucleotide sequence corresponding to the nucleotide sequence substantially as set forth in <400>25 or a nucleotide sequence capable of hybridising to <400>25 under low stringency conditions at 42° C. or a variant, mutant or homologue thereof.
(q) An actinomycete characterised either by nucleotide sequence corresponding to the nucleotide sequence substantially as set forth in <400>25 or a nucleotide sequence capable of hybridising to <400>25 under low stringency conditions at 42° C. or a variant, mutant or homologue thereof.
(r) An actinomycete characterised either by nucleotide sequence corresponding to the nucleotide sequence substantially as set forth in <400>26 or a nucleotide sequence capable of hybridising to <400>26 under low stringency conditions at 42° C. or a variant, mutant or homologue thereof.
(s) An actinomycete characterised either by nucleotide sequence corresponding to the nucleotide sequence substantially as set forth in <400>27 or a nucleotide sequence capable of hybridising to <400>27 under low stringency conditions at 42° C. or a variant, mutant or homologue thereof.
(t) An actinomycete characterised either by nucleotide sequence corresponding to the nucleotide sequence substantially as set forth in <400>28 or a nucleotide sequence capable of hybridising to <400>28 under low stringency conditions at 42° C. or a variant, mutant or homologue thereof.
(u) An actinomycete characterised either by nucleotide sequence corresponding to the nucleotide sequence substantially as set forth in <400>29 or a nucleotide sequence capable of hybridising to <400>29 under low stringency conditions at 42° C. or a variant, mutant or homologue thereof.

In yet another aspect the present invention is directed to the cereal plant-derived endophytic actinomycetes or variants, mutants or homologues thereof or metabolites derived therefrom or derivatives, homologues, analogues, chemical equivalents or mimetics thereof for use in the method of the present invention.

In yet still another aspect there is provided an agricultural composition comprising the endophytic actinomycetes hereinbefore described or metabolites derived therefrom together with one or more agriculturally acceptable carriers and/or diluents.

Another aspect of the present invention is directed to a novel, isolated plant-derived endophytic actinomycete or variant, mutant or homologue thereof.

More particularly, the present invention is directed to a novel, isolated cereal plant-derived endophytic actinomycete or variant, mutant or homologue thereof.

The present invention still more particularly provides a novel, isolated wheat plant-derived endophytic actinomycete or variant, mutant or homologue thereof.

In one aspect, the present invention provides an isolated actinomycete wherein said actinomycete is characterised either by a nucleotide sequence corresponding to the nucleotide sequence substantially as set forth in <400>1 or a nucleotide sequence capable of hybridising to <400>1 under low stringency conditions at 42° C. or a variant, mutant or homologue of said actinomycete.

Preferably, the subject actinomycete corresponds to EN2 (AGAL Deposit No. NM03/35895).

In another aspect the present invention provides an isolated actinomycete wherein said actinomycete is characterised either by a nucleotide sequence corresponding to the nucleotide sequence substantially as set forth in <400>2 or a nucleotide sequence capable of hybridising to <400>2 under low stringency conditions at 42° C. or a variant, mutant or homologue of said actinomycete.

Preferably, the subject actinomycete corresponds to EN3 (AGAL Deposit No. NM03/36501).

In yet another aspect the present invention provides an isolated actinomycete wherein said actinomycete is characterised either by a nucleotide sequence corresponding to the nucleotide sequence substantially as set forth in <400>7 or a nucleotide sequence capable of hybridising to <400>7 under low stringency conditions at 42° C. or a variant, mutant or homologue of said actinomycete.

Preferably, the subject actinomycete corresponds to EN16 (AGAL Deposit No. NM03/35604).

In still another aspect the present invention provides an isolated actinomycete wherein said actinomycete is characterised either by a nucleotide sequence corresponding to the nucleotide sequence substantially as set forth in <400>10 or a nucleotide sequence capable of hybridising to <400>10 under low stringency conditions at 42° C. or a variant, mutant or homologue of said actinomycete.

Preferably, the subject actinomycete corresponds to EN23 (AGAL Deposit No. NM03/35605).

In yet still another aspect the present invention provides an isolated actinomycete wherein said actinomycete is characterised either by a nucleotide sequence corresponding to the nucleotide sequence substantially as set forth in <400>12 or a nucleotide sequence capable of hybridising to <400>12 under low stringency conditions at 42° C. or a variant, mutant or homologue of said actinomycete.

Preferably, the subject actinomycete corresponds to EN27 (AGAL Deposit No. NM03/35606).

In still yet another aspect the present invention provides an isolated actinomycete wherein said actinomycete is characterised either by a nucleotide sequence corresponding to the nucleotide sequence substantially as set forth in <400>13 or a nucleotide sequence capable of hybridising to <400>13 under low stringency conditions at 42° C. or a variant, mutant or homologue of said actinomycete.

Preferably, the subject actinomycete corresponds to EN28 (AGAL Deposit No. NM03/35607).

In yet another further aspect the present invention provides an isolated actinomycete wherein said actinomycete is characterised either by a nucleotide sequence corresponding to the nucleotide sequence substantially as set forth in <400>16 or a nucleotide sequence capable of hybridising to <400>16 under low stringency conditions at 42° C. or a variant, mutant or homologue of said actinomycete.

Preferably, the subject actinomycete corresponds to EN46 (AGAL Deposit No. NM03/35609).

In still another further aspect the present invention provides an isolated actinomycete wherein said actinomycete is characterised either by a nucleotide sequence corresponding to the nucleotide sequence substantially as set forth in <400>18 or a nucleotide sequence capable of hybridising to <400>18 under low stringency conditions at 42° C. or a variant, mutant or homologue of said actinomycete.

Preferably, the subject actinomycete corresponds to EN60 (AGAL Deposit No. NM03/35896).

In yet still another aspect the present invention provides an isolated actinomycete wherein said actinomycete is characterised either by a nucleotide sequence corresponding to the nucleotide sequence substantially as set forth in <400>24 or a nucleotide sequence capable of hybridising to <400>24 under low stringency conditions at 42° C. or a variant, mutant or homologue of said actinomycete.

Preferably, the subject actinomycete corresponds to PM87 (AGAL Deposit No. NM03/35608).

In another aspect the present invention provides an isolated actinomycete wherein said actinomycete is characterised either by a nucleotide sequence corresponding to the nucleotide sequence substantially as set forth in <400>3 or a nucleotide sequence capable of hybridising to <400>3 under low stringency conditions at 42° C. or a variant, mutant or homologue of said actinomycete.

Preferably, the subject actinomycete corresponds to EN5.

In yet another aspect the present invention provides an isolated actinomycete wherein said actinomycete is characterised either by a nucleotide sequence corresponding to the nucleotide sequence substantially as set forth in <400>4 or a nucleotide sequence capable of hybridising to <400>4 under low stringency conditions at 42° C. or a variant, mutant or homologue of said actinomycete.

Preferably, the subject actinomycete corresponds to EN6.

In still another aspect the present invention provides an isolated actinomycete wherein said actinomycete is characterised either by a nucleotide sequence corresponding to the nucleotide sequence substantially as set forth in <400>5 or a nucleotide sequence capable of hybridising to <400>5 under low stringency conditions at 42° C. or a variant, mutant or homologue of said actinomycete.

Preferably, the subject actinomycete corresponds to EN7.

In yet still another aspect the present invention provides an isolated actinomycete wherein said actinomycete is characterised either by a nucleotide sequence corresponding to the nucleotide sequence substantially as set forth in <400>6 or a nucleotide sequence capable of hybridising to <400>6 under low stringency conditions at 42° C. or a variant, mutant or homologue of said actinomycete.

Preferably, the subject actinomycete corresponds to EN9.

In a further aspect the present invention provides an isolated actinomycete wherein said actinomycete is characterised either by a nucleotide sequence corresponding to the nucleotide sequence substantially as set forth in <400>8 or a nucleotide sequence capable of hybridising to <400>8 under low stringency conditions at 42° C. or a variant, mutant or homologue of said actinomycete.

Preferably, the subject actinomycete corresponds to EN17.

In another further aspect the present invention provides an isolated actinomycete wherein said actinomycete is characterised either by a nucleotide sequence corresponding to the nucleotide sequence substantially as set forth in <400>9 or a nucleotide sequence capable of hybridising to <400>9 under low stringency conditions at 42° C. or a variant, mutant or homologue of said actinomycete.

Preferably, the subject actinomycete corresponds to EN19.

In yet another aspect the present invention provides an isolated actinomycete wherein said actinomycete is characterised either by a nucleotide sequence corresponding to the nucleotide sequence substantially as set forth in <400>11 or a nucleotide sequence capable of hybridising to <400>11 under low stringency conditions at 42° C. or a variant, mutant or homologue of said actinomycete.

Preferably, the subject actinomycete corresponds to EN26.

In still another aspect the present invention provides an isolated actinomycete wherein said actinomycete is characterised either by a nucleotide sequence corresponding to the nucleotide sequence substantially as set forth in <400>14 or a nucleotide sequence capable of hybridising to <400>14 under low stringency conditions at 42° C. or a variant, mutant or homologue of said actinomycete.

Preferably, the subject actinomycete corresponds to EN35.

In yet still another aspect the present invention provides an isolated actinomycete wherein said actinomycete is characterised either by a nucleotide sequence corresponding to the nucleotide sequence substantially as set forth in <400>15 or a nucleotide sequence capable of hybridising to <400>15 under low stringency conditions at 42° C. or a variant, mutant or homologue of said actinomycete.

Preferably, the subject actinomycete corresponds to EN39.

In still yet another aspect the present invention provides an isolated actinomycete wherein said actinomycete is characterised either by a nucleotide sequence corresponding to the nucleotide sequence substantially as set forth in <400>17 or a nucleotide sequence capable of hybridising to <400>17 under low stringency conditions at 42° C. or a variant, mutant or homologue of said actinomycete.

Preferably, the subject actinomycete corresponds to EN57.

In another aspect the present invention provides an isolated actinomycete wherein said actinomycete is characterised either by a nucleotide sequence corresponding to the nucleotide sequence substantially as set forth in <400>19 or a nucleotide sequence capable of hybridising to <400>19 under low stringency conditions at 42° C. or a variant, mutant or homologue of said actinomycete.

Preferably, the subject actinomycete corresponds to SE1.

In yet another aspect the present invention provides an isolated actinomycete wherein said actinomycete is characterised either by a nucleotide sequence corresponding to the nucleotide sequence substantially as set forth in <400>20 or a nucleotide sequence capable of hybridising to <400>20 under low stringency conditions at 42° C. or a variant, mutant or homologue of said actinomycete.

Preferably, the subject actinomycete corresponds to SE2.

In still another aspect the present invention provides an isolated actinomycete wherein said actinomycete is characterised either by a nucleotide sequence corresponding to the nucleotide sequence substantially as set forth in <400>21 or a nucleotide sequence capable of hybridising to <400>21 under low stringency conditions at 42° C. or a variant, mutant or homologue of said actinomycete.

Preferably, the subject actinomycete corresponds to PM36.

In yet still another aspect the present invention provides an isolated actinomycete wherein said actinomycete is characterised either by a nucleotide sequence corresponding to the nucleotide sequence substantially as set forth in <400>22 or a nucleotide sequence capable of hybridising to <400>22 under low stringency conditions at 42° C. or a variant, mutant or homologue of said actinomycete.

Preferably, the subject actinomycete corresponds to PM40.

In a further aspect the present invention provides an isolated actinomycete wherein said actinomycete is characterised either by a nucleotide sequence corresponding to the nucleotide sequence substantially as set forth in <400>23 or a nucleotide sequence capable of hybridising to <400>23 under low stringency conditions at 42° C. or a variant, mutant or homologue of said actinomycete.

Preferably, the subject actinomycete corresponds to PM41.

In another further aspect the present invention provides an isolated actinomycete wherein said actinomycete is characterised either by a nucleotide sequence corresponding to the nucleotide sequence substantially as set forth in <400>24 or a nucleotide sequence capable of hybridising to <400>24 under low stringency conditions at 42° C. or a variant, mutant or homologue of said actinomycete.

Preferably, the subject actinomycete corresponds to PM87.

In still another aspect the present invention provides an isolated actinomycete wherein said actinomycete is characterised either by a nucleotide sequence corresponding to the nucleotide sequence substantially as set forth in <400>25 or a nucleotide sequence capable of hybridising to <400>25 under low stringency conditions at 42° C. or a variant, mutant or homologue of said actinomycete.

Preferably, the subject actinomycete corresponds to PM171.

In yet still another aspect the present invention provides an isolated actinomycete wherein said actinomycete is characterised either by a nucleotide sequence corresponding to the nucleotide sequence substantially as set forth in <400>26 or a nucleotide sequence capable of hybridising to <400>26 under low stringency conditions at 42° C. or a variant, mutant or homologue of said actinomycete.

Preferably, the subject actinomycete corresponds to PM185.

In still yet another aspect the present invention provides an isolated actinomycete wherein said actinomycete is characterised either by a nucleotide sequence corresponding to the nucleotide sequence substantially as set forth in <400>27 or a nucleotide sequence capable of hybridising to <400>27 under low stringency conditions at 42° C. or a variant, mutant or homologue of said actinomycete.

Preferably, the subject actinomycete corresponds to PM208.

In another aspect the present invention provides an isolated actinomycete wherein said actinomycete is characterised either by a nucleotide sequence corresponding to the nucleotide sequence substantially as set forth in <400>28 or a nucleotide sequence capable of hybridising to <400>28 under low stringency conditions at 42° C. or a variant, mutant or homologue of said actinomycete.

Preferably, the subject actinomycete corresponds to PM228.

In yet another aspect the present invention provides an isolated actinomycete wherein said actinomycete is characterised either by a nucleotide sequence corresponding to the nucleotide sequence substantially as set forth in <400>29 or a nucleotide sequence capable of hybridising to <400>29 under low stringency conditions at 42° C. or a variant, mutant or homologue of said actinomycete.

Preferably, the subject actinomycete corresponds to PM252.

In still another aspect the present invention provides an isolated actinomycete wherein said actinomycete is characterised either by a nucleotide sequence corresponding to the nucleotide sequence substantially as set forth in <400>30 or a nucleotide sequence capable of hybridising to <400>30 under low stringency conditions at 42° C. or a variant, mutant or homologue of said actinomycete.

Preferably, the subject actinomycete corresponds to PM342.

Yet another aspect of the present invention is directed to metabolites derived from the novel actinomycetes hereinbefore defined and derivatives, homologues, analogues, chemical equivalents, mutants and mimetics of said metabolites.

Yet another aspect of the present invention is directed to antibodies to the novel actinomycetes or metabolites hereinbefore defined or derivative, homologue, analogue, chemical equivalent, or mimetic of said antibody. Accordingly, still another aspect of the present invention is directed to the use of the novel actinomycetes hereinbefore defined and metabolites derived therefrom in relation to therapeutic and prophylactic applications in respect of both medical purposes and for nay non-medical purpose such as agricultural purposes.

### BRIEF DESCRIPTION OF THE DRAWINGS

FIG. 1 is a graphical representation of the disease control levels of those isolates with statistically significant results in the field soil trial.

FIG. 2 is a schematic representation of the primers used for complete 16S DNA sequencing.

FIG. 3 is a graphical representation of the grain yield compared to untreated control at Alford Site 2002 with Take-all disease. Red bars indicate statistically significant yield increases after analysis with ANOVA.

FIG. 4 is a graphical representation of the effect of actinomycete seed inoculation on height of wheat grown in soil infested with Pythium irregulare. Red bars indicate statistically significant yield increases (P<0.01) after analysis with ANOVA FIG. 5 is a graphical representation of the effect of actinomycete seed inoculation on germination and emergence of wheat grown in soil infested with Pythium irregulare. Red bars indicate statistically significant yield increases (P<0.01) after analysis with ANOVA.

FIG. 6 is a graphical representation of the growth of wheat seeds inoculated with actinobacterial spores and an uninoculated control (+Py) in soil infested with Pythium irregulare. A control treatment with no disease or actinobacteria inoculation was also included (−Py).

FIG. 7 is a graphical representation of the growth of wheat seeds inoculated with actinobacterial spores and an uninoculated control (+Py) in soil infested with Pythium irregulare. A control treatment with no disease or actinobacteria inoculation was also included (−Py). The plants were grown at 21° C. instead of 12° C. Growth was measured as dry weight of the root or shoot.

FIG. 8 is a schematic representation of neighbour-joining phylogenetic tree of the full 16S rDNA sequences from selected isolates. The sequence data for several closely related actinobacterial type cultures were recovered from GenBank and included in the tree. The accession numbers for the sequences are: Bacillus subtilis NC_{—}000964 (Region: 9809 . . . 11361), Microbispora amethystogenes U48988, Nocardioides albus X53211, S. scabiesD63862; S. galilaeus AB045878; S. argenteolus AB045872; S. setonii D63872; S. caviscabies AF112160,1 Streptosporangiacae str. PA147 AF223347. The bootstrap values from 5000 pseudo-replications are shown at each of the branch points on the tree.

FIG. 9 is a graphical representation of the relative density of aphids on plants treated with endophytes.

FIG. 10 is an image of egfp-expressing Streptomyces sp. EN27 under the LSCM at 1800× magnification.

FIG. 11 is an image of egp-expressing Streptomyces sp. EN27 in a 24 h old wheat embryo. FIG. 11.1 shows the image under blue excitation/green emission, FIG. 6.2 shows the image under UV excitation/blue emission. FIG. 11.3 shows an image enhanced merge or the two images, 11.1 shown in green, while 11.2 is shown in red. All images are at 400× magnification. EM-embryonic wheat tissue, EN27-egfp expressing endophytic actinomycete (colour image available upon request).

FIG. 12 is an image of egfp-expressing Streptomyces sp. EN27 microcolonies in 3 day old wheat embryo tissue (plumule). FIG. 12.1 shows the image under blue excitation/green emission, FIG. 12.2 shows the image under UV excitation/blue emission. FIG. 12.3 shows an image enhanced merge of the two images, 12.1 shown in green, while 12.2 is shown in red. All images are at 400× magnification. EM-embryonic wheat tissue, EN27-egfp-expressing endophytic actinomycete (colour image available upon request).

FIG. 13 is an image of egfp-expressing Streptomyces sp. EN27 microcolony in the emerging radicle. FIG. 13.1 shows the image under blue excitation/green emission, FIG. 13.2 shows the image under UV excitation/blue emission. FIG. 13.3 shows an image enhanced merge of the two images, 13.1 shown in green, while 13.2 is shown in red. All images are at 200× magnification. RA-radicle, EN27-egfp-expressing endophytic actinomycete (colour image available upon request).

FIG. 14 is an image of egfp-expressing Streptomyces sp. EN27 microcolonies in the endosperm after 3 days. FIG. 14.1 shows the image under blue excitation/green emission,

FIG. 14.2 shows the image under UV excitation/blue emission. FIG. 14.3 shows an image enhanced merge of the two images, 14.1 shown in green, while 14.2 is shown in red. All images are at 200× magnification. AL-aleurone, ES-endosperm, PE-pericarp, EN27-egfp-expressing endophytic actinomycete (colour image available upon request).

FIG. 15 is a schematic representation of the sequences of actinomycete isolates EN2, EN3, EN16, EN23, EN27, EN28, EN46, EN60, PM87.

FIG. 16 is a schematic representation of the sequences of actinomycete isolates EN5, EN6, EN7, EN9, EN17, EN19, EN26, EN35, EN39, EN57.

FIG. 17 is a schematic representation of the sequences of actinomycete isolates SE1 and SE2.

FIG. 18 is a schematic representation of the sequences of actinomycete isolates PM36, PM40, PM41, PM144, PM171, PM185, PM208, PM228, PM252, PM342.

FIG. 19 is a schematic representation of the sequences of actinomycete isolates EN4, EN10, EN22, EN30, EN43, EN47 and EN59.

It should be understood that in each of FIGS. 15-18, and the sequence listing attached herewith, “n” is an unknown nucleotide and, in accordance with IUB notation, “m” is adenine or cytosine, “k” is guanine or thymine and “w” is adenine or thymine.

FIG. 20 is a graphical representation of indole acetic acid production.

FIG. 21 is a graphical representation showing T-RFLP HnfI profiles for the roots of wheat grown from (a) uninoculated seed, (b) Microbispora sp. EN2 inoculated seed, (c) Streptomyces sp. EN27 inoculated seed and (c) Nocardioides albus EN46 inoculated seed. The highlighted peaks correspond to the specific fragment of the actinobacterial endophyte inoculated onto the seed.

### DETAILED DESCRIPTION OF THE INVENTION

The present invention is predicated, in part, on the surprising and unexpected determination:
(i) that a proportion of wheat plants are, in fact, colonised by actinomycete species which are either novel or else were not previously known to exhibit the capacity to exist in an endophytic relationship; and
(ii) that only some of these wheat plant endophytic actinomycetes also function as modulators of improved plant productivity and provide growth promotion advantages (such as improved seed germination) and/or bio-control advantages to that plant, which advantages are not seen in wheat plants lacking the subject endophytic actinomycetes.

This has now led to the development of methodology which facilitates the routine cultivation of plants, in particular cereal crops, which exhibit growth productivity advantages due to introduction into the plant a population of actinomycetes and/or their metabolites which have been identified by the inventors, in accordance with the present invention, to both form an endophytic relationship with the plant and provide the above-identified productivity advantages.

Accordingly, one aspect of the present invention is directed to a method of improving plant productivity said method comprising introducing into said plant or propagation material thereof:
(i) an effective number of cereal plant-derived endophytic actinomycetes or variants, mutants or homologues thereof; which actinomycetes facilitate induction of at least one characteristic related to improved productivity; and/or
(ii) an effective amount of one or more metabolites derived from the actinomycetes of (i) or derivative, homologue, analogue, chemical equivalent or mimetic thereof; for a time and under conditions sufficient to induce, in the subject plant, said characteristic.

Reference to a “plant” should be understood as a reference to any naturally or non-naturally occurring plant in respect of which improved productivity is sought. For example, flowering crops, cereal crops (eg. wheat, barley, rye, triticale maize, oats, canary seed, sorghum, millet and rice) and horticultural crops (eg. tomatoes, onion, potato, peanut, chickpea, pea, lentil, mung bean, faba bean, canola, linola, mustard, sunflower, safflower, soybean, lupins and cotton). By “non-naturally” is meant that the subject plant has undergone some form of manipulation of modification prior to treatment in accordance with the method of the present invention. Examples of manipulation include, but are not limited to, genetic modification of a plant or treatment of a seedling or propagating material with an extraneous proteinaceous or non-proteinaceous molecule such as Bacillus thuringiensis toxin, genes for provitamin A synthesis, genes for vitamin E synthesis, protease inhibitors or genes for virus coat proteins. Although some manipulations, such as genetic modification, may lead to the improvement of a productivity characteristic, such modification may be of limited value due to its improvement of only some of the desired productivity characteristics. For example, where a genetic modification is introduced to provide a plant with certain bio-control characteristics, the subject plant may still not exhibit other desirable productivity characteristics such as improved plant vigour or yield. In this case, these latter productivity improvements can be achieved by treating the genetically modified plant in accordance with the method of the present invention to induce early plant vigour or increased yield, for example. The non-naturally occurring plant may be derived from any source. For example, to the extent that the non-naturally occurring plant is one which is genetically modified, the plant may be one which has itself undergone genetic modification or it may have been cultivated from a seed which has undergone genetic modification. Alternatively, the plant may be derived from a seed which itself was itself derived from a genetically modified plant. Preferably, the plant is a cereal crop and even more particularly a wheat crop, barley crop, maize, triticale, rye, oats, canary, sorghum, millet or rice.

Reference to “propagation material” should be understood as a reference to any type of cellular material from which a plant would germinate or otherwise arise. Examples of propagating material include, but are not limited to, a seed, cutting, cell suspension, callus culture, tissue culture, protocorm, explants or germplasm. The propagating material may take any suitable form. For example, it may have been freshly harvested or it may be derived from a stock sample, such as a seed sample or a frozen stock of cells.

Accordingly, the present invention more particularly provides a method of improving cereal plant productivity said method comprising introducing into said cereal plant or propagation material thereof:
(i) an effective number of cereal plant-derived endophytic actinomycetes or variants, mutants or homologues thereof; which actinomycetes facilitate induction of at least one characteristic related to improved productivity; and/or
(ii) an effective amount of one or more metabolites derived from the actinomycetes of (i) or derivative, homologue, analogue, chemical equivalent or mimetic thereof;
   for a time and under conditions sufficient to induce, in the subject cereal plant, said characteristic.

Preferably, said cereal plant is a wheat plant, barley, maize, triticale, rye, oats, canary, sorghum, millet or rice plant.

Reference to “metabolite” should be understood as a reference to any proteinaceous or non-proteinaceous molecule produced by the subject endophytic actinomycetes or produced by the plant in response to the actinomycete colonisation or actinomycete metabolite actions which directly or indirectly modulate the metabolism or other functional activity of the host plant. It should be understood, for example, that a molecule which functions as a bio-control agent is an example of a metabolite which is functioning indirectly since it acts to down-regulate or otherwise inhibit the detrimental actions of a pathogen, which pathogenic activity would otherwise adversely affect the viability/health of the host plant. An example of a metabolite which functions directly is a molecule which acts directly on a host plant cell to upregulate its proliferation and/or differentiation, for example thereby providing a growth promoting activity such as promotion of germination. Examples of such metabolites includes, but is not limited to, auxins, gibberellins, cytokinins, indole acetic acid and kinetin.

Reference to “improving plant productivity” should be understood as a reference to achieving a level of productivity in treated plants which is greater than the level of productivity which would be observed in untreated plants. By “productivity” is meant any aspect of the subject plant's development. For example, reference to productivity includes, but is not limited to, growth promotion characteristics such as rate of growth, plant vigour, yield of flower/fruit/grain, health or viability of crop (for example due to reduction in the application of fertilisers and/or chemical pesticides, increased nutrient uptake, increased systemic resistance or herbicidal resistance) and improved seed germination or bio-control characteristics such as those which lead to reduction in disease by decreasing susceptibility to infection by pathogens and/or increasing clearance of existing infections. Reference to “improving” productivity should be understood to include either:
(i) inducing a given productivity characteristic to occur at a level or degree which is greater than that which would be observed in a corresponding, healthy plant which is not treated according to the method of the present invention. An example of this occurring would be the induction of increased yield due to the synthesis of metabolites by the endophytic actinomycetes which facilitate yield production at a level greater than that which is normally observed or the induction of bio-control characteristics which are not normally exhibited by the subject plant; and/or
(ii) facilitating the induction of a given characteristic at a level or degree which one would normally expect to occur in a corresponding healthy, untreated plant but which characteristic is not observed due to extraneous, unexpected or abnormal events. For example, inducing a normal level of vigour in plants which are cultivated in poor quality soils and could not otherwise achieve a normal level of vigour or providing a plant with bio-control characteristics which enables a normal rate of plant development to thereby occur despite the presence of pathogenic microorganisms which would usually adversely affect the rate of development in a corresponding untreated plant.

Reference to a “characteristic related to improved productivity” should therefore be understood to mean any feature which directly or indirectly contributes to a plant's overall productivity. Preferably, the subject characteristic is growth promotion and/or bio-control activity.

Accordingly, in a preferred embodiment there is provided a method of improving plant productivity said method comprising introducing into said plant or propagation material thereof:
(i) an effective number of cereal plant-derived endophytic actinomycetes or variants, mutants or homologues thereof; which actinomycetes facilitate induction of at least one characteristic related to improved productivity; and/or
(ii) an effective amount of one or more metabolites derived from the actinomycetes of (i) or derivative, homologue, analogue, chemical equivalent or mimetic thereof;
   for a time and under conditions sufficient to induce, in the subject plant, growth promotion and/or bio-control activity.

Preferably said plant is a cereal crop and even more preferably a wheat plant or a barley plant.

By “facilitate” is meant that the subject endophytic actinomycete metabolite or metabolites either directly or indirectly induces occurrence of the subject characteristic. For example, in relation to the induction of bio-control characteristics, the endophytic actinomycete may secrete an expression product which is itself directly toxic to a given pathogen.

Alternatively, the endophytic actinomycete may secrete an expression product which acts on the host plant to signal/induce the subject plant to synthesise an expression product which is toxic or inhibitory to the pathogen of interest. The former scenario is an example of a direct relationship while the latter is an example of an indirect relationship.

The present invention is predicated on the surprising determination that some wheat plants are host to endophytic actinomycete species and that some of these species in fact facilitate the induction of one or more characteristics of improved productivity. Accordingly, by “cereal plant-derived endophytic actinomycete” is meant a species of actinomycete which can be found in a cereal plant (although not necessarily all cereal plants), and in particular in wheat plants, and which actinomycetes exhibit the functional activity of facilitating the induction of at least one characteristic of improved productivity. Reference to “facilitating induction of at least one characteristic related to improved productivity” should be understood to have the same meaning as hereinbefore provided. It should also be understood that the subject actinomycete may be isolated from any suitable source and, in accordance with the present invention, is not necessarily required to be isolated specifically from cereal crops. For example, an actinomycete species of interest may be sourced from any naturally or non-naturally occurring source. It should also be understood that any reference herein to a particular species should also be understood to include reference to a related species.

Preferably, the subject cereal plant-derived endophytic actinomycete is an actinomycete species of the genus Microbispora, Streptomyces, Micromonospora, Streptosporangiacae, Nocardiodes, Tsukamurella or Steptosporangium.

Accordingly, in a more preferred embodiment there is provided a method of improving cereal plant productivity said method comprising introducing into said plant or propagation material thereof:
(i) an effective number of endophytic actinomycetes of the genus Microbispora, Streptomyces, Micromonospora, Streptosporangiacae, Nocardiodes, Tsukamurella or Streptosporangium or variants, mutants or homologues thereof; and/or
(ii) an effective amount of one or more metabolites derived from the actinomycetes of (i) or derivative, homologue, analogue, chemical equivalent or mimetic thereof;
   for a time and under conditions sufficient to induce, in the subject cereal plant, said characteristic.

In a more preferred embodiment, where the endophytic actinomycete is of the genus Streptomyces, said Streptomyces is of the species triticum, caviscabies, setonii, galilaeus, peuceticus, bikiniensis, fimbriatus, pseudovenezuelae, argenteolus, platensis, griseus, lincolnensis or related species.

In another preferred embodiment, where the endophytic actinomycete is of the genus Micromonospora said Micromonospora is of the species peucetica, fulvoviolaceus, yulongensis or related species.

In yet another preferred embodiment, where the endophytic actinomycete is of the genus Nocardiodes said Nocardiodes is of the species fulvus, flavus, luteus, albus or related species.

In still another preferred embodiment, where the endophytic actinomycete is of the genus Microbispora, said Microbispora is of the species amethystogenes or related species.

In yet another preferred embodiment, where the endophytic actinomycete is of the genus Tsukamurella, said Tsukamurella is of the species tyrosinovorans-D1498, IM-7430, pulmonis or related species.

In still another preferred embodiment, where the endophytic actinomycete is of the genus Streptomycetaceae, said Streptomycetaceae is of the species SR11 or related species.

In still yet another preferred embodiment, where the endophytic actinomycete is of the species Streptosporangium, said Streptosporangium is of the genus cinnabarium or related species.

In work leading up to the present invention, the inventors identified cereal plant-derived endophytic actinomycetes which are functional, in terms of growth productivity, in plants. These actinomycete isolates have been deposited and are identified herein by reference to an “EN”, “SE”, “PM” or “SC” numeral.

Without limiting the present invention to any one theory or mode of action, the inventors have characterised the subject actinomycetes based on their 16S rDNA sequences and have determined that EN2, EN3, EN5, EN6, EN7, EN9, EN16, EN17, EN19, EN23, EN26, EN27, EN28, EN35, EN39, EN46, EN57, EN60, SE1, SE2, PM36, PM40, PM41, PM87, PM144, PM171, PM185, PM208, PM228, PM252 AND PM342 correspond to previously unidentified species of actinomycete. The actinomycete isolates described herein are thought to correspond to the actinomycete species as listed below:
(a) Actinomycete isolates EN19, EN23, EN27, EN28, EN35, EN57, EN87, SE1, SE2, PM36, PM40, PM41, PM87, PM110, PM119, PM171, PM228 and PM252 correspond to Streptomyces triticum species.
(b) Actinomycete isolates EN5, EN16, EN17, PM144, PM185, PM208 and PM342 correspond to Streptomyces triticum var. griseoviside.
(c) Actinomycete isolate EN46 corresponds to Nocardioides species and is closely related to Nocardioides albus.
(d) Actinomycete isolates EN3 and EN39 corresponds to Streptomyces galilaeus.
(e) Actinomycete isolate EN60 corresponds to a new species related to Streptomyces argenteolus.
(f) Actinomycete isolate EN2 corresponds to a novel Microbispora species.
(g) Actinomycete isolate EN6 corresponds to a novel species related to Streptomyces pseudovenezuelae.
(h) Actinomycete isolate EN7 is related to Streptomyces lincolnesis.
(i) Actinomycete isolate EN9 is related to Streptomyces bikiniensis.
(j) Actinomycete isolate EN26 is a novel Streptomyces species.

More specifically, and still without limiting the present invention in any way:
(a) EN2 is thought to correspond to a population of actinomycetes comprising the rDNA sequence substantially as set forth in <400>1.
(b) EN3 is thought to correspond to a population of actinomycetes comprising the rDNA sequence substantially as set forth in <400>2.
(c) EN5 is thought to correspond to a population of actinomycetes comprising the rDNA sequence substantially as set forth in <400>3.
(d) EN6 is thought to correspond to a population of actinomycetes comprising the rDNA sequence substantially as set forth in <400>4.
(e) EN7 is thought to correspond to a population of actinomycetes comprising the rDNA sequence substantially as set forth in <400>5.
(f) EN9 is thought to correspond to a population of actinomycetes comprising the rDNA sequence substantially as set forth in <400>6.
(g) EN16 is thought to correspond to a population of actinomycetes comprising the rDNA sequence substantially as set forth in <400>7.
(h) EN17 is thought to correspond to a population of actinomycetes comprising the rDNA sequence substantially as set forth in <400>8.
(i) EN19 is thought to correspond to a population of actinomycetes comprising the rDNA sequence substantially as set forth in <400>9.
(j) EN23 is thought to correspond to a population of actinomycetes comprising the rDNA sequence substantially as set forth in <400>10.
(k) EN26 is thought to correspond to a population of actinomycetes comprising the rDNA sequence substantially as set forth in <400>11.
(l) EN27 is thought to correspond to a population of actinomycetes comprising the rDNA sequence substantially as set forth in <400>12.
(m) EN28 is thought to correspond to a population of actinomycetes comprising the rDNA sequence substantially as set forth in <400>13.
(n) EN35 is thought to correspond to a population of actinomycetes comprising the rDNA sequence substantially as set forth in <400>14.
(o) EN39 is thought to correspond to a population of actinomycetes comprising the rDNA sequence substantially as set forth in <400>15.
(p) EN46 is thought to correspond to a population of actinomycetes comprising the rDNA sequence substantially as set forth in <400>16.
(q) EN57 is thought to correspond to a population of actinomycetes comprising the rDNA sequence substantially as set forth in <400>17.
(r) EN60 is thought to correspond to a population of actinomycetes comprising the rDNA sequence substantially as set forth in <400>18.
(s) SE1 is thought to correspond to a population of actinomycetes comprising the rDNA sequence substantially as set forth in <400>19.
(t) SE2 is thought to correspond to a population of actinomycetes comprising the rDNA sequence substantially as set forth in <400>20.
(u) PM36 is thought to correspond to a population of actinomycetes comprising the rDNA sequence substantially as set forth in <400>21.
(v) PM40 is thought to correspond to a population of actinomycetes comprising the rDNA sequence substantially as set forth in <400>22.
(w) PM41 is thought to correspond to a population of actinomycetes comprising the rDNA sequence substantially as set forth in <400>23.
(x) PM87 is thought to correspond to a population of actinomycetes comprising the rDNA sequence substantially as set forth in <400>24.
(y) PM171 is thought to correspond to a population of actinomycetes comprising the rDNA sequence substantially as set forth in <400>25.
(z) PM185 is thought to correspond to a population of actinomycetes comprising the rDNA sequence substantially as set forth in <400>26.
(aa) PM208 is thought to correspond to a population of actinomycetes comprising the rDNA sequence substantially as set forth in <400>27.
(ab) PM228 is thought to correspond to a population of actinomycetes comprising the rDNA sequence substantially as set forth in <400>28.
(ac) PM252 is thought to correspond to a population of actinomycetes comprising the rDNA sequence substantially as set forth in <400>29.
(ad) PM342 is thought to correspond to a population of actinomycetes comprising the rDNA sequence substantially as set forth in <400>30.

Accordingly, in a preferred embodiment the present invention provides a method of improving plant productivity said method comprising introducing into said plant or propagation material thereof:
(i) an effective number of cereal plant-derived endophytic actinomycetes or variants, mutants or homologues thereof; which actinomycetes facilitate induction of at least one characteristic related to improved productivity; and/or
(ii) an effective amount of one or more metabolites derived from the actinomycetes of (i) or derivative, homologue, analogue, chemical equivalent or mimetic thereof,
   for a time and under conditions sufficient to induce, in the subject cereal plant, said characteristic, wherein said actinomycete is selected from the list of:
(a) An actinomycete characterised either by a nucleotide sequence corresponding to the nucleotide sequence substantially as set forth in <400>1 or a nucleotide sequence capable of hybridising to <400>1 under low stringency conditions at 42° C. or a variant, mutant or homologue of said actinomycete.
(b) An actinomycete characterised either by a nucleotide sequence corresponding to the nucleotide sequence substantially as set forth in <400>2 or a nucleotide sequence capable of hybridising to <400>2 under low stringency conditions at 42° C. or a variant, mutant or homologue of said actinomycete.
(c) An actinomycete characterised either by a nucleotide sequence corresponding to the nucleotide sequence substantially as set forth in <400>3 or a nucleotide sequence capable of hybridising to <400>3 under low stringency conditions at 42° C. or a variant, mutant or homologue of said actinomycete.
(d) An actinomycete characterised either by a nucleotide sequence corresponding to the nucleotide sequence substantially as set forth in <400>4 or a nucleotide sequence capable of hybridising to <400>4 under low stringency conditions at 42° C. or a variant, mutant or homologue of said actinomycete.
(e) An actinomycete characterised either by a nucleotide sequence corresponding to the nucleotide sequence substantially as set forth in <400>5 or a nucleotide sequence capable of hybridising to <400>5 under low stringency conditions at 42° C. or a variant, mutant or homologue of said actinomycete.
(f) An actinomycete characterised either by a nucleotide sequence corresponding to the nucleotide sequence substantially as set forth in <400>6 or a nucleotide sequence capable of hybridising to <400>6 under low stringency conditions at 42° C. or a variant, mutant or homologue of said actinomycete.
(g) An actinomycete characterised either by a nucleotide sequence corresponding to the nucleotide sequence substantially as set forth in <400>7 or a nucleotide sequence capable of hybridising to <400>7 under low stringency conditions at 42° C. or a variant, mutant or homologue of said actinomycete.
(h) An actinomycete characterised either by a nucleotide sequence corresponding to the nucleotide sequence substantially as set forth in <400>8 or a nucleotide sequence capable of hybridising to <400>8 under low stringency conditions at 42° C. or a variant, mutant or homologue of said actinomycete
(i) An actinomycete characterised either by a nucleotide sequence corresponding to the nucleotide sequence substantially as set forth in <400>9 or a nucleotide sequence capable of hybridising to <400>9 under low stringency conditions at 42° C. or a variant, mutant or homologue of said actinomycete.
(j) An actinomycete characterised either by a nucleotide sequence corresponding to the nucleotide sequence substantially as set forth in <400>10 or a nucleotide sequence capable of hybridising to <400>10 under low stringency conditions at 42° C. or a variant, mutant or homologue of said actinomycete.
(k) An actinomycete characterised either by a nucleotide sequence corresponding to the nucleotide sequence substantially as set forth in <400>11 or a nucleotide sequence capable of hybridising to <400>11 under low stringency conditions at 42° C. or a variant, mutant or homologue of said actinomycete.
(l) An actinomycete characterised either by a nucleotide sequence corresponding to the nucleotide sequence substantially as set forth in <400>12 or a nucleotide sequence capable of hybridising to <400>12 under low stringency conditions at 42° C. or a variant, mutant or homologue of said actinomycete.
(m) An actinomycete characterised either by a nucleotide sequence corresponding to the nucleotide sequence substantially as set forth in <400>13 or a nucleotide sequence capable of hybridising to <400>13 under low stringency conditions at 42° C. or a variant, mutant or homologue of said actinomycete.
(n) An actinomycete characterised either by a nucleotide sequence corresponding to the nucleotide sequence substantially as set forth in <400>14 or a nucleotide sequence capable of hybridising to <400>14 under low stringency conditions at 42° C. or a variant, mutant or homologue of said actinomycete.

(o) An actinomycete characterised either by a nucleotide sequence corresponding to the nucleotide sequence substantially as set forth in <400>15 or a nucleotide sequence capable of hybridising to <400>15 under low stringency conditions at 42° C. or a variant, mutant or homologue of said actinomycete.
(p) An actinomycete characterised either by a nucleotide sequence corresponding to the nucleotide sequence substantially as set forth in <400>16 or a nucleotide sequence capable of hybridising to <400>16 under low stringency conditions at 42° C. or a variant, mutant or homologue of said actinomycete.
(q) An actinomycete characterised either by a nucleotide sequence corresponding to the nucleotide sequence substantially as set forth in <400>17 or a nucleotide sequence capable of hybridising to <400>17 under low stringency conditions at 42° C. or a variant, mutant or homologue of said actinomycete.
(r) An actinomycete characterised either by a nucleotide sequence corresponding to the nucleotide sequence substantially as set forth in <400>18 or a nucleotide sequence capable of hybridising to <400>18 under low stringency conditions at 42° C. or a variant, mutant or homologue of said actinomycete.
(s) An actinomycete characterised either by a nucleotide sequence corresponding to the nucleotide sequence substantially as set forth in <400>19 or a nucleotide sequence capable of hybridising to <400>19 under low stringency conditions at 42° C. or a variant, mutant or homologue of said actinomycete.
(t) An actinomycete characterised either by a nucleotide sequence corresponding to the nucleotide sequence substantially as set forth in <400>20 or a nucleotide sequence capable of hybridising to <400>20 under low stringency conditions at 42° C. or a variant, mutant or homologue of said actinomycete.
(u) An actinomycete characterised either by a nucleotide sequence corresponding to the nucleotide sequence substantially as set forth in <400>21 or a nucleotide sequence capable of hybridising to <400>21 under low stringency conditions at 42° C. or a variant, mutant or homologue of said actinomycete.
(v) An actinomycete characterised either by a nucleotide sequence corresponding to the nucleotide sequence substantially as set forth in <400>22 or a nucleotide sequence capable of hybridising to <400>22 under low stringency conditions at 42° C. or a variant, mutant or homologue of said actinomycete.
(w) An actinomycete characterised either by a nucleotide sequence corresponding to the nucleotide sequence substantially as set forth in <400>23 or a nucleotide sequence capable of hybridising to <400>23 under low stringency conditions at 42° C. or a variant, mutant or homologue of said actinomycete.
(x) An actinomycete characterised either by a nucleotide sequence corresponding to the nucleotide sequence substantially as set forth in <400>24 or a nucleotide sequence capable of hybridising to <400>24 under low stringency conditions at 42° C. or a variant, mutant or homologue of said actinomycete.
(y) An actinomycete characterised either by a nucleotide sequence corresponding to the nucleotide sequence substantially as set forth in <400>25 or a nucleotide sequence capable of hybridising to <400>25 under low stringency conditions at 42° C. or a variant, mutant or homologue of said actinomycete.
(z) An actinomycete characterised either by a nucleotide sequence corresponding to the nucleotide sequence substantially as set forth in <400>26 or a nucleotide sequence capable of hybridising to <400>26 under low stringency conditions at 42° C. or a variant, mutant or homologue of said actinomycete.
(aa) An actinomycete characterised either by a nucleotide sequence corresponding to the nucleotide sequence substantially as set forth in <400>27 or a nucleotide sequence capable of hybridising to <400>27 under low stringency conditions at 42° C. or a variant, mutant or homologue of said actinomycete.
(ab) An actinomycete characterised either by a nucleotide sequence corresponding to the nucleotide sequence substantially as set forth in <400>28 or a nucleotide sequence capable of hybridising to <400>28 under low stringency conditions at 42° C. or a variant, mutant or homologue of said actinomycete.
(ac) An actinomycete characterised either by a nucleotide sequence corresponding to the nucleotide sequence substantially as set forth in <400>29 or a nucleotide sequence capable of hybridising to <400>29 under low stringency conditions at 42° C. or a variant, mutant or homologue of said actinomycete.
(ad) An actinomycete characterised either by a nucleotide sequence corresponding to the nucleotide sequence substantially as set forth in <400>30 or a nucleotide sequence capable of hybridising to <400>30 under low stringency conditions at 42° C. or a variant, mutant or homologue of said actinomycete.

Preferably, the subject actinomycete is characterised by a nucleotide sequence which has at least about 45% similarity to all or part of the nucleotide sequence indicated by the nucleotide sequence identification numbers detailed above. More preferably, said similarity is 50%, still more preferably 55%, even more preferably 60%, still more preferably 65%, 70%, 80%, 90%, 91%, 92%, 93%, 94%, 95%, 96%, 96.5%, 97%, 97.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or higher.

In accordance with the preceding embodiments and aspect of the invention, still more preferably:
(i) In one embodiment, the actinomycete is characterized by a nucleotide sequence substantially as set forth in <400>12 or a nucleotide sequence with at least 95% identity thereto and wherein said isolate is not Streptomyces caviscabies or Streptomyces setonii.
(ii) In another preferred embodiment, the actinomycete is characterized by a nucleotide sequence substantially as set forth in <400>12 or a nucleotide sequence with at least 95% identity thereto and wherein the actinomycete is classified as Streptomyces triticum as defined in Example 3.
(iii) In another embodiment, the actinomycete comprises the spore coloration of any one of isolates EN19, EN27, EN35, EN57, EN28, SE1, SE2, PM40, PM41, PM228, PM36, PM87, PM252 or PM 171 as set forth in Table 4.
(iv) In another embodiment, the actinomycete comprises the carbohydrate utilization of any one of isolates EN19, EN27, EN35, EN57, EN28, SE1, SE2, PM40, PM41, PM228, PM36, PM87, PM252 or PM 171 as set forth in Table 4.
(v) In a particularly preferred embodiment, the actinomycete is able to utilize sucrose as a sole carbon source.
(vi) In a yet another embodiment, the actinomycete comprises the soluble pigmentation profile of any one of isolates EN19, EN27, EN35, EN57, EN28, SE1, SE2, PM40, PM41, PM228, PM36, PM87, PM252 or PM 171 as set forth in Table 5.
(vii) In a particularly preferred embodiment, the actinomycete produces a light brown, brown, dark brown or black pigment on either ISP5 or ISP7 media. In a yet more preferred embodiment, the actinomycete is able to produce melanin.
(viii) In a yet another embodiment, the actinomycete comprises the biochemical analysis profile of any one of isolates EN19, EN27, EN35, EN57, EN28, SE1, SE2, PM40, PM41, PM228, PM36, PM87, PM252 or PM 171 as set forth in Table 5.
(ix) In another preferred embodiment, the actinomycete is characterized by a nucleotide sequence substantially as set forth in <400>7 or a nucleotide sequence with at least 95% identity thereto and wherein the actinomycete may be classified as a Streptomyces triticum var. griseoviride as defined in Example 3.
(x) In one embodiment, the actinomycete comprises the spore coloration of any one of isolates EN16, EN17, PM144, PM185, PM208, PM342 as set forth in Table 6.
(xi) In another embodiment, the actinomycete comprises the carbohydrate utilization of any one of isolates EN16, EN17, PM144, PM185, PM208, PM342 as set forth in Table 6.
(xii) In a particularly preferred embodiment, the actinomycete is able to utilize sucrose as a sole carbon source.
(xiii) In a yet another embodiment, the actinomycete comprises the soluble pigmentation profile of any one of isolates EN16, EN17, PM144, PM185, PM208, PM342 as set forth in Table 7.
(xiv) In a yet another embodiment, the actinomycete comprises the biochemical analysis profile of any one of isolates EN16, EN17, PM144, PM185, PM208, PM342 as set forth in Table 7.

Reference to “at least 95%” or “more than 95%” identity includes reference to at least 95%, 95.1%, 95.2%, 95.3%, 95.4%, 95.5%, 95.6%, 95.7%, 95.8%, 95.9%, 96%, 96.1%, 96.2%, 96.3%, 96.4%, 96.5%, 96.6%, 96.7%, 96.8%, 96.9%, 97%, 97.1%, 97.2%, 97.3%, 97.4%, 97.5%, 97.6%, 97.7%, 97.8%, 97.9%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.9% and 100%.

Most preferably, the subject actinomycete is selected from the following list of isolates:

| | | | | | | | |
|---|---|---|---|---|---|---|---|
| (a) | EN2 | (b) | EN3 | (c) | EN5 | (d) | EN6 |
| (e) | EN7 | (f) | EN9 | (g) | EN16 | (h) | EN17 |
| (i) | EN19 | (j) | EN23 | (k) | EN26 | (l) | EN27 |
| (m) | EN28 | (n) | EN35 | (o) | EN39 | (p) | EN46 |
| (q) | EN57 | (r) | EN60 | (s) | SE1 | (t) | SE2 |
| (u) | PM36 | (v) | PM40 | (w) | PM41 | (x) | PM87 |
| (y) | PM144 | (z) | PM171 | (aa) | PM185 | (ab) | PM208 |
| (ac) | PM228 | (ad) | PM252 | (ae) | PM342 | | |

Reference to the subject actinomycete being “characterised” by the subject nucleotide sequence should be understood to mean that the subject nucleotide sequence forms part of the nucleic acid composition of the actinomycete. The subject nucleotide sequence may be located at any intracellular location such as on the actinomycete chromosome or at a non-chromosomal location, such as the ribosome. Preferably, the nucleotide sequence comprises part of the gene encoding the 16S species of the small sub-unit of the actinomycetes ribosomal RNA.

Reference herein to a low stringency includes and encompasses from at least about 0% v/v to at least about 15% v/v formamide and from at least about 1M to at least about 2M salt for hybridisation, and at least about 1M to at least about 2M salt for washing conditions. Alternative stringency conditions may be applied where necessary, such as medium stringency, which includes and encompasses from at least about 16% v/v to at least about 30% v/v formamide and from at least about 0.5M to at least about 0.9M salt for hybridisation, and at least about 0.5M to at least about 0.9M salt for washing conditions, or high stringency, which includes and encompasses from at least about 31% v/v to at least about 50% v/v formamide and from at least about 0.01M to at least about 0.15M salt for hybridisation, and at least about 0.01M to at least about 0.15M salt for washing conditions. Stringency may be measured using a range of temperature such as from about 40EC to about 65EC. Particularly useful stringency conditions are at 42EC. In general, washing is carried out at Tₘ=69.3+0.41 (G+C) % [19]=−121C. However, the Tₘ of a duplex DNA decreases by 11C with every increase of 1% in the number of mismatched based pairs (Bonner et al (1973) J. Mol. Biol, 81:123).

The term “similarity” as used herein includes exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. The percentage similarity may be greater than 50% such as at least 70%, 75%, 80%, 85%, 86%, 87%, 88%, 89%, 90%, 91%, 92%, 93%, 94%, 95%, 96%, 97%, 98%, 99% or higher.

To determine the percent identity of two amino acid sequences or of two nucleic acids, the sequences may be aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions can then be compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e. % identity=# of identical positions/total # of overlapping positions×100). Preferably, the two sequences are the same length. The determination of percent identity or homology between two sequences can be accomplished using a mathematical algorithm. A suitable, mathematical algorithm utilized for the comparison of two sequences is the algorithm of Karlin and Altschul (1990) Proc. Natl. Acad. Sci, USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to the nucleic acid molecules of the invention. BLAST protein searches can be performed with XBLAST program, score=50, wordlength=3 to obtain amino acid sequences homologous to the protein molecules of the invention. To obtain gapped alignments for comparison purposes, Gapped BLAST can be utilized as described in Altschul et al. (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov. Another example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, only exact matches are counted.

In another preferred embodiment, the subject stringency conditions are moderate and in yet another preferred embodiment are high.

A “variant” or “mutant” of the subject actinomycete should be understood to mean a microorganism which exhibits at least some of the functional activity of the actinomycete of which it is a variant or mutant. The variation or mutation characterising such an actinomycete may take any form including a genetic or a non-genetic variation or mutation. The subject variation or mutation may be naturally or non-naturally occurring. By “homologue” is meant that the microorganism utilised in the method of the present invention is of a species or genera other than that defined. This may occur, for example, where it is determined that an actinomycete of another species exhibits the same functional characteristics and colonisation properties as the actinomycete of interest.

Derivatives of the metabolite defined herein include fragments, parts, portions, mutants, variants and mimetics from natural, synthetic or recombinant sources including fusion proteins. Parts or fragments include, for example, active regions of the metabolite. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterised by the removal of one or more amino acids from the sequence. Substitutional amino acid variants are those in which at least one residue in the sequence has been removed and a different residue inserted in its place. An example of substitutional amino acid variants are conservative amino acid substitutions. Conservative amino acid substitutions typically include substitutions within the following groups: glycine and alanine; valine, isoleucine and leucine; aspartic acid and glutamic acid; asparagine and glutamine; serine and threonine; lysine and arginine; and phenylalanine and tyrosine. Additions to amino acid sequences including fusions with other peptides, polypeptides or proteins.

Reference to “derivatives” of metabolites also includes reference to small molecular weight, non-peptide, organic compound molecules.

Chemical and functional equivalents of the metabolite should be understood as molecules exhibiting any one or more of the functional activities of the metabolite and may be derived from any source such as being chemically synthesized or identified via screening processes such as natural product screening.

Derivatives of the metabolite include fragments having particular epitopes or parts of the entire metabolite fused to peptides, polypeptides or other proteinaceous or non-proteinaceous molecules.

Analogues of the metabolite contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogues.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acid contemplated herein is shown in Table 1.

TABLE 1

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl--aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | | |
| ethylamino)cyclopropan | | | |

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

Actinomycetes may be introduced to the plant or its propagation material by any suitable means. It should be understood that reference to “actinomycete” includes reference to both the bacterium itself, the spore of the bacterium or the mycelium of the bacterium. Examples of means by which actinomycetes can be introduced to the plant include, but are not limited to:
(i) treatment of seeds with a spore or mycelial or cellular preparation of the actinomycete of interest.
(ii) treatment of seeds with the actinomycete derived metabolite of interest.
(iii) treatment of plants with the spore or bacterial preparation of the actinomycete of interest.
(iv) treatment of plants with the actinomycete derived metabolite of interest.
(v) treatment of seeds with actinomycete, or actinomycetes, of interest, together with actinomycete derived metabolite or metabolites of interest.
(vi) incorporation into soil of the actinomycete or actinomycetes of interest as a bacterial suspension either at the time of sowing, or prior to or after sowing.
(vii) incorporation into soil of the actinomycete-derived metabolites of interest as a solution or suspensions either at the time of sowing, or prior to or after sowing.
(viii) incorporation into soil of the actinomycete or actinomycetes of interest as a bacterial suspensions, together with actinomycete-derived metabolite or metabolites of interest, either at the time of sowing, or prior to, or after sowing.
(ix) incorporation into soil of the actinomycete or actinomycetes of interest as a powder or pellet adjacent to the seed either at the time of sowing, or prior to, or after sowing.
(x) incorporation into soil of the actinomycete derived metabolite or metabolites of interest as a powder or pellet adjacent to the seed either at the time of sowing or prior to, or after sowing.
(xi) incorporation into soil of the actinomycete or actinomycetes of interest, together with actinomycete-derived metabolite or metabolites of interest, as a powder or pellet adjacent to the seed either at the time of sowing, or prior to, or after sowing.

It is within the skill of the person of skill in the art to determine both the most appropriate time point (in terms of crop cultivation) at which to apply the method of the present invention and the most suitable means of introducing the subject actinomycete to the plant in terms of both route of administration and appropriate formulation of actinomycete or metabolite thereof. For example, where the treatment is intended to be utilised as a prophylactic bio-control agent it may be most appropriate to pre-treat seeds prior to germination, planting and cultivation. However, where it is desired to utilise the present invention in order to minimise the detrimental impact of an existing pathogenic infection, it may be necessary to treat the plant itself.

Reference to “effective number” or “effective amount” means that number or amount necessary to at least partly attain the desired effect (for example growth promoting activity or bio-control activity), or to delay the onset of, inhibit the progression of, or halt altogether the onset or progression of the particular agricultural condition being treated. Such amounts will depend, of course, on the particular situation, the severity of the condition (for example the severity of infection or likely infection or the severity of growth related defects) and the individual crop parameters including its physical condition, stage of germination and any other concurrent treatments which are being applied (such as other bio-control agents or fertilisers). These factors are well known to those of ordinary skill in the art and can be addressed with no more than routine experimentation. It is generally preferred that an optimum dose of actinomycete formulation be used, that is, the highest safe dose according to sound agricultural judgment. It will be understood by those of ordinary skill in the art, however, that a lower dose or tolerable dose may be administered for any other reason.

Without limiting the invention to any one theory or mode of action, it is thought that the actinomycetes of the present invention will establish an endophytic existence within the root system of the subject plant. However, it should nevertheless be understood that endophytic bacteria are known to coexist with the plant at other locations such as in the leaves or stems.

The inventors have determined that the endophytic actinomycetes disclosed herein exhibit one or more functional activities which lead to improvement in the productivity of the plant with which an endophytic relationship has been established. In particular, the inventors have determined that the actinomycete isolates defined as EN2, EN3, EN9, EN16, EN17, EN19, EN23, EN26, EN27, EN28, EN35, EN39, EN46, EN57, EN60, SE1 and PM87 function as bio-control agents. Further, the inventors have determined that the pathogens in respect of which bio-control is provided include Gaeumannomyces graminis var. tritici, Pythium spp. and Rhizoctonia solani, Fusarium sp., aphids and a range of insect and nematode pests.

The inventors have also determined that actinomycete isolates EN2, EN3, EN6, EN9, EN16, EN27, EN57, EN60, SE1, SE2, PM87, PM185 and PM208 induce growth promotion activity and, in particular, induce germination promotion. Finally, it has been determined that EN2, EN3, EN9, EN16, EN23, EN27, EN28, EN35, EN46, EN57, EN60, SE1, SE2 and PM87 exhibit both growth promotion and bio-control activity. Still further, it has been determined that some of the actinomycete strains detailed herein produce high levels of the plant growth hormone idole acetic acid while some strains induce genes related to Induced Systemic Resistance in plants. Without limiting the present invention in any way some of the strains disclosed herein exhibit borth properties.

Accordingly, in a most preferred embodiment there is provided a method of improving cereal plant productivity said method comprising introducing into said cereal plant or propagation material thereof:
(i) an effective number of actinomycetes selected from EN2, EN3, EN5, EN6, EN7, EN9, EN16, EN17, EN19, EN23, EN26, EN27, EN28, EN35, EN39, EN46, EN57, EN60, SE1, SE2, PM36, PM40, PM41, PM87, PM144, PM171, PM185, PM208, PM228, PM252 AND PM342 or variants, mutants or homologues thereof; and/or
(ii) an effective amount of one or more metabolites derived from the actinomycetes of (i) or derivative, homologue, analogue, chemical equivalent or mimetic thereof;
   for a time and under conditions sufficient to induce in the subject cereal plant bio-control activity.

Preferably, said bio-control activity is bio-control in relation to Gaeumannomyces graminis var. tritici, Pythium spp., Rhizoctonia solani or Fusarium sp.

In another most preferred embodiment there is provided the method of improving cereal plant productivity said method comprising introducing into said cereal plant or propagation material thereof:

(i) an effective number of actinomycetes selected from EN2, EN3, EN6, EN9, EN16, EN27, EN57, EN60, SE1, SE2, PM87, PM185 and PM208 or variants, mutants or homologues thereof; and/or

(ii) an effective amount of one or more metabolites derived from the actinomycetes of (i) or derivative, homologue, analogue, chemical equivalent or mimetic thereof;

for a time and under conditions sufficient to induce in the subject cereal plant growth promotion.

Preferably, said growth promotion is early growth vigour, grain yield increases and/or germination promotion.

In still yet another most preferred embodiment there is provided the method for improving cereal plant productivity said method comprising introducing into said cereal plant or propagation material thereof:
(i) an effective number of actinomycetes selected from EN2, EN3, EN9, EN16, EN23, EN27, EN28, EN35, EN46, EN57, EN60, SE1, SE2 and PM87 or variants, mutants or homologues thereof; and/or
(ii) an effective amount of one or more metabolites derived from the actinomycetes of (i) or derivative, homologue, analogue, chemical equivalent or mimetic thereof;
   for a time and under conditions sufficient to induce in a subject plant both growth promoting activity and bio-control activity.

In yet another most preferred embodiment there is provided a method of improving cereal plant productivity said method comprising introducing into said cereal plant productivity said method comprising introducing into said cereal plant or propagation material thereof:
(iii) an effective number of actinomycetes selected from EN2, EN3, EN5, EN16, EN17, EN19, EN23, EN27, EN28, EN35, EN46, EN57, PM36, PM40, PM41, PM87, PM110, PM119, PM144, PM171, PM185, PM208, PM228, PM252, PM342, SE1 and SE2 or variants, mutants or homologues thereof; and/or
(iv) an effective amount of one or more metabolites derived from the actinomycetes of (i) or derivative, homologue, analogue, chemical equivalent or mimetic thereof;
   for a time and under conditions sufficient to induce in the subject cereal plant bio-control activity.

Preferably said bio-control activity is bio-control in relation to aphids.

In accordance with these preferred embodiments, said cereal plant is preferably wheat, barley, maize, rye, triticale, oats, canary seed, sorghum, millet or rice.

As detailed hereinbefore, the inventors of the present invention have isolated several novel species of endophytic actinomycetes.

Accordingly, another aspect of the present invention is directed to a method of improving plant productivity said method comprising introducing into said plant or propagation materials thereof:
(i) an effective number of novel endophytic actinomycetes or variants, mutants or homologues thereof; and/or
(ii) an effective amount of one or more metabolites derived from the actinomycetes of (i) or derivative, homologue, analogue, chemical equivalent or mimetic thereof;
   for a time and under conditions sufficient to induce in the subject plant at least one characteristic of improved productivity.

Preferably, said novel endophytic actinomycete is selected from the list consisting of:
(a) An actinomycete characterised either by a nucleotide sequence corresponding to the nucleotide sequence substantially as set forth in <400>1 or a nucleotide sequence capable of hybridising to <400>1 under low stringency conditions at 42° C. or a variant, mutant or homologue thereof.
(b) An actinomycete characterised either by nucleotide sequence corresponding to the nucleotide sequence substantially as set forth in <400>2 or a nucleotide sequence capable of hybridising to <400>2 under low stringency conditions at 42° C. or a variant, mutant or homologue thereof.
(c) An actinomycete characterised either by nucleotide sequence corresponding to the nucleotide sequence substantially as set forth in <400>7 or a nucleotide sequence capable of hybridising to <400>7 under low stringency conditions at 42° C. or a variant, mutant or homologue thereof.
(d) An actinomycete characterised either by nucleotide sequence corresponding to the nucleotide sequence substantially as set forth in <400>10 or a nucleotide sequence capable of hybridising to <400>10 under low stringency conditions at 42° C. or a variant, mutant or homologue thereof.
(e) An actinomycete characterised either by nucleotide sequence corresponding to the nucleotide sequence substantially as set forth in <400>12 or a nucleotide sequence capable of hybridising to <400>12 under low stringency conditions at 42° C. or a variant, mutant or homologue thereof.
(f) An actinomycete characterised either by nucleotide sequence corresponding to the nucleotide sequence substantially as set forth in <400>13 or a nucleotide sequence capable of hybridising to <400>13 under low stringency conditions at 42° C. or a variant, mutant or homologue thereof.
(g) An actinomycete characterised either by nucleotide sequence corresponding to the nucleotide sequence substantially as set forth in <400>16 or a nucleotide sequence capable of hybridising to <400>16 under low stringency conditions at 42° C. or a variant, mutant or homologue thereof.
(h) An actinomycete characterised either by nucleotide sequence corresponding to the nucleotide sequence substantially as set forth in <400>18 or a nucleotide sequence capable of hybridising to <400>18 under low stringency conditions at 42° C. or a variant, mutant or homologue thereof.
(i) An actinomycete characterised either by nucleotide sequence corresponding to the nucleotide sequence substantially as set forth in <400>24 or a nucleotide sequence capable of hybridising to <400>24 under low stringency conditions at 42° C. or a variant, mutant or homologue thereof.

In yet another most preferred embodiment said actinomycete corresponds to EN2, EN3, EN16, EN23, EN27, EN28, EN46, EN60 or PM87.

In another preferred embodiment, said novel endophytic actinomycete is selected from the list consisting of:
(a) An actinomycete characterised either by nucleotide sequence corresponding to the nucleotide sequence substantially as set forth in <400>3 or a nucleotide sequence capable of hybridising to <400>3 under low stringency conditions at 42° C. or a variant, mutant or homologue thereof.
(b) An actinomycete characterised either by nucleotide sequence corresponding to the nucleotide sequence substantially as set forth in <400>4 or a nucleotide sequence capable of hybridising to <400>4 under low stringency conditions at 42° C. or a variant, mutant or homologue thereof.
(c) An actinomycete characterised either by nucleotide sequence corresponding to the nucleotide sequence substantially as set forth in <400>5 or a nucleotide sequence capable of hybridising to <400>5 under low stringency conditions at 42° C. or a variant, mutant or homologue thereof.
(d) An actinomycete characterised either by nucleotide sequence corresponding to the nucleotide sequence substantially as set forth in <400>6 or a nucleotide sequence capable of hybridising to <400>6 under low stringency conditions at 42° C. or a variant, mutant or homologue thereof.
(e) An actinomycete characterised either by nucleotide sequence corresponding to the nucleotide sequence substantially as set forth in <400>8 or a nucleotide sequence capable of hybridising to <400>8 under low stringency conditions at 42° C. or a variant, mutant or homologue thereof.
(f) An actinomycete characterised either by nucleotide sequence corresponding to the nucleotide sequence substantially as set forth in <400>9 or a nucleotide sequence capable of hybridising to <400>9 under low stringency conditions at 42° C. or a variant, mutant or homologue thereof.
(g) An actinomycete characterised either by nucleotide sequence corresponding to the nucleotide sequence substantially as set forth in <400>11 or a nucleotide sequence capable of hybridising to <400>11 under low stringency conditions at 42° C. or a variant, mutant or homologue thereof.
(h) An actinomycete characterised either by nucleotide sequence corresponding to the nucleotide sequence substantially as set forth in <400>14 or a nucleotide sequence capable of hybridising to <400>14 under low stringency conditions at 42° C. or a variant, mutant or homologue thereof.
(i) An actinomycete characterised either by nucleotide sequence corresponding to the nucleotide sequence substantially as set forth in <400>15 or a nucleotide sequence capable of hybridising to <400>15 under low stringency conditions at 42° C. or a variant, mutant or homologue thereof.
(j) An actinomycete characterised either by nucleotide sequence corresponding to the nucleotide sequence substantially as set forth in <400>17 or a nucleotide sequence capable of hybridising to <400>17 under low stringency conditions at 42° C. or a variant, mutant or homologue thereof.
(k) An actinomycete characterised either by nucleotide sequence corresponding to the nucleotide sequence substantially as set forth in <400>19 or a nucleotide sequence capable of hybridising to <400>19 under low stringency conditions at 42° C. or a variant, mutant or homologue thereof.
(l) An actinomycete characterised either by nucleotide sequence corresponding to the nucleotide sequence substantially as set forth in <400>20 or a nucleotide sequence capable of hybridising to <400>20 under low stringency conditions at 42° C. or a variant, mutant or homologue thereof.
(v) An actinomycete characterised either by nucleotide sequence corresponding to the nucleotide sequence substantially as set forth in <400>21 or a nucleotide sequence capable of hybridising to <400>21 under low stringency conditions at 42° C. or a variant, mutant or homologue thereof.
(w) An actinomycete characterised either by nucleotide sequence corresponding to the nucleotide sequence substantially as set forth in <400>22 or a nucleotide sequence capable of hybridising to <400>22 under low stringency conditions at 42° C. or a variant, mutant or homologue thereof.
(x) An actinomycete characterised either by nucleotide sequence corresponding to the nucleotide sequence substantially as set forth in <400>23 or a nucleotide sequence capable of hybridising to <400>23 under low stringency conditions at 42° C. or a variant, mutant or homologue thereof.
(y) An actinomycete characterised either by nucleotide sequence corresponding to the nucleotide sequence substantially as set forth in <400>25 or a nucleotide sequence capable of hybridising to <400>25 under low stringency conditions at 42° C. or a variant, mutant or homologue thereof.
(z) An actinomycete characterised either by nucleotide sequence corresponding to the nucleotide sequence substantially as set forth in <400>25 or a nucleotide sequence capable of hybridising to <400>25 under low stringency conditions at 42° C. or a variant, mutant or homologue thereof.
(aa) An actinomycete characterised either by nucleotide sequence corresponding to the nucleotide sequence substantially as set forth in <400>26 or a nucleotide sequence capable of hybridising to <400>26 under low stringency conditions at 42° C. or a variant, mutant or homologue thereof.
(bb) An actinomycete characterised either by nucleotide sequence corresponding to the nucleotide sequence substantially as set forth in <400>27 or a nucleotide sequence capable of hybridising to <400>27 under low stringency conditions at 42° C. or a variant, mutant or homologue thereof,
(cc) An actinomycete characterised either by nucleotide sequence corresponding to the nucleotide sequence substantially as set forth in <400>28 or a nucleotide sequence capable of hybridising to <400>28 under low stringency conditions at 42° C. or a variant, mutant or homologue thereof.
(dd) An actinomycete characterised either by nucleotide sequence corresponding to the nucleotide sequence substantially as set forth in <400>29 or a nucleotide sequence capable of hybridising to <400>29 under low stringency conditions at 42° C. or a variant, mutant or homologue thereof.

Preferably, the subject actinomycete is characterised by a nucleotide sequence which has at least 45% similarity to all or part of the nucleotide sequence indicated by the nucleotide sequence identification numbers detailed above. More preferably, said similarity is 50%, still more preferably 55%, even more preferably 60%, still more preferably 65%, 70%, 80%, 90%, 91%, 92%, 93%, 94%, 95%, 96%, 96.5%, 97%, 97.5%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or higher.

In accordance with the preceding embodiments and aspect of the invention, still more preferably:
(i) In one embodiment, the actinomycete is characterized by a nucleotide sequence substantially as set forth in <400>12 or a nucleotide sequence with at least 95% identity thereto and wherein said isolate is not Streptomyces caviscabies or Streptomyces setonii.
(ii) In another preferred embodiment, the actinomycete is characterized by a nucleotide sequence substantially as set forth in <400>12 or a nucleotide sequence with at least 95% identity thereto and wherein the actinomycete is classified as Streptomyces triticum as defined in Example 3.
(iii) In another embodiment, the actinomycete comprises the spore coloration of any one of isolates EN19, EN27, EN35, EN57, EN28, SE1, SE2, PM40, PM41, PM228, PM36, PM87, PM252 or PM 171 as set forth in Table 4.
(iv) In another embodiment, the actinomycete comprises the carbohydrate utilization of any one of isolates EN19, EN27, EN35, EN57, EN28, SE1, SE2, PM40, PM41, PM228, PM36, PM87, PM252 or PM 171 as set forth in Table 4.
(v) In a particularly preferred embodiment, the actinomycete is able to utilize sucrose as a sole carbon source.
(vi) In a yet another embodiment, the actinomycete comprises the soluble pigmentation profile of any one of isolates EN19, EN27, EN35, EN57, EN28, SE1, SE2, PM40, PM41, PM228, PM36, PM87, PM252 or PM 171 as set forth in Table 5.
(vii) In a particularly preferred embodiment, the actinomycete produces a light brown, brown, dark brown or black pigment on either ISP5 or ISP7 media. In a yet more preferred embodiment, the actinomycete is able to produce melanin.
(viii) In a yet another embodiment, the actinomycete comprises the biochemical analysis profile of any one of isolates EN19, EN27, EN35, EN57, EN28, SE1, SE2, PM40, PM41, PM228, PM36, PM87, PM252 or PM 171 as set forth in Table 5.
(ix) In another preferred embodiment, the actinomycete is characterized by a nucleotide sequence substantially as set forth in <400>7 or a nucleotide sequence with at least 95% identity thereto and wherein the actinomycete may be classified as a Streptomyces triticum var. griseoviride as defined in Example 3.
(x) In one embodiment, the actinomycete comprises the spore coloration of any one of isolates EN16, EN17, PM144, PM185, PM208, PM342 as set forth in Table 6.
(xi) In another embodiment, the actinomycete comprises the carbohydrate utilization of any one of isolates EN16, EN17, PM144, PM185, PM208, PM342 as set forth in Table 6.

(xii) In a particularly preferred embodiment, the actinomycete is able to utilize sucrose as a sole carbon source.

(xiii) In a yet another embodiment, the actinomycete comprises the soluble pigmentation profile of any one of isolates EN16, EN17, PM144, PM185, PM208, PM342 as set forth in Table 7.

(xiv) In a yet another embodiment, the actinomycete comprises the biochemical analysis profile of any one of isolates EN16, EN17, PM144, PM185, PM208, PM342 as set forth in Table 7.

Reference to “at least 95%” or “more than 95%” identity includes reference to at least 95%, 96%, 97%, 98%, 98.1%, 98.2%, 98.3%, 98.4%, 98.5%, 98.6%, 98.7%, 98.9%, 99%, 99.1%, 99.2%, 99.3%, 99.4%, 99.5%, 99.6%, 99.7%, 99.9% and 100%.

In yet another most preferred embodiment, said actinomycete corresponds to EN5, EN6, EN7, EN9, EN17, EN19, EN26, EN35, EN39, EN57, SE1, SE2, PM36, PM40, PM41, PM171, PM185, PM208, PM228, PM252, PM342 or PM144.

As described hereinbefore, although the preferred aspects of the present invention are to introduce a single species of actinomycete into a plant in order to achieve productivity improvements, the present invention also encompasses the administration of two or more species of actinomycete into any given plant. In this regard, the inventors have determined that particularly effective actinomycete combinations for use in the method of the present invention include:

(i) EN2 and EN9 and EN23

(ii) EN9 and EN27 and EN28

(ii) EN39 and EN46

However, it should be understood that the method of the present invention extends to any other suitable combination of actinomycetes, which combination can be identified without undue experimentation based on the teachings provided herein.

In still yet another aspect the present invention is directed to the cereal plant-derived endophytic actinomycetes or variants, mutants or homologues thereof or metabolites derived therefrom or derivatives, homologues, analogues, chemical equivalents or mimetics thereof for use in the method of the present invention.

In yet still another aspect there is provided an agricultural composition comprising the endophytic actinomycetes hereinbefore described or metabolites derived therefrom together with one or more agriculturally acceptable carriers and/or diluents. Preparation of said agricultural compositions would be known to those of skill in the art.

As detailed hereinbefore, the inventors have surprisingly identified novel species of actinomycetes, which actinomycetes were identified due to their co-existence in an endophytic relationship with cereal plants, such as wheat plants. These actinomycetes, and the metabolites produced therefrom, are useful in a range of applications including, but not limited to, agricultural applications (such as growth promotion or bio-control activity), biodegradation and therapeutic or prophylactic medical treatments for animals or humans.

Accordingly, another aspect of the present invention is directed to a novel, isolated plant-derived endophytic actinomycete or variant, mutant or homologue thereof.

More particularly, the present invention is directed to a novel, isolated cereal plant-derived endophytic actinomycete or variant, mutant or homologue thereof.

Reference to “plant” and “cereal plant-derived endophytic actinomycete” should be understood to have the same meaning as hereinbefore defined. In this regard, the subject cereal plant is preferably a wheat plant.

Accordingly, the present invention still more particularly provides a novel, isolated wheat plant-derived endophytic actinomycete or variant, mutant or homologue thereof.

It should be understood that the isolated actinomycete according to this aspect of the present invention is defined as “endophytic” on the basis that in appropriate circumstances, it can co-exist with a plant in an endophytic relationship. However, it should be understood that the subject actinomycete may exhibit a range of characteristics including, inter alia, the ability to exist in the rhizosphere. Further, although the actinomycete may exhibit the ability to co-exist in an endophytic relationship with a cereal plant, it may be that such co-existence is only possible with some members of a particular family of plants but not all members. This may be due, for example, to characteristics which are inherent in the host plant.

It should also be understood that although the novel actinomycete is defined by reference to it being “plant-derived”, this is a reference merely to the fact that these novel actinomycetes have been identified in the defined plant, but not that the isolated actinomycetes falling within the scope of this invention are necessarily isolated directly from a plant. For example, it may be that the subject actinomycetes, although originally identified in a cereal plant, are isolated from ongoing in vitro cell cultures. Alternatively, it may be that the subject actinomycetes are also found in non-plant sources, such as in the rhizosphere, and can be isolated from these non-plant sources.

By “isolated” is meant that the actinomycete has undergone at least one step of purification from a biological source. Preferably, the actinomycete is also pure meaning that a composition comprises at least about 20%, more preferably at least about 40%, still more preferably at least about 65%, even still more preferably at least about 80-90% or greater of the actinomycete as determined by weight, activity or other convenient means, relative to other compounds in the composition.

The inventors have characterised the subject actinomycetes based on their 16S rDNA sequences and have determined that the actinomycetes comprising isolates EN2, EN3, EN5, EN6, EN7, EN9, EN16, EN17, EN19, EN23, EN26, EN27, EN28, EN35, EN39, EN46, EN57, EN60, SE1, SE2, PM36, PM40, PM41, PM87, PM144, PM171, PM185, PM208, PM228, PM252 and PM342 correspond to previously unidentified species of actinomycetes. These isolates correspond to populations of actinomycetes comprising the rDNA sequence substantially as set forth in the nucleic acid sequences detailed earlier.

Accordingly, in one aspect, the present invention provides an isolated actinomycete wherein said actinomycete is characterised either by a nucleotide sequence corresponding to the nucleotide sequence substantially as set forth in <400>1 or a nucleotide sequence capable of hybridising to <400>1 under low stringency conditions at 42° C. or a variant, mutant or homologue of said actinomycete.

Preferably, the subject actinomycete corresponds to EN2 (AGAL Deposit No. NM03/35895).

Without limiting the present invention to any one theory or mode of action, EN2 is thought to correspond to a new species of Microbispora.

In another aspect the present invention provides an isolated actinomycete wherein said actinomycete is characterised either by a nucleotide sequence corresponding to the nucleotide sequence substantially as set forth in <400>2 or a nucleotide sequence capable of hybridising to <400>2 under low stringency conditions at 42° C. or a variant, mutant or homologue of said actinomycete.

Preferably, the subject actinomycete corresponds to EN3 (AGAL Deposit No. NMO3/36501).

Without limiting the present invention to any one theory or mode of action, EN3 is thought to correspond to a novel Streptomyces species.

In yet another aspect the present invention provides an isolated actinomycete wherein said actinomycete is characterised either by a nucleotide sequence corresponding to the nucleotide sequence substantially as set forth in <400>7 or a nucleotide sequence capable of hybridising to <400>7 under low stringency conditions at 42° C. or a variant, mutant or homologue of said actinomycete.

Preferably, the subject actinomycete corresponds to EN16 (AGAL Deposit No. NM03/35604).

Without limiting the present invention to any one theory or mode of action, EN16 is thought to correspond to a new species termed Streptomyces triticum var griseoviride.

In still another aspect the present invention provides an isolated actinomycete wherein said actinomycete is characterised either by a nucleotide sequence corresponding to the nucleotide sequence substantially as set forth in <400>10 or a nucleotide sequence capable of hybridising to <400>10 under low stringency conditions at 42° C. or a variant, mutant or homologue of said actinomycete.

Preferably, the subject actinomycete corresponds to EN23 (AGAL Deposit No. NM03/35605).

Without limiting the present invention to any one theory or mode of action, EN23 is thought to correspond to a new species termed Streptomyces triticum.

In yet still another aspect the present invention provides an isolated actinomycete wherein said actinomycete is characterised either by a nucleotide sequence corresponding to the nucleotide sequence substantially as set forth in <400>12 or a nucleotide sequence capable of hybridising to <400>12 under low stringency conditions at 42° C. or a variant, mutant or homologue of said actinomycete.

Preferably, the subject actinomycete corresponds to EN27 (AGAL Deposit No. NM03/35606).

Without limiting the present invention to any one theory or mode of action, EN27 is thought to correspond to a new species termed Streptomyces triticum.

In still yet another aspect the present invention provides an isolated actinomycete wherein said actinomycete is characterised either by a nucleotide sequence corresponding to the nucleotide sequence substantially as set forth in <400>13 or a nucleotide sequence capable of hybridising to <400>13 under low stringency conditions at 42° C. or a variant, mutant or homologue of said actinomycete.

Preferably, the subject actinomycete corresponds to EN28 (AGAL Deposit No. NM03/35607).

Without limiting the present invention to any one theory or mode of action, EN28 is thought to correspond to a new species termed Streptomyces triticum.

In yet another further aspect the present invention provides an isolated actinomycete wherein said actinomycete is characterised either by a nucleotide sequence corresponding to the nucleotide sequence substantially as set forth in <400>16 or a nucleotide sequence capable of hybridising to <400>16 under low stringency conditions at 42° C. or a variant, mutant or homologue of said actinomycete.

Preferably, the subject actinomycete corresponds to EN46 (AGAL Deposit No. NM03/35609).

Without limiting the present invention to any one theory or mode of action, EN46 is thought to correspond to Nocardioides albus.

In still another further aspect the present invention provides an isolated actinomycete wherein said actinomycete is characterised either by a nucleotide sequence corresponding to the nucleotide sequence substantially as set forth in <400>18 or a nucleotide sequence capable of hybridising to <400>18 under low stringency conditions at 42° C. or a variant, mutant or homologue of said actinomycete.

Preferably, the subject actinomycete corresponds to EN60 (AGAL Deposit No. NM03/35896).

Without limiting the present invention to any one theory or mode of action, EN60 is thought to correspond to a novel species related to Streptomyces argenteolus.

In yet still another aspect the present invention provides an isolated actinomycete wherein said actinomycete is characterised either by a nucleotide sequence corresponding to the nucleotide sequence substantially as set forth in <400>24 or a nucleotide sequence capable of hybridising to <400>24 under low stringency conditions at 42° C. or a variant, mutant or homologue of said actinomycete.

Preferably, the subject actinomycete corresponds to PM87 (AGAL Deposit No. NM03/35608).

Without limiting the present invention to any one theory or mode of action, PM87 is thought to correspond to a new species termed Streptomyces triticum.

In another aspect the present invention provides an isolated actinomycete wherein said actinomycete is characterised either by a nucleotide sequence corresponding to the nucleotide sequence substantially as set forth in <400>3 or a nucleotide sequence capable of hybridising to <400>3 under low stringency conditions at 42° C. or a variant, mutant or homologue of said actinomycete.

Preferably, the subject actinomycete corresponds to EN5.

In yet another aspect the present invention provides an isolated actinomycete wherein said actinomycete is characterised either by a nucleotide sequence corresponding to the nucleotide sequence substantially as set forth in <400>4 or a nucleotide sequence capable of hybridising to <400>4 under low stringency conditions at 42° C. or a variant, mutant or homologue of said actinomycete.

Preferably, the subject actinomycete corresponds to EN6.

In still another aspect the present invention provides an isolated actinomycete wherein said actinomycete is characterised either by a nucleotide sequence corresponding to the nucleotide sequence substantially as set forth in <400>5 or a nucleotide sequence capable of hybridising to <400>5 under low stringency conditions at 42° C. or a variant, mutant or homologue of said actinomycete.

Preferably, the subject actinomycete corresponds to EN7.

In yet still another aspect the present invention provides an isolated actinomycete wherein said actinomycete is characterised either by a nucleotide sequence corresponding to the nucleotide sequence substantially as set forth in <400>6 or a nucleotide sequence capable of hybridising to <400>6 under low stringency conditions at 42° C. or a variant, mutant or homologue of said actinomycete.

Preferably, the subject actinomycete corresponds to EN9.

In a further aspect the present invention provides an isolated actinomycete wherein said actinomycete is characterised either by a nucleotide sequence corresponding to the nucleotide sequence substantially as set forth in <400>8 or a nucleotide sequence capable of hybridising to <400>8 under low stringency conditions at 42° C. or a variant, mutant or homologue of said actinomycete.

Preferably, the subject actinomycete corresponds to EN17.

In another further aspect the present invention provides an isolated actinomycete wherein said actinomycete is characterised either by a nucleotide sequence corresponding to the nucleotide sequence substantially as set forth in <400>9 or a nucleotide sequence capable of hybridising to <400>9 under low stringency conditions at 42° C. or a variant, mutant or homologue of said actinomycete.

Preferably, the subject actinomycete corresponds to EN19.

In yet another aspect the present invention provides an isolated actinomycete wherein said actinomycete is characterised either by a nucleotide sequence corresponding to the nucleotide sequence substantially as set forth in <400>11 or a nucleotide sequence capable of hybridising to <400>11 under low stringency conditions at 42° C. or a variant, mutant or homologue of said actinomycete.

Preferably, the subject actinomycete corresponds to EN26.

In still another aspect the present invention provides an isolated actinomycete wherein said actinomycete is characterised either by a nucleotide sequence corresponding to the nucleotide sequence substantially as set forth in <400>14 or a nucleotide sequence capable of hybridising to <400>14 under low stringency conditions at 42° C. or a variant, mutant or homologue of said actinomycete.

Preferably, the subject actinomycete corresponds to EN35.

In yet still another aspect the present invention provides an isolated actinomycete wherein said actinomycete is characterised either by a nucleotide sequence corresponding to the nucleotide sequence substantially as set forth in <400>15 or a nucleotide sequence capable of hybridising to <400>15 under low stringency conditions at 42° C. or a variant, mutant or homologue of said actinomycete.

Preferably, the subject actinomycete corresponds to EN39.

In still yet another aspect the present invention provides an isolated actinomycete wherein said actinomycete is characterised either by a nucleotide sequence corresponding to the nucleotide sequence substantially as set forth in <400>17 or a nucleotide sequence capable of hybridising to <400>17 under low stringency conditions at 42° C. or a variant, mutant or homologue of said actinomycete.

Preferably, the subject actinomycete corresponds to EN57.

In another aspect the present invention provides an isolated actinomycete wherein said actinomycete is characterised either by a nucleotide sequence corresponding to the nucleotide sequence substantially as set forth in <400>19 or a nucleotide sequence capable of hybridising to <400>19 under low stringency conditions at 42° C. or a variant, mutant or homologue of said actinomycete.

Preferably, the subject actinomycete corresponds to SE1.

In yet another aspect the present invention provides an isolated actinomycete wherein said actinomycete is characterised either by a nucleotide sequence corresponding to the nucleotide sequence substantially as set forth in <400>20 or a nucleotide sequence capable of hybridising to <400>20 under low stringency conditions at 42° C. or a variant, mutant or homologue of said actinomycete.

Preferably, the subject actinomycete corresponds to SE2.

In still another aspect the present invention provides an isolated actinomycete wherein said actinomycete is characterised either by a nucleotide sequence corresponding to the nucleotide sequence substantially as set forth in <400>21 or a nucleotide sequence capable of hybridising to <400>21 under low stringency conditions at 42° C. or a variant, mutant or homologue of said actinomycete.

Preferably, the subject actinomycete corresponds to PM36.

In yet still another aspect the present invention provides an isolated actinomycete wherein said actinomycete is characterised either by a nucleotide sequence corresponding to the nucleotide sequence substantially as set forth in <400>22 or a nucleotide sequence capable of hybridising to <400>22 under low stringency conditions at 42° C. or a variant, mutant or homologue of said actinomycete.

Preferably, the subject actinomycete corresponds to PM40.

In a further aspect the present invention provides an isolated actinomycete wherein said actinomycete is characterised either by a nucleotide sequence corresponding to the nucleotide sequence substantially as set forth in <400>23 or a nucleotide sequence capable of hybridising to <400>23 under low stringency conditions at 42° C. or a variant, mutant or homologue of said actinomycete.

Preferably, the subject actinomycete corresponds to PM41.

In still another aspect the present invention provides an isolated actinomycete wherein said actinomycete is characterised either by a nucleotide sequence corresponding to the nucleotide sequence substantially as set forth in <400>25 or a nucleotide sequence capable of hybridising to <400>25 under low stringency conditions at 42° C. or a variant, mutant or homologue of said actinomycete.

Preferably, the subject actinomycete corresponds to PM171.

In yet still another aspect the present invention provides an isolated actinomycete wherein said actinomycete is characterised either by a nucleotide sequence corresponding to the nucleotide sequence substantially as set forth in <400>26 or a nucleotide sequence capable of hybridising to <400>26 under low stringency conditions at 42° C. or a variant, mutant or homologue of said actinomycete.

Preferably, the subject actinomycete corresponds to PM185.

In still yet another aspect the present invention provides an isolated actinomycete wherein said actinomycete is characterised either by a nucleotide sequence corresponding to the nucleotide sequence substantially as set forth in <400>27 or a nucleotide sequence capable of hybridising to <400>27 under low stringency conditions at 42° C. or a variant, mutant or homologue of said actinomycete.

Preferably, the subject actinomycete corresponds to PM208.

In another aspect the present invention provides an isolated actinomycete wherein said actinomycete is characterised either by a nucleotide sequence corresponding to the nucleotide sequence substantially as set forth in <400>28 or a nucleotide sequence capable of hybridising to <400>28 under low stringency conditions at 42° C. or a variant, mutant or homologue of said actinomycete.

Preferably, the subject actinomycete corresponds to PM228.

In yet another aspect the present invention provides an isolated actinomycete wherein said actinomycete is characterised either by a nucleotide sequence corresponding to the nucleotide sequence substantially as set forth in <400>29 or a nucleotide sequence capable of hybridising to <400>29 under low stringency conditions at 42° C. or a variant, mutant or homologue of said actinomycete.

Preferably, the subject actinomycete corresponds to PM252.

In still another aspect the present invention provides an isolated actinomycete wherein said actinomycete is characterised either by a nucleotide sequence corresponding to the nucleotide sequence substantially as set forth in <400>30 or a nucleotide sequence capable of hybridising to <400>30 under low stringency conditions at 42° C. or a variant, mutant or homologue of said actinomycete.

Preferably, the subject actinomycete corresponds to PM342.

Reference to the subject actinomycete being “characterised” by the subject nucleotide sequence should be understood to have the same meaning as hereinbefore defined. Similarly, reference herein to “low stringency conditions” has also been previously defined. “Variants”, “mutant” and “homologue”, when defined in terms of actinomycetes, has also been previously defined.

Yet another aspect of the present invention is directed to metabolites derived from the novel actinomycetes hereinbefore defined and derivatives, homologues, analogues, chemical equivalents, mutants and mimetics of said metabolites.

Reference to “metabolite” and “derivatives, homologues, analogues, chemical equivalents, mutants and mimetics” has the same meaning as hereinbefore provided.

Yet another aspect of the present invention is directed to antibodies to the novel actinomycetes or metabolites hereinbefore defined or derivative, homologue, analogue, chemical equivalent, or mimetic of said antibody. Antibodies may be utilised, inter alia, to screen for the subject actinomycetes or to function as an antagonistic agent to the functional activity of the subject actinomycetes. Antibodies may also be directed to metabolites produced by the novel actinomycetes hereinbefore defined. Such antibodies may be monoclonal or polyclonal and may be selected from naturally occurring antibodies or may be specifically raised. In the case of the latter, an antibody may be raised to the actinomycete in its active or attenuated form or it may be raised to an antigen or epitope isolated from said actinomycete. To the extent that an antigen or epitope is utilised, it may first require association with a carrier molecule. Alternatively, fragments of antibodies may be used such as Fab fragments. Furthermore, the present invention extends to recombinant and synthetic antibodies and antibody hybrids. A “synthetic antibody” is considered herein to include fragments and hybrids of antibodies.

The identification of novel actinomycetes of the present invention, and the metabolites derived therefrom, now facilitates the development of both agricultural and medical applications. For example, the novel actinomycetes of the present invention and metabolites derived therefrom are particularly useful, but in no way limited to:
(i) improving plant productivity by, inter alia, providing the subject plant with bio-control capabilities and up-regulating growth promoting activities.
(ii) facilitating biodegradation of non-degraded or only partially degraded organic or inorganic material (herein referred to as “biodegradable material”).
(v) medical/therapeutic applications by the use of metabolites as medicine to treat diseases in humans and animals.
(iv) use of metabolites for agricultural application.
(v) use of actinomycete as an agent for the introduction of genetic material to plants, plant tissues or plant cell culture.

(vi) use of actinomycete as a microbial partner to enhance phytoremediation.

Accordingly, still another aspect of the present invention is directed to the use of the novel actinomycetes hereinbefore defined and metabolites derived therefrom in relation to therapeutic and prophylactic applications in respect of both medical purposes and for nay non-medical purpose sch as agricultural purposes.

The present invention is further defined by the following non-limiting Figures and/or Examples.

### EXAMPLE 1

### Endophytic Actinomycete Isolation

Sampling and Isolation

Plants from 9 fields from three major wheat growing regions in South Australia were sampled at 6-7 week intervals across the growing season. The sites sampled on the Eyre Peninsula were Tuckey, Lock, Yabmana and Yabmana*. Yabmana* was adopted as a sample site at the 11 week sampling when it was observed that the crop in this field was particularly vigorous. These sample sites were characterised by sandy alkaline soils and relatively low rainfall (Tuckey, rainfall=330 mm/year). The sites sampled in the South-East region were Bool Lagoon, Struan and Wolseley. These were characterised by cracking clay soils and higher rainfall. The sites sampled in the mid-North region were Avon and Wild Horse Plains. These were of a loamy earth type soil. Avon was chosen as a sample site as this soil has shown to be suppressive to Rhizoctonia root rot of wheat and Take-all (Ggt) (Roget et. al, 1999). These plants were used for endophyte isolation using protocol

Endophyte Isolation from Root Tissue

Wheat plants were left to air dry for 48 hours before being thoroughly washed to remove all soil from the root mass. The roots were then excised and the shoots discarded. The roots were then subjected to a three-step surface sterilisation procedure. This involved a 60 second wash in 99% ethanol, followed by a 6 minute wash in 3.125% NaOCl, followed by a 30 second wash in 99% ethanol and then a final rinse in sterile RO water. Some of these fragments were then rapidly dipped in 100% ethanol and flamed, then placed onto the plate. These surface-sterilised roots were then aseptically sectioned into 1 cm long fragments and plated onto the isolation media as shown in section 1.4 below, and incubated at 27° C. for 4 weeks.

Endophyte Isolation from Seed

A method for the isolation of endophytes from wheat seeds was developed. After surface sterilising the seeds (with a 60 second wash in 99% ethanol, followed by a 6 minute wash in 3.125% NaOCl, followed by a 30 second wash in 99% ethanol and then a final rinse in sterile RO water) each individual seed was sliced aseptically into 5 slices. These seed slices were placed onto the isolation media, as shown below. The slices were left for 4 weeks at 27 C for any endophytes to appear.

Isolation Media

Several isolation media were used throughout the experiment, the recipes are given below. For each plant, root fragments were plated onto the following isolation media. All plant fragments were plated onto TWYE and HV, YCED, FYSC and FCC. All media were supplemented with BenlateR (active ingredient Benomyl) at 50 mg/l to control fungal contamination.

Tap Water Yeast Extract medium (TWYE) per litre of tap water: Yeast Extract 0.25 g, K₂HPO₄ 0.5 g, Agar 18.0 g. Adjust pH to 7.2.

Humic acid Vitamin B medium (HV) per litre RO water: Humic Acid 1.0 g in 10 ml, 0.2M NaOH, Na₂HPO₄ 0.5 g, KCl 1.71 g, MgSO4.7H₂O 0.05 g FeSO4.7H₂O 0.01 g, CaCO3 0.2 g, Agar 18.0 g, Vitamin B solution (100×) 10.0 ml after autoclave. Adjust pH to 10.0.

Vitamin B solution (100×) per 100 ml RO water: Thyamine hydrochloride 5 mg, Riboflavin 5 mg, Niacin 5 mg, Pyridoxine hydrochloride 5 mg, Inositol 5 mg, Calcium pantothenate 5 mg, Para-amino benzoic acid 25 mg, Biotin 25 mg. Adjust pH to 4.5 and filter sterilise.

Yeast Extract Casamino Acids medium (YCED) per litre RO water: Yeast Extract 0.3 g, Casamino Acids 0.3 g, D-Glucose 0.3 g, K₂HPO₄ 2.0 g, Agar 18.0 g. Adjust pH to 7.2.

Flour Yeast Extract Sucrose Casein Hydrolysate medium (FYSC) per litre RO water: Plain Flour 6 g, Yeast Extract 0.3 g, Casein Hydrolysate 0.3 g, Calcium Carbonate 0.3 g, Sucrose 0.3 g, Agar 18 g. Adjust pH to 7.2.

Flour Calcium Carbonate medium (FCC) per litre of RO water: Plain Flour 4 g, Calcium carbonate 0.4 g, Agar 16 g. Adjust pH to 7.2

All media were autoclaved for 15 min at 121° C.

### EXAMPLE 2

### Actinomycete Characterisation Using Partial 16S rDNA Sequencing

Methodology

DNA Extraction from Actinomycetes

For each isolate to be extracted, a loopful of mycelium and spores were scraped from solid growth media and suspended in 400 ul of saline-EDTA (0.15 M NaCl, 0.1M EDTA pH 8.0) by vortexing. To this, 10 ul of lysozyme was added which was then incubated at 37° C. for 45 minutes. Following this, 10 ul of 1% (w/v) proteinase K and 10 ul of 25% SDS was added followed by incubation at 55° C. for 30 min. A further 10 ul of 25% SDS was added and the tubes were re-incubated at 55° C. for 30 minutes. The tubes were then centrifuged at 10000 rpm for 5 mins in a microcentrifuge to pellet the cell debris, and the supernatant was transferred to a new tube. An equal volume of TE-equilibrated phenol was added to the supernatant, and mixed. The phases were then separated by centrifugation, and the upper aqueous layer transferred to a new tube. The aqueous layer was then extracted with an equal volume of chloroform and mixed, and again the phases separated by centrifugation. Again the upper aqueous layer was transferred to a new tube. To this 2 volumes of ice cold ethanol was added and mixed. The tubes were then held overnight at 4° C., to allow precipitation of the DNA. The precipitate was pelleted by centrifugation (10000 rpm, 5 minutes), and resuspended in 70% ethanol. The suspension was pelleted by centrifugation as above and the supernatant was removed. The pellet was then left to air-dry in the laminar flow hood before being re-suspended in 20 ul of sterile RO water. This solution was purified using a prep-a-gene kit (BioRad) according to the manufacturers instructions, with 10 ul of DNA binding matrix. This kit is used to purify, concentrate and desalt the DNA to make it suitable for PCR. This kit involves the use of a DNA binding matrix. The matrix, with bound DNA is then washed using ethanol and iso-propyl alcohol to remove contaminants. The DNA is then eluted from the matrix with water. The extracts were stored at −20° C.

Partial 16S rDNA Sequencing

DNA for 16S rDNA sequencing was prepared for each actinomycete. The primers for this PCR reaction were “universal” 16S rDNA primers and should amplify the 16S gene for any bacteria. The primers are designed to amplify a region between position 27 and 765 of the 16S rRNA gene (based on E. coli base numbering) in actinomycetes, and should yield a PCR product of approximately 738 bp long. The primers are designated 27f (5′ AGAGTTTGATCMTGGCTCAG) (<400>31) (where M is adenine or cytosine) and 765r (CTGTTTGCTCCCCACGCTTTC) (<400>32). PCR reactions were done in 100 ul reaction volumes with the following reagents: 27f (200 ng.ul⁻¹) 4 ul, 765r (200 ng.ul⁻¹) 4 ul, 5× taq buffer (5% 40 mM dNTP's, 40% 25 mM MgCl, 50% 10× PCR buffer, 5% water) 20 ul, water 67 ul, Taq polymerase (2 U.ul⁻¹) 1 ul, template DNA 4 ul. The reactions were run using the following profile: 94° C.-8 mins, (94° C.-1 min, 45° C.-1 min, 72° C.-2 min)×30 cycles, 45° C.-1 min, 72° C.-10 min.

The PCR products were purified using the protocol “Preparation of PCR products for sequencing”, detailed herein. The products were sequenced using a Hewlett Packard automated sequencer and the 765r primer. The products were sequenced undiluted from the Wizard kit and the primer was supplied at 22 ng.ul⁻(3.2 picomole.ul⁻). The sequences obtained were compared to online databases using BLAST (Altschul et al., 1997) on the National Centre for Biotechnology Information (NCBI) website (www.ncbi.nlm.nih.gov). The standard blastn (nucleotide-nucelotide) algorithm was used with the default settings. The three highest match coefficients by the bit score were entered into the table.

Full rDNA Sequencing of those Endophytic Actinomycetes with Bio-Control or Growth Promotion Activity

The isolates that showed activity in the growth promotion and/or bio-control assays were chosen for detailed characterisation using full 16S rDNA sequencing.

DNA for 16S sequencing was prepared for the selected isolates using the protocol “DNA isolation and purification from actinomycetes”, detailed hereinbefore. The 27 to 1492 region of the 16S gene was amplified using the 27f (5′ AGAGTTTGATCMTGGCTCAG) (<400>31) (where M is adenine or cytosine) and the 1492r (5′ TACGGYTACCTTGTTACGACTT) (<400>33) (where Y is cytosine or thymine) primers. The reaction profile was identical for the amplification of the 27-765 region of the 16S gene. The resultant 1465 bp PCR product was purified using the protocol “Preparation of PCR products for sequencing” detailed hereinbefore, and sent for sequencing.

To allow sequencing of the complete PCR product, four sequencing primers were needed, as automated sequencing reads only up to 500 bp of sequence. These primers, and their sequences are given below.

| | |
|---|---|
| Primer label | Sequence |

| | |
|---|---|
| 27f | 5′ AGAGTTTGATCMTGGCTCAG <400>32 |
| 765r | 5′ CTGTTTGCTCCCCACGCTTTC <400>33 |
| 704f | 5′ GTAGCGGTGAAATGCGTAGA <400>35 |
| 1492r | 5′ TACGGYTACCTTGTTACGACTT <400>34 |

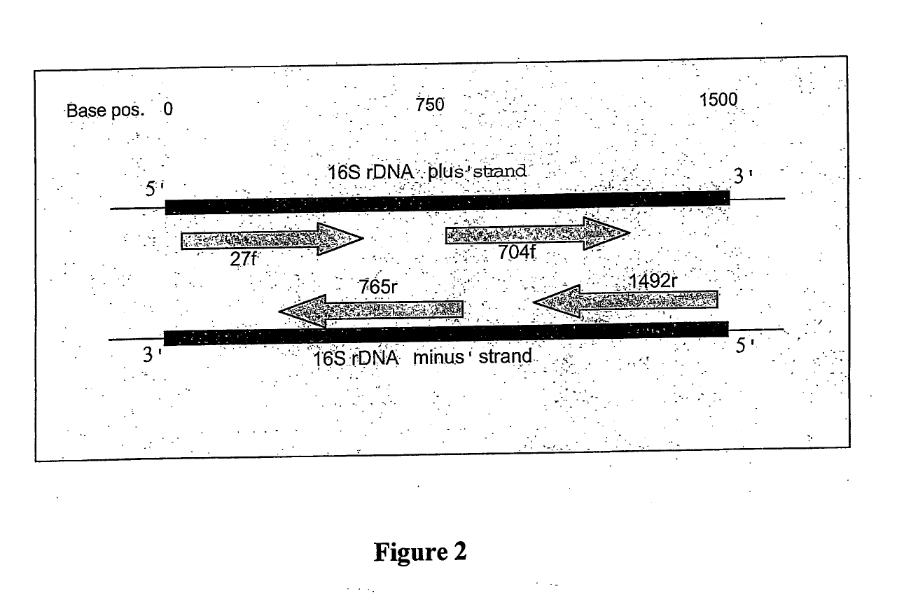

The 27f primer is used to read from position 27 to 500 of the gene, 765r reads from 765 back to approximately base 200, the 704f reads from position 704 to approximately 1200, while the 1492r primer reads from 1492 back to approximately position 1000 of the gene. This is summarised in FIG. 2.

Due to the reverse primers reading from the ‘minus’ strand of the 16S rDNA gene, these sequences had to be reverse complemented, ie. read from back to front and as a complement, to make them read in the same strand and orientation as the forward primers. Once this was done, the four fragments were then aligned using the parts of the sequence that overlap between each fragment to assemble the complete sequence from position 27 to 1492 (based on E. coli numbering). The final complete sequence was then submitted to a BLAST search and the matches recorded.

Agarose Gel Electrophoresis

All DNA samples were analysed using agarose gel electrophoresis. 2% agarose in 0.5× TBE were used in all cases. Gels were run in 0.5× TBE buffer at 90V. DNA samples were prepared for electrophoresis by mixing 5 ul of sample with 1 ul of agarose gel loading buffer. Samples were then loaded with the gel submerged in ½ TBE in the gel tank. Once run, gels were stained for 20-30 minutes in ethidium bromide, then destained in water for 15-30 minutes. DNA in the gels was visualised using a UV transilluminator and a Tractel image capture system.

Preparation of PCR Products for Sequencing

PCR products were prepared for sequencing using a Promega Wizard PCR prep kit. The manufacturer-supplied protocols ‘Direct purification of PCR amplifications’ and ‘Purification using a vacuum manifold’ were followed before sending the samples to an automated sequencing facility. As with the prep-a-gene kit described in “DNA extraction and purification from actinomycetes”, this kit also uses a DNA binding resin, which preferentially binds DNA fragments in the 200-2000 bp range. The PCR reaction reagents, the genomic DNA and the primers are then washed away using iso-propyl alcohol. The DNA is then eluted from the DNA-binding resin using water.

Results

Sequencing Results

Table 2 shows the full 16S rDNA sequence matches of selected isolates with bio-control or growth promotion activity. The bit score indicates both the length and accuracy of the sequence match, while the percentage describes the accuracy of the match.

Table 3 shows the actinomycete identification summary of a second batch of actinomycete isolates.

### EXAMPLE 3

### Streptomyces triticum

Description of Streptomyces tritictum gen nov. sp. nov

This description relates members of the major group consisting of endophytic actinobacterial strains EN19, EN23, EN 27, EN 28, EN 35, EN57, SE1, SE 2, PM36, PM40, PM41, PM 87, PM110, PM119, PM171, PM228 and PM252.

Streptomyces triticum gen nov. sp. nov. is a saprophytic actinobacteria, with endophytic capabilities. These members of this genus were isolated from surface-sterilised tissue of healthy wheat and barley plants. The healthy cereal samples were collected from 12 different districts within the South Australian cereal belt.

The spores are smooth and cylindrical and attached end to end with a diameter of 0.4 um and length of 0.8-1.4 um. The colony characteristics on the International Streptomyces Project media include production of white to cream spores and a dark soluble pigment on International Streptomyces Project Medium (ISP) 2. On ISP 3, the spores were white in colour, and the strains have variable pigmentation, which was either colourless, cream or brown. On ISP 4 the strains belonging to this new species produced spores that were mostly white in colour with either no soluble pigment or a light brown colour. On ISP 5 and ISP 7 the isolates showed the presence of melanin production with spore colour varying between brown to black or white. Nutrient agar showed no pigment formation and all isolates had white spores.

All strains belonging to this species showed glucose assimilation. The isolates did not utilise inositol, cellulose, dulcitol, rhamnose or arabinose. Of the other carbohydrate tested, the isolates showed variable utilization. All isolates showed hydrolysis of starch, gelatin and urea. The isolates showed variable H₂S production, nitrate reduction, and peptonisation and coagulation of milk. All isolates were inhibited by concentrations of streptomycin ranging from 1-1.5 μg/ml. All isolates had a minimum pH tolerance of between pH 3-4, and a maximum salt tolerance of between 6-8%. The optimum temperature for growth was 27° C. with no growth seen at 45° C. for any of the strains belonging to Streptomyces triticum.

Description of Streptomyces trificum var. griseoviride gen nov. sp. nov

Thid description relates to the minor variants which are melanin-negative consisting of strains EN5, EN 16, EN 17, PM 144, PM 185, PM 208 and PM 342.

Streptomyces triticum var. griseoviride gen nov. sp. nov. is a saprophytic actinobacteria, with endophytic capabilities. These strains were isolated from surface-sterilised tissue of healthy wheat and barley plants. The healthy cereal samples were collected from 12 different districts within the South Australian cereal belt.

The spores are smooth and cylindrical and attached end to end with a diameter of 0.4 um and length of 0.8-1.4 um. The colony characteristics on the International Streptomyces Project media include production of white/cream, grey or green spores and no soluble pigment on International Streptomyces Project Medium (ISP) 2. On ISP 3, the spores were white or green in colour, and the isolates had no soluble pigmentation. On ISP 4 the strains belonging to this new species produced spores that were white or grey in colour with either no soluble pigment or a light brown colour. On ISP 5 and ISP 7 some of the isolates showed the presence of a light brown soluble pigment with spore colour varying between white or grey or green. Nutrient agar showed no pigment formation and all isolates had white spores.

All strains belonging to this species showed glucose assimilation. The isolates did not utilise cellulose, dulcitol, rhamnose or arabinose. One isolate showed utilisation of inositol and raffinose. Of the other carbohydrate tested, the isolates showed variable utilization. All isolates showed hydrolysis of starch and gelatin. The majority of the isolates showed no H₂S production or peptonisation of milk, but displayed nitrate reduction, and a variable utilization of urea and coagulation of milk. All isolates were inhibited by a 1 μg/ml concentration of streptomycin, and had a minimum pH tolerance of between pH 5, and a maximum salt tolerance of between 6-9%. The optimum temperature for growth was 27° C. with no growth seen at 45° C. for any of the isolates belonging to this variant of Streptomyces triticum.

Comparison with the Nearest Matching Type Strains

The members of the new genus Streptomyces triticum, including variants, showed significant differences with the 2 type cultures that showed the closest match on the basis of their 16S rDNA gene sequences; These were Streptomyces caviscabies (ATCC 51928), Streptomyces setonii (ATCC 25497). Differences between the type cultures and the isolates that belong to Streptomyces triticum gen. nov. sp. nov. are stated below.
In comparison to both type strains the members of Streptomyces triticum gen. nov. sp. nov. showed less than a 98% similarity in the sequence of their 16S rDNA genes, indicating that they belong to a new species. For example, the percentage similarity either of the full gene sequence (denoted with a *) or the partial gene sequence is, for EN 27* (94%), EN 28* (96%), EN 35* (97%), SE 1* (95%), SE 2* (97%), PM 40 (93%), PM 41 (96%), PM 36 (95%), PM 87 (94%), PM 171(93%), PM 228 (96%), PM 252 (92%). For the strains belonging to the variant Streptomyces triticum var. griseoviride gen. nov. sp. nov. the percentage similarity is, for EN16* (95%), EN 17 (90%), PM 144 (97%), PM 185 (98%), PM 208 (95%) and PM 342 (96%)
Streptomyces caviscabies (ATCC 51928) did not show any variation in its spore colour on the various ISP media, whereas the majority of the endophytic isolates belonging to Streptomyces triticum or Streptomyces triticum var. griseoviride had different spore colour on some of the media. Also, there was no significant soluble pigment produced by Streptomyces caviscabies (ATCC 51928). In contrast to this type culture the majority of the endophytic isolates belonging to Streptomyces triticum displayed some pigment production, and all displayed melanin production. This indicates a significant difference between the endophytic isolates and the type culture. The melanin-negative isolates of the variant Streptomyces triticum var. griseoviride differed in the morphological characteristics seen on ISP 2, 4 and 7 that were observed with the Streptomyces caviscabies (ATCC 51928).
Streptomyces setonii (ATCC 25497) had similar morphological characteristics and carbohydrate utilization properties as Streptomyces caviscabies (ATCC51928). There were some differences in biochemical properties, but noe ogf these were identical to strains belonging to Streptomyces triticum or its variant.

Streptomyces caviscabies (ATCC 51928) and Streptomyces setonii (ATCC 25497) type cultures had similar carbohydrate utilization patterns. When compared to the type cultures of Streptomyces caviscabies (ATCC 51928) and Streptomyces setonii (ATCC 25497), the Streptomyces triticum strains and variant strains had significant differences in their carbohydrate utilization from these two type cultures.

Table 4 shows the characterisation of isolates belonging to Streptomyces triticum gen nov. sp.nov.—spore coloration on International Streptomyces Project media, and carbohydrate utilization

Table 5 shows the characterisation of isolates belonging to Streptomyces triticum gen nov. sp.nov.—soluble pigmentation and biochemical analysis.

Table 6 shows the characterisation of isolates belonging to Streptomyces triticum var. griseoviride gen nov. sp.nov.—spore coloration on International Streptomyces Project media, and carbohydrate utilization and comparison with nearest matching type.

Table 7 shows the characterisation of isolates belonging to Streptomyces triticum var. griseoviride gen nov. sp.nov.—soluble pigmentation and biochemical analysis and comparison with nearest matching type.

### EXAMPLE 4

### Application of Endophytic Actinobacteria for the Control of Cereal Diseases (Glasshouse Trials)

Further work investigated the application of endophytic actinobacteria for the control of other cereal diseases, such as Pythium damping-off, in glasshouse trials.

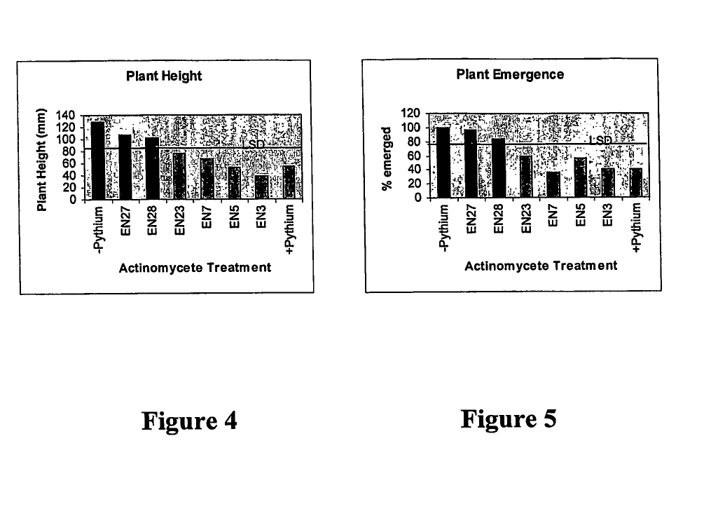

These trials yielded significant results with several actinobacterial endophyte strains giving almost complete symptom control on wheat. It is encouraging to discover that the most effective strains for the control of Pythium were in general the same strains belonging to the Str. triticum species that were effective for the control of Take-all. Therefore, the commercial development of at least one of these strains may have application as a broad-spectrum biofungicide for the control of several diseases. To validate this finding, the trials were repeated a further 3 times, yielding similar results (FIGS. 4 and 5).

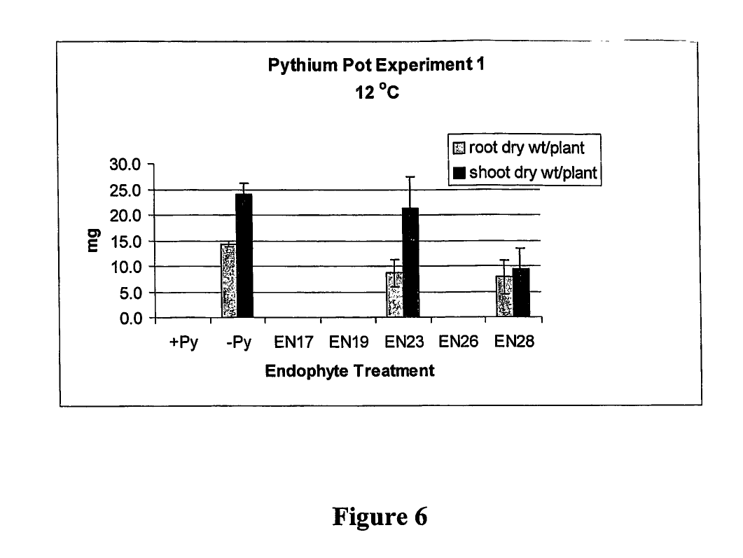

Wheat seeds inoculated with actinobacterial spores and an uninoculated control (+Py) were grown in soil infested with Pythium irregulare. A control treatment with no disease or actinobacteria inoculation was also included (−Py) (FIG. 6).

In the absence of actinobacteria inoculant (+Py) and for 3 of the actinobacteria inoculated treatments (EN17, EN19, EN26), no plants emerged during the duration of the experiment (6 weeks). Where no disease was present (−Py) or seed was inoculated with spores of 2 actinobacterial strains (EN 23, EN28) the wheat seeds were able to emerge and grow.

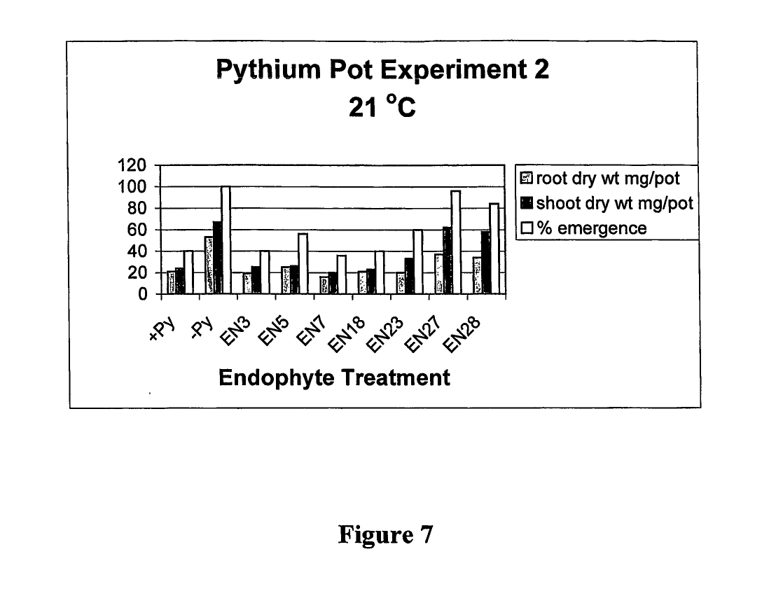

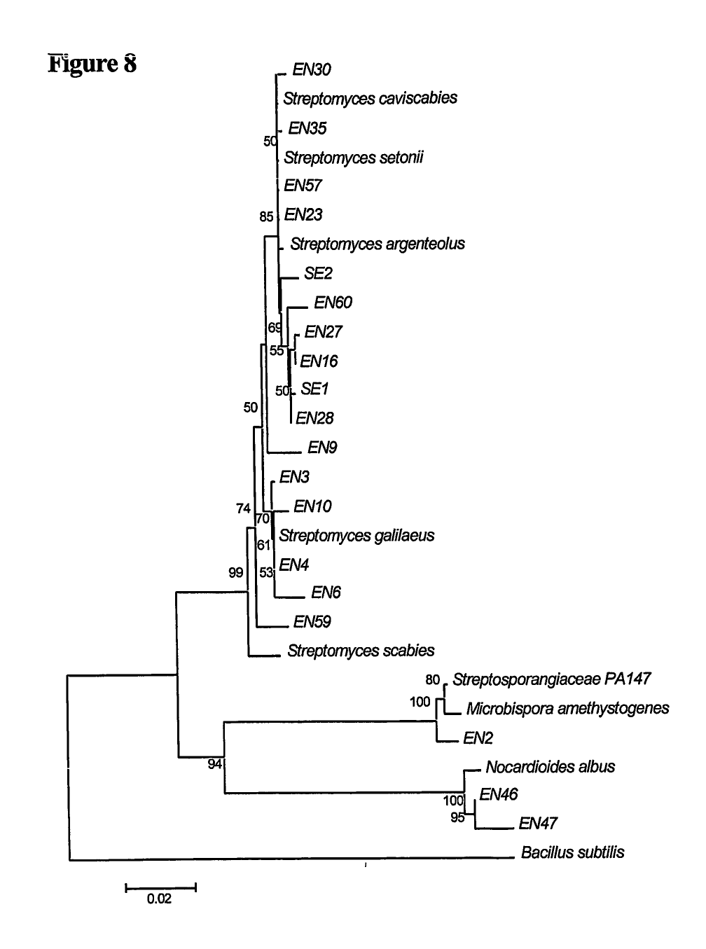

In a second experiment the plants were grown at 21° C. instead of 12° C. At this temperature, the effects of the disease weren't as drastic, however, there were highly significant effects on emergence and growth of the wheat. Root and shoot growth and plant emergence for wheat inoculated with the actinobacterias EN27 and EN28 were significantly higher (P, 0.05) than all other treatments except the treatment where no Pythium was added to the soil (−Py) (FIG. 7).

### EXAMPLE 5

### In-Vitro Inhibition of Gaeumannomyces graminis VAR. Tritici by Endophytic Actinomycetes

Methodology

In-Vitro Antifungal Metabolite Production Assays

This assay was based on the protocol of Crawford et al. (1993). The actinomycetes were streaked into one third of a corn-meal agar (CMA) plate and allowed to grow for 8 days. This time allowed the actinomycete grow, sporulate and to produce secondary metabolites. After 8 days a 5 mm×5 mm block of CMA agar with the fungal pathogen of interest growing on it was introduced to the actinomycete plate. Secondly, blocks of the fungus were also added to at least 3 CMA plates that were not inoculated with the actinomycete. This was done to provide a control measurement of the fungal growth. The mean measure of the radial growth of the fungus on the control plates was compared with the growth of the fungus towards the actinomycete on the test plates to give an indicator of actinomycete antagonism of the fungal pathogen. The control plates were used instead of a measure of fungal growth away from the actinomycete on the test plates as fungal growth was often inhibited in this direction as well, hence artificially reducing the antagonistic effect.

Results

In-Vitro Antifungal Assay

Table 11 below shows the in-vitro antagonism of Gaeumannomyces graminis var. tritici (Ggt) by each of the actinomycete isolates. The strength of the antifungal activity was calculated as a ratio of the growth of the fungus (in mm) on the actinomycete free control plate divided by the growth of the fungus (in mm) towards the actinomycete streak on the test plates.

The results show that 31.5% (18 isolates) of the isolates were able to strongly inhibit (++ or better) at least 1 of the strain of Ggt used in the assay and that 55% of these isolates (10 isolates) were able to strongly inhibit all three strains of Ggt used in the assay.

### EXAMPLE 6

### In-Vitro Inhibition of Rhizoctonia solani by Endophytic Actinomycetes

Methodology

In-Vitro Antifungal Metabolite Production Assays

This assay was based on the protocol of Crawford et al. (1993). The actinomycetes were streaked into one third of a corn-meal agar (CMA) plate and allowed to grow for 8 days. This time allowed the actinomycete grow, sporulate and to produce secondary metabolites. After 8 days a 5 mm×5 mm block of CMA agar with the fungal pathogen of interest growing on it was introduced to the actinomycete plate. Secondly, blocks of the fungus were also added to at least 3 CMA plates that were not inoculated with the actinomycete. This was done to provide a control measurement of the fungal growth. The mean measure of the radial growth of the fungus on the control plates was compared with the growth of the fungus towards the actinomycete on the test plates to give an indicator of actinomycete antagonism of the fungal pathogen. The control plates were used instead of a measure of fungal growth away from the actinomycete on the test plates as fungal growth was often inhibited in this direction as well, hence artificially reducing the antagonistic effect.

Results

In-Vitro Antifungal Assay

Table 12 below shows the in-vitro antagonism of Rhizoctonia solani by each of the actinomycete isolates. The strength of the antifungal activity was calculated as a ratio of the growth of the fungus (in mm) on the actinomycete free control plate divided by the growth of the fungus (in mm) towards the actinomycete streak on the test plates.

The results show that 49.1% (28 isolates) of the isolates were able to strongly inhibit (++ or better) R. solani.

### EXAMPLE 7

### In-Vitro Inhibition of Pythium spp. by Endophytic Actinomycetes

Methodology

In-Vitro Antifungal Metabolite Production Assays

This assay was based on the protocol of Crawford et al. (1993). The actinomycetes were streaked into one third of a corn-meal agar (CMA) plate and allowed to grow for 8 days. This time allowed the actinomycete grow, sporulate and to produce secondary metabolites. After 8 days a 5 mm×5 mm block of CMA agar with the fungal pathogen of interest growing on it was introduced to the actinomycete plate. Secondly, blocks of the fungus were also added to at least 3 CMA plates that were not inoculated with the actinomycete. This was done to provide a control measurement of the fungal growth. The mean measure of the radial growth of the fungus on the control plates was compared with the growth of the fungus towards the actinomycete on the test plates to give an indicator of actinomycete antagonism of the fungal pathogen. The control plates were used instead of a measure of fungal growth away from the actinomycete on the test plates as fungal growth was often inhibited in this direction as well, hence artificially reducing the antagonistic effect.

Results

In-Vitro Antifungal Assay

Table 13 below shows the in-vitro antagonism of Pythium spp. by each of the actinomycete isolates. The strength of the antifungal activity was calculated as a ratio of the growth of the fungus (in mm) on the actinomycete free control plate divided by the growth of the fungus (in mm) towards the actinomycete streak on the test plates.

The results show that 22.8% (13 isolates) of the isolates were able to strongly inhibit (++ or better) 1 of the strains of Pythium used in the assay and that 46% of these isolates (6 isolates) were able to strongly inhibit both strains of Pythium used in the assay.

### EXAMPLE 8

### In Vitro Inhibition of Fusarium Graminearum by Endophytic Actinomycetes

Methodology

In-Vitro Antifungal Metabolite Production Assays

This assay was based on the protocol of Crawford et al. (1993). The actinomycetes were streaked into one third of a corn-meal agar (CMA) plate and allowed to grow for 8 days. This time allowed the actinomycete grow, sporulate and to produce secondary metabolites. After 8 days a 5 mm×5 mm block of CMA agar with the fungal pathogen of interest growing on it was introduced to the actinomycete plate. Secondly, blocks of the fungus were also added to at least 3 CMA plates that were not inoculated with the actinomycete. This was done to provide a control measurement of the fungal growth. The mean measure of the radial growth of the fungus on the control plates was compared with the growth of the fungus towards the actinomycete on the test plates to give an indicator of actinomycete antagonism of the fungal pathogen. The control plates were used instead of a measure of fungal growth away from the actinomycete on the test plates as fungal growth was often inhibited in this direction as well, hence artificially reducing the antagonistic effect.

Results

In-Vitro Antifungal Assay

Table 14 shows the in-vitro antifungal activity of the selected actinomycete endophytes against Fusarium graminearum actinomycetes.

### EXAMPLE 9

### In-Planta Wheat Root Growth and Germination Regulation of Endophytic Actinomycetes

Methodology

In-Planta Early Growth Promotion Assays

Each of the isolates was tested using 25 individual plants. These were arranged as 5 plants in each of 5 pots which were rotated in the glasshouse at regular intervals to remove any positional effects of the pots. This layout of plants allowed for analysis of variance and t-tests to be performed on the results.

Seeds were surface sterilised using a 6 minute wash in 3.125% sodium hypochlorite, followed by washing in sterile water. Following surface sterilisation, batches of approximately 50 seeds were made and coated with a spore suspension of each isolate made from one 9 cm petri dish of well grown actinomycete culture mycelium and spores, in 3 ml of sterile RO water. These suspensions were then poured over the seeds and allowed to dry overnight in a laminar flow cabinet. Control batches were produced by soaking surface sterilised seeds in 3 ml of sterile water and left to dry overnight in a similar manner.

Endophytes were tested for their ability to enhance root or shoot growth of young wheat. The trial was broken up into batches of 10 treatments, each with a set of untreated controls, Pot trials were set up as above and seeds coated using the above protocol. The wheat seeds were planted at a depth of approximately 2 cm into steamed recycled UC soil. The plants were then left to grow for 4 weeks, at which point they were harvested. The plants were then washed and allowed to dry for 2 weeks until brittle. Root and shoot dry masses were taken of all individual plants to give a mean for each treatment and for the control for the batch. Percentage increases or decreases for each treatment relative to the control were calculated to give a standard score that could be used to compare plants between batches. Following this the mean for each treatment was compared to the control and the statistical significance of the differences observed was calculated using a paired t-test.

Results

In-Planta Early Growth Promotion Assays

Table 15 shows the growth regulatory effects of each of the endophytes tested in the early growth promotion assays, while table 16 shows a summary of the results indicating those isolates that show significant (p<0.05 or p<0.10) growth regulatory effects.

### EXAMPLE 10

### In-Planta Gaeumannomyces graminis var. tritici biocontol in Steamed Soil by Endophytic Actinomycetes

Methodology

In-planta steamed soil Gaeumannomyces graminis var. tritici (Ggt) bioassay

For each endophyte tested, 5 pots each planted with five endophyte-coated seeds were used. These pots were randomly spread out in the glasshouse, and rotated routinely to remove any positional effects of the pots. The control was also planted in the same way, except the seeds were coated with water containing no actinomycete spores or mycelium. Gaeumannomyces graminis var. tritici 8 (Ggt8) inoculum was added to steamed UC soil mix at a rate of 180 propagules/kg. Pots were half filled with this infested soil, then a 2 cm layer of uninfected soil was layered over this. Onto this layer the seeds were planted and then they were overlayed with a further 2 cm layer of uninfected soil.

The plants were allowed to grow for 4 weeks and then scored for Ggt disease symptoms. Scoring involved examining the seminal roots of the plant (ie those roots that emerge directly from the seed) and calculating a percentage on how many of these roots (there are five) exhibit the black lesions along the root characteristic of Ggt infection. A mean level of infection was calculated for each endophyte treatment and for the control. Each endophyte treatment was compared to the control and the statistical significance of any observed differences was assessed using 2-tailed t-tests.

Results

Steamed Soil Ggt Bio-Control Assays

Table 17 shows the in-planta Ggt bio-control activity of the actinomycete endophytes, those isolates listed in bold are those closely related to Streptomyces caviscabies. The numbers in blue show a result with a t-test p-value of less than 0.10, while those in red indicate a t-test p-value of less than 0.05.

Of the 10 S. triticum-like isolates, 8 of them showed Ggt disease reductions of greater than 25% and 7 of these 8 had T-test p-values of less than 0.055. This would suggest that the group of S. triticum-like isolates had a high incidence of Ggt bio-control activity.

### EXAMPLE 11

### In-Planta Gaeumannomyces graminis var. tritici and Rhizoctonia solani Bio-Control in Field Soil by Endophytic Actinomycetes

Methodology

In-Planta Gaeumannomyces graminis var. triici (Ggt) Bio-Control Infield Soil

For those isolates that showed activity in the first Gaeumannomyces graminis var. tritici (Ggt) bio-control assay, in the steamed soil with artificial inoculum, a second assay was performed using a field soil naturally infected with both Ggt and Rhizoctonia solani. This soil was taken from a paddock on the “Rolling Hills” property in Peake, SA. This assay was performed as detailed earlier except that no extra Ggt inoculum was added to the soil and no layers of clean soil were introduced into the pot. As above the plants were allowed to grow for 4 weeks. After this period the plants were harvested and the seminal roots were scored for Ggt infection (as described above) and Rhizoctonia solani infection (which is characterised by roots with broken off tips which are blackened and form a point—“spear tips”).

Results

Field Soil Ggt Bio-Control

Table 18 shows the field soil Ggt bio-control activity of the isolates. The percentage disease reductions reported are all relative to a batch of control plants that were planted into the infected soil with no actinomycete seed coating.

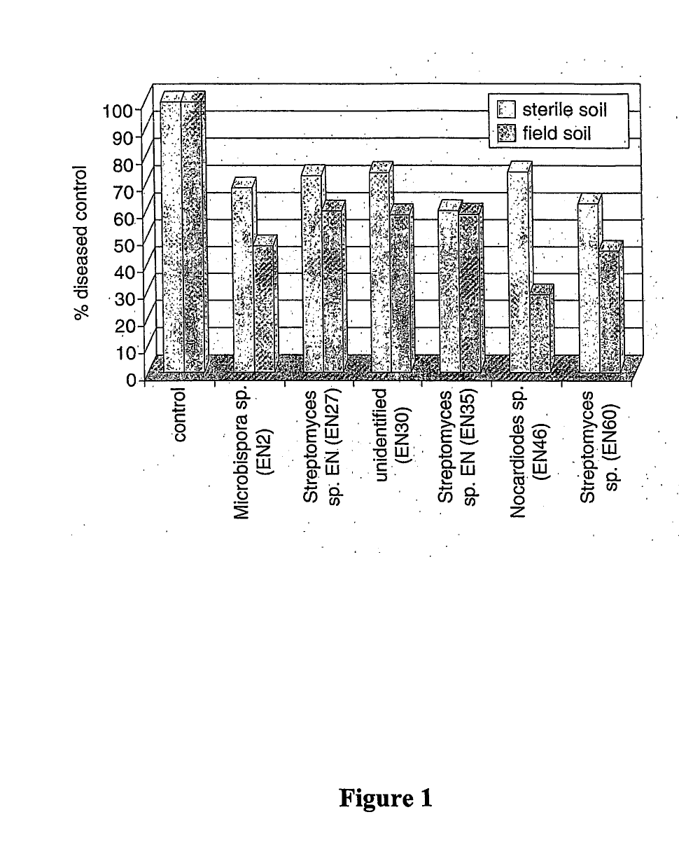

The field soil used for this assay was naturally infected with both Ggt and Rhizoctonia solani, and disease ratings were taken for both pathogens. FIG. 1 shows a graphical representation of disease control for those isolates with statistically significant results. The results of the field soil assay were markedly more variable than those in the sterile soil, meaning quite large disease reductions were not significantly significant (eg. Some isolates had disease reductions of >30% but p>0.05). Several of the isolates such as EN2, EN9, EN22, EN23, EN43, EN57 and EN60 also exhibited control of Rhizoctonia, indicating these isolates may have broad spectrum antifungal activity.

For those isolates with significant activity in the field soil it was observed that the magnitude of the bio-control activity, ie the disease reduction was generally greater in the field soil, than was observed in the steamed soil assay.

### EXAMPLE 12

### In-Planta Aphid Bio-Control

Long-Term Growth Promotion Assays—Aphid Resistance

Selected isolates EN3, EN6, EN10, EN16, EN27, EN28, EN57, EN59, SE1 and SE2 were tested for their ability to induce resistance to foliar pests, i.e, in an aphid challenge assay. These assays were set up as in the early growth promotion assays with some modifications to the protocol. Five seeds were planted into steamed UC soil mix in larger (150 mm diameter) pots. The plants were then left to germinate. Once the plants had emerged, each pot was weeded to have only 3 plants per pot to eliminate compensatory growth effects in those pots with fewer plants.

The plants were then exposed to aphids 10 weeks after germination, and scored for infestation.

Aphid resistance observed

During the aphid infestation, it was observed that some pots were more heavily infested with aphids than others. Some observations were made on this attack.

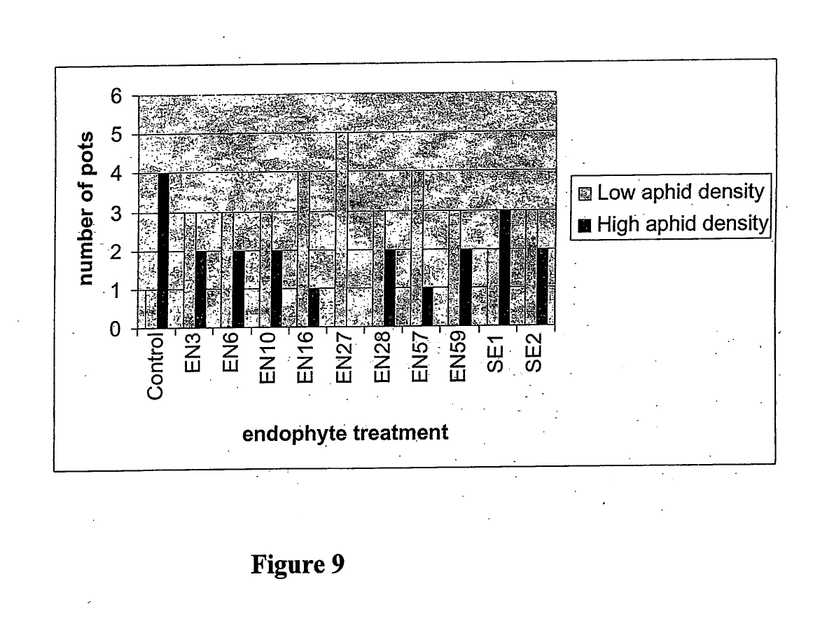

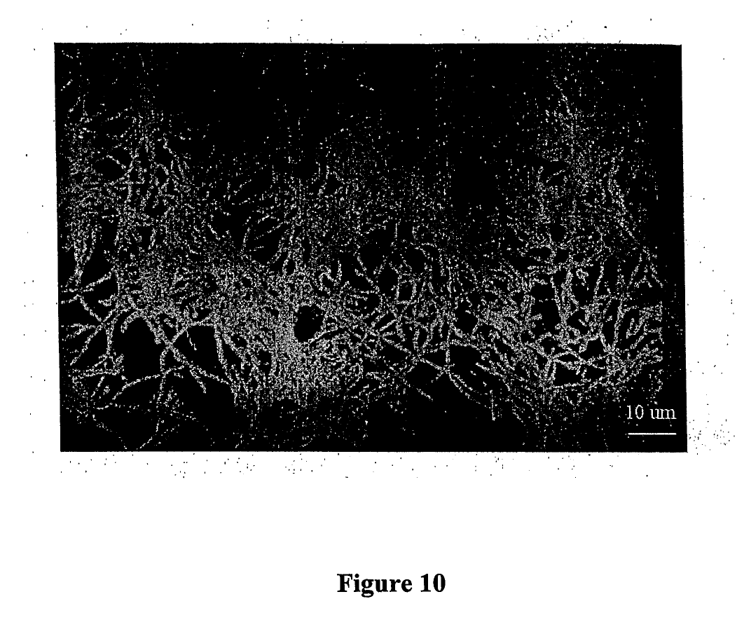

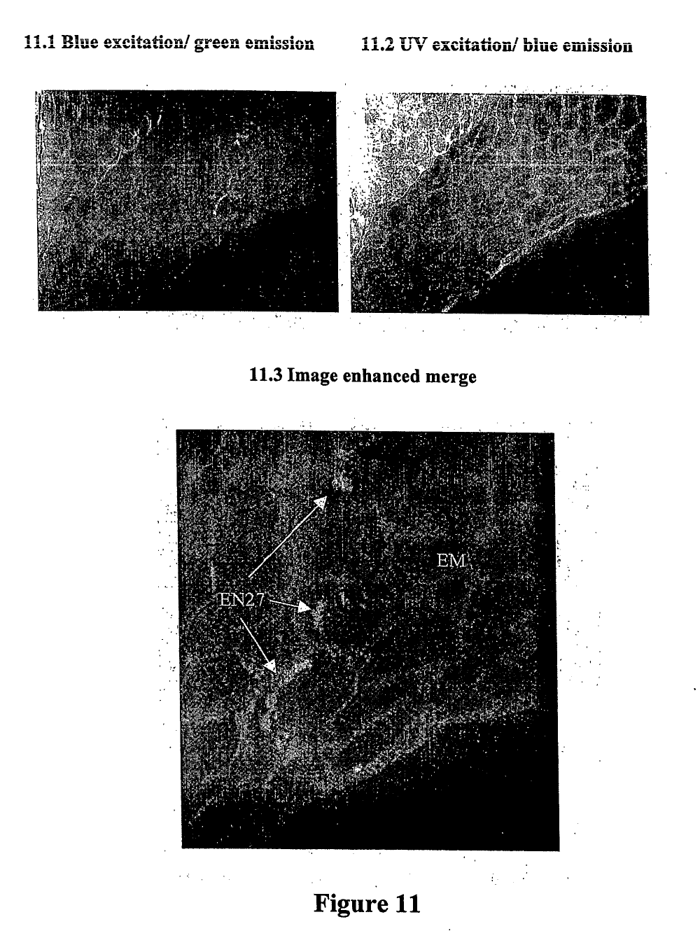

The pots were blindly classified into those pots with high levels of aphid infestation of the plants, and those pots with low levels of aphid infestation of the plants. The number of pots for each endophyte treatment, at each of the aphid infestation levels was counted. The results are shown in FIG. 9.

### EXAMPLE 13

### Growth and Germination of Barley and Oats

Table 19 shows the effect of inoculation with actinomycete endophyte on the growth and germination of barley and oats in comparison to untreated control plants. This was carried out in 5 replicate pots containing 3 plants each.

Table 20 shows the effect of inoculation with actinomycete endophytes from the second batch, on the growth and germination of wheat plants in comparison to untreated control plants. This was carried out in 5 replicate pots containing 3 plants each. The results shown below are all p<0.05.

### EXAMPLE 14

### Visualisation of an Endophytic Streptomyces sp. in Wheat Seed Using Green Fluorescent Protein

Materials and Methods

Construction of the egfp-Tagged Streptomyces sp. EN27.

Transformation of Streptomyces sp. EN27 with EGFP was performed as set out in Coombs and Franco (Appl. Environ. Microbiol. 69(7):4260-4262, 2003).

The plasmid DNA of the E. coli DH5α was extracted using a Wizard Plus SV miniprep kit (Promega). This plasmid DNA was then used to transform competent E. coli S17.1, which could be used for intergeneric recombination with Streptomyces, as it carries an integrated form of RK4 transfer genes necessary to integenerically transfer plasmids that carry the oriT/RK2 regions (Flett, F. et al. 1997; Mazodier, P. et al. 1989).

The intergeneric recombination protocol used was that of Flett et al. (1997) which is a modification of the method of Mazodier et al. (1989) as described in Practical Streptomyces Genetics gieser, T. et al. 2000). Expression of egfp was detected using epifluorescence microscopy.

Inoculation of Wheat cv. Excalibur with Streptomyces sp. EN27pIJ8641.

Approximately 100 seeds were prepared for coating by surface sterilisation using a six minute wash in 3.125% NaOCl, followed by three double volume rinses in sterile RO water. After the final water rinse was drained off, the seeds were evenly separated into two petri dishes. The egfp-expressing actinomycete, Streptomyces sp. EN27 pIJ8641, was inoculated onto TSA plates supplemented with apramycin at 50 ug.ml⁻¹. These plates were incubated at 27° C. until the cultures had sporulated. The mycelium was then scraped off the plate and transferred to a sterile eppendorf tube. 1.5 ml of sterile RO water was added to the tube, which was then vortexed thoroughly to ensure even distribution of the mycelium and spores in suspension. This spore suspension was added to one of the batches of seed, while 1.5 ml of sterile RO water was added to the other batch of seed, to act as a control. The seeds were then dried overnight in a laminar flow cabinet. Some of this seed was then placed on a mannitol-soy flour (MS) medium plate and the seeds were allowed to germinate. 5 inoculated seed were planted aseptically, in duplicate, into autoclaved sterile sandy-loam soil placed in sterile 500 ml screw-capped flasks to a depth of 7 cm. The flasks were watered with sterile water and the lids loosely screwed on, and incubated in a plant growth chamber with a 16 hour light-8 hour dark cycle at 25° C.

Visualisation of egfp-Expressing Pure Cultures of Streptomyces sp. EN27 using LSCM.

The cultures were grown on MS medium without selective pressure in triplicate, until thick growth had occurred. Loopfuls were taken off each of these plates and smeared onto microscope slides. A drop of sterile water was placed on the smear, before being covered with a glass coverslip, which was then sealed with nail polish.

The slides were visualised using a Nikon laser scanning confocal microscope with a Krypton/Argon laser with a 488 nm emission filter, at 30% power with a green light detection filter. A range of other filter sets and higher laser power was tested to ensure that the fluorescence observed was due to the egfp gene product, and not to the autofluorescence of the actinomycete. Control strains, ie. non-transformed strains of Streptomyces sp. EN27, were also examined to ensure the non-transformed actinomycetes had no autofluorescence in the green range.

Visualisation of egfp-Expressing Streptomyces sp. EN27 in seed Sections using Epifluorescence Microscopy.

Seeds coated with Streptomyces sp. EN27 and untreated control seeds were cut into 60-80 um sections using a Leitz Wetzlar microtome with a freezing stage attachment after 24 hour incubation on MS agar medium. These sections were placed onto microscope slides and mounted in water under a glass coverslip, which was then sealed using nail polish.

Prepared slides were examined under an Olympus BX-50 microscope using a mercury vapour lamp. The filter block used to visualise the EGFP-expressing actinomycetes was a Chroma 31001 with an excitation filter of 465-495 nm and an emission filter of 515-555 nm. The structure of the plant tissue was visualised using the autofluorescence of the tissue. Several filter sets were tried, and the best visualisation was found to be with UV excitation and blue emission. These wavelengths were obtained using an Olympus U-MNUA filter set. This filter set gave an excitation wavelength of 360-370 nm and an emission wavelength of 420-460 nm. These filters were the most appropriate as the EGFP molecule has very little excitation or blue light emission under UV light, and the Chroma 31001 filter block encompasses the peak excitation and emission wavelengths of the EGFP molecule (488 nm excitation and 520 nm emission).

This procedure was repeated on a daily basis for seeds that had been incubated for a further 3 days and undergone germination.

Results

Transformation of E. coli S17.1 with pIJ8641.

700 ng of plasmid DNA was used for the transformation of E. coli S17.1.400 transformants were recovered to give a transformation efficiency of 5.7×10² transformants per ug of plasmid DNA. No transformants were seen on the control reaction plate.

Intergeneric transformation of Streptomyces sp. EN27 with E. coli S17.1 pIJ8641.

After 16 hours incubation, a thin mat growth was observed on the transformation plates. To this was added 1 ml of sterile water containing 0.5 mg nalidixic acid, to eliminate the E. coli, and 1 mg apramycin, to select for the transformed actinomycete. 3 days after the addition of the antibiotic mix, 14 distinct sporulating colonies were observed on the transformation plate for Streptomyces sp. EN27. These colonies were picked off onto TSA supplemented with apramycin at 50 ug/ml. The plates were examined under blue light with an orange filter to detect fluorescence. Fluorescent colonies were a picked off and examined under a fluorescence microscope to confirm expression of egfp.

Visualisation of egfp-Expressing Streptomyces sp. EN27 using Laser Scanning Confocal Microscopy.

FIG. 5 shows the projection of an image stack of Streptomyces sp. EN27-egfp under the confocal microscope. 600× magnification was used with a 3× digital zoom to give an effective magnification of 1800×. A total of 51 optical slices were projected using Confocal Assistant version 4.0. The untransformed Streptomyces sp. EN27 exhibited no fluorescence in the green range of the spectrum. Neither the transformed nor the wild type Streptomyces sp. EN27 showed significant fluorescence in any other part of the spectrum that was tested.

Visualisation of Streptomyces sp. EN27-egfp.

Visualisation of the egfp tagged S. caviscabies/setonii in the seed used epifluorescence microscopy with blue light excitation from a mercury vapour lamp, and green light emission filters. After 24 hours the presence of the actinomycete was only detected in the embryo, and around the break in the seed husk where the embryo emerges from the seed. No fluorescence was observed on the outer seed husk, indicating that these cells were non-viable and no-longer expressing egfp, or more likely, these cells were washed away when the seeds were immersed in the freezing step during sectioning. FIG. 6 shows the actinomycete inhabiting the embryo tissue. The actinomycete was visualised using 465-495 nm wavelength excitation light with 515-555 nm emission filters, so only green light was visualised. Other filter sets including green excitation/red emission and UV excitation/blue emission were also tested to ensure the fluorescence was caused by EGFP. The plant tissue was visualised using UV excitation and blue emission as this produced the strongest autofluorescence in the plant cell walls. The image generated from the green light detection was digitally coloured green and the image generated from the blue light detection was digitally coloured red. The images were then overlaid using Confocal Assistant 4.0. It appeared that the actinomycete preferentially grows intracellularly in close proximity to the plant cell walls. It is also possible that this is intercellular growth and the microscope stage was slightly moved (down and to the right in the image) between the two image captures, as the actinomycete growth appears to mimic the shape of the plant cell wall in many places.

Visualisation of Streptomyces sp. EN27-egfp.

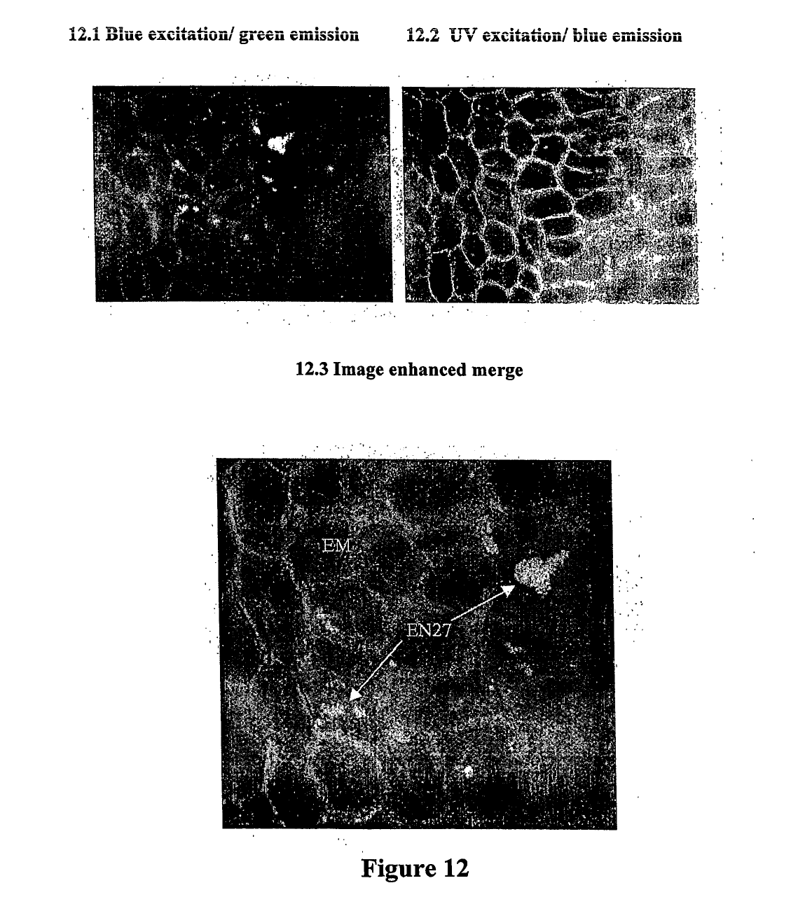

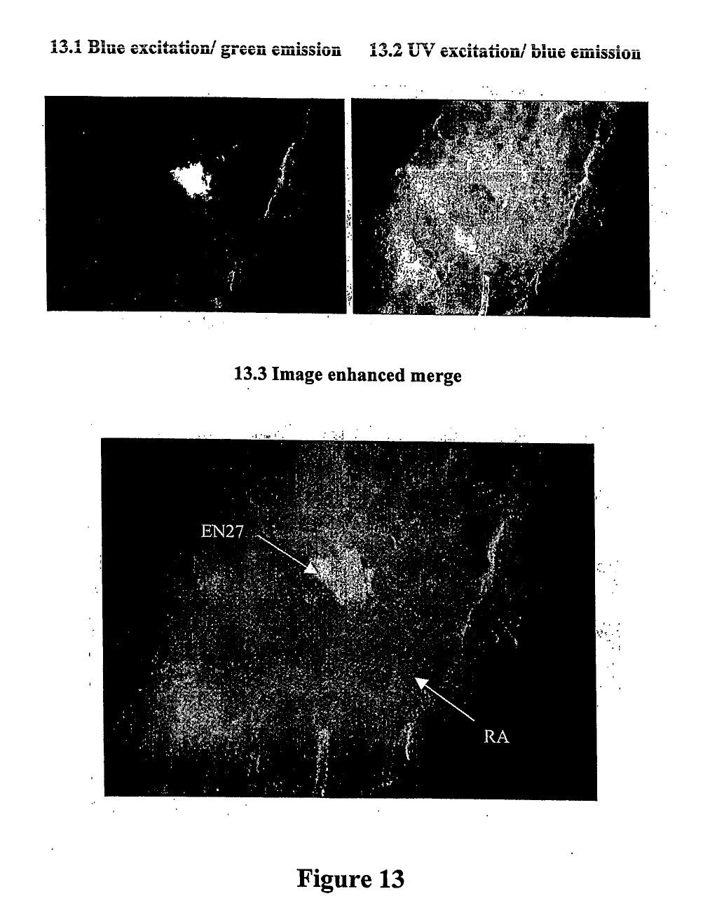

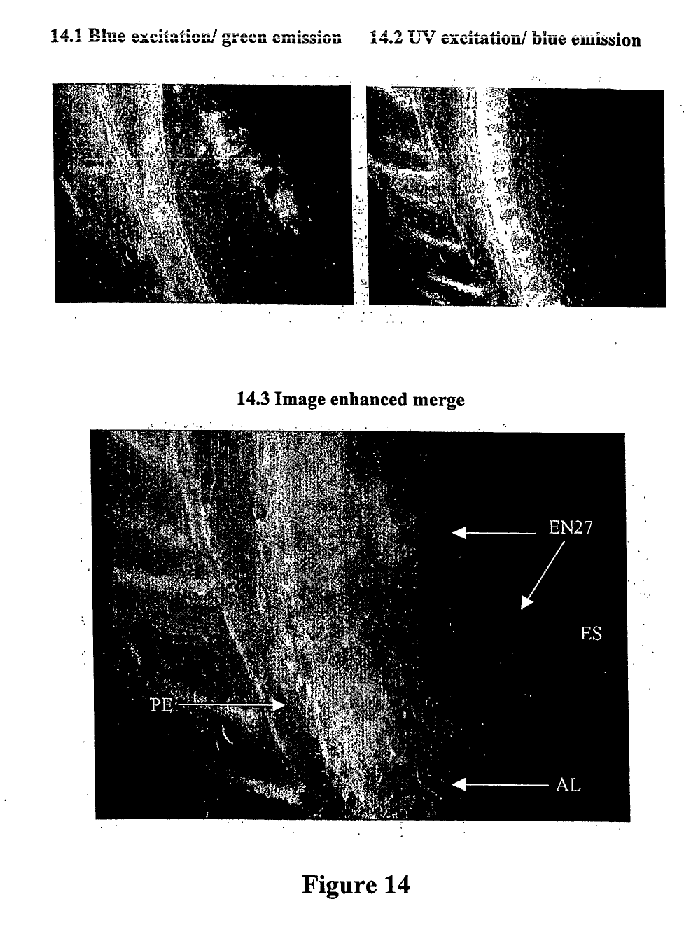

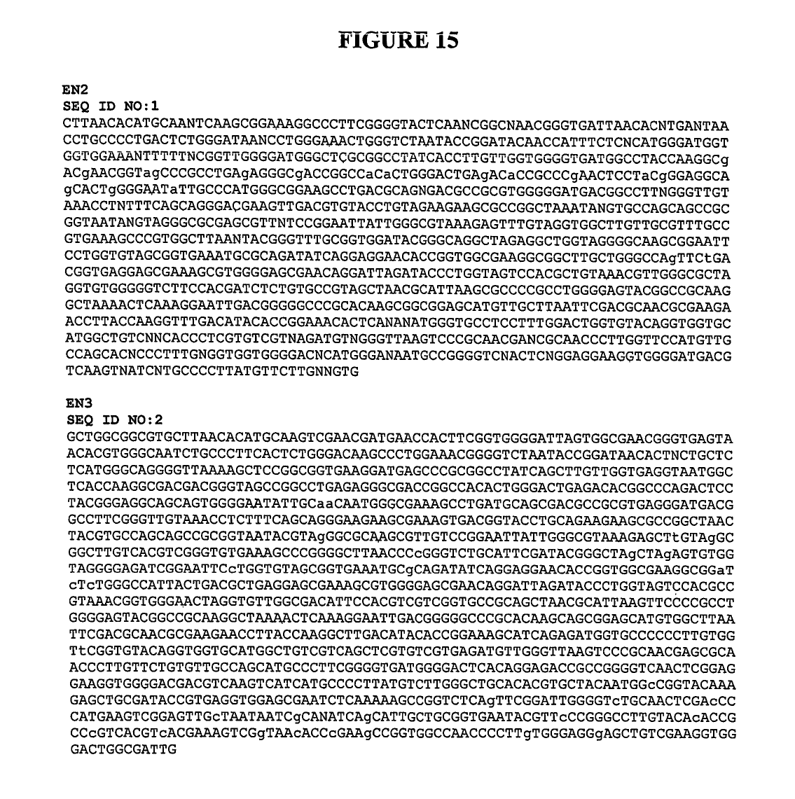

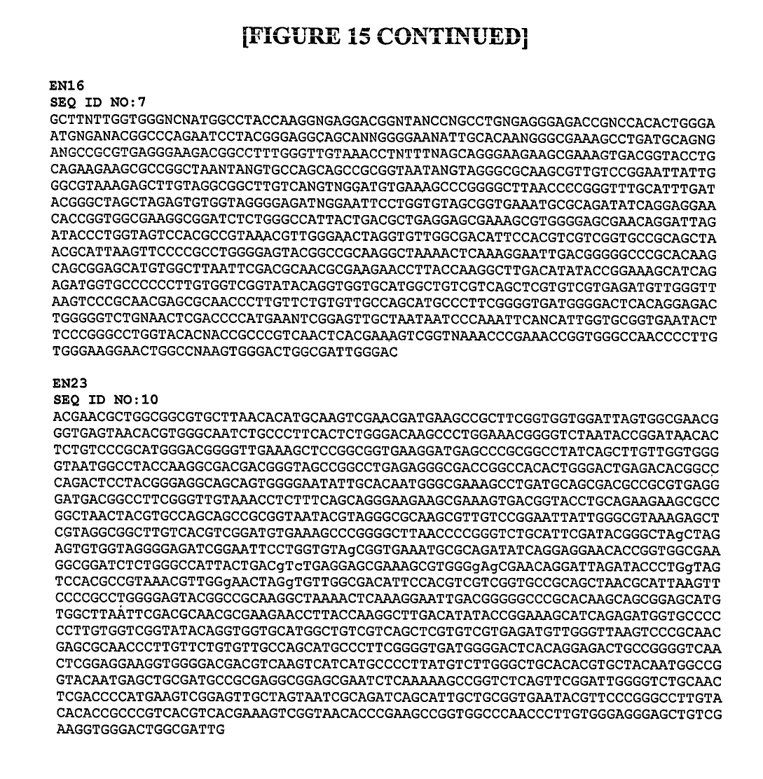

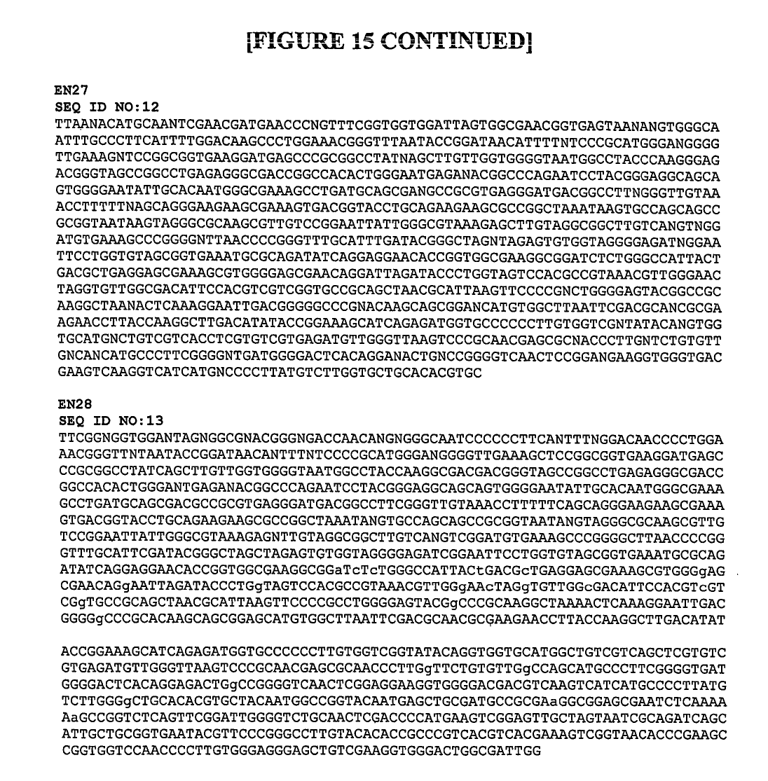

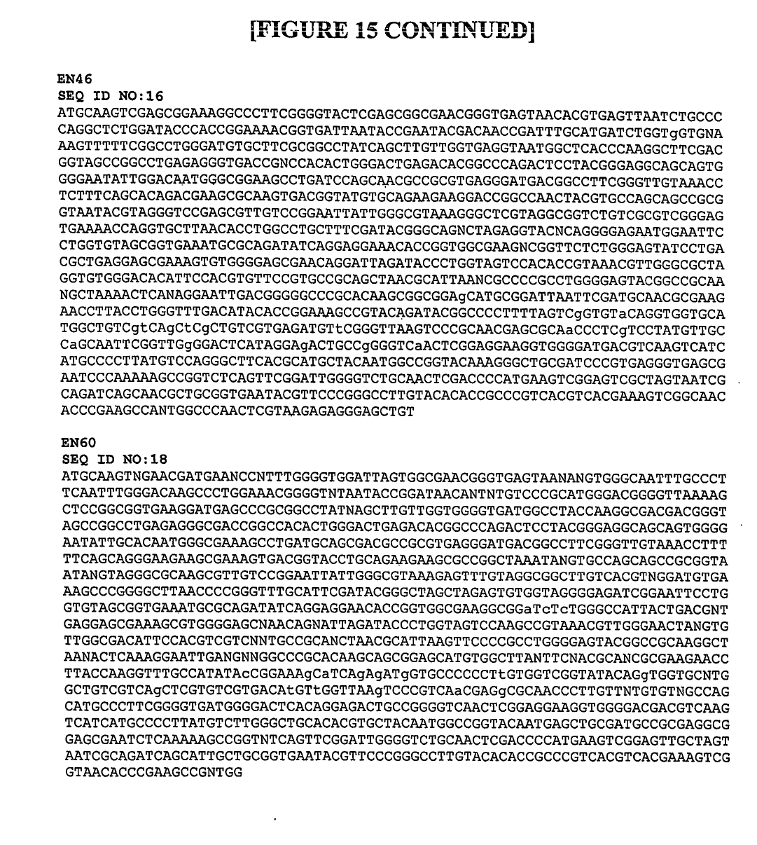

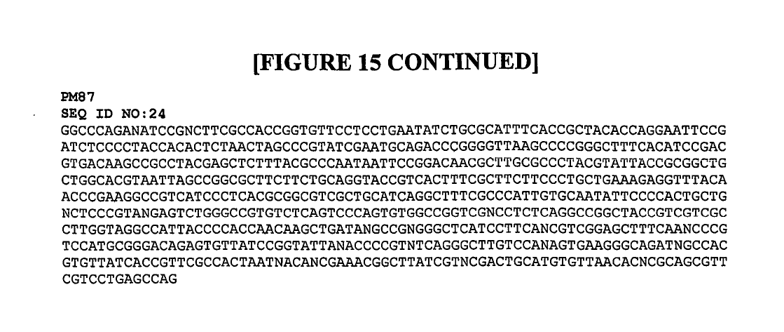

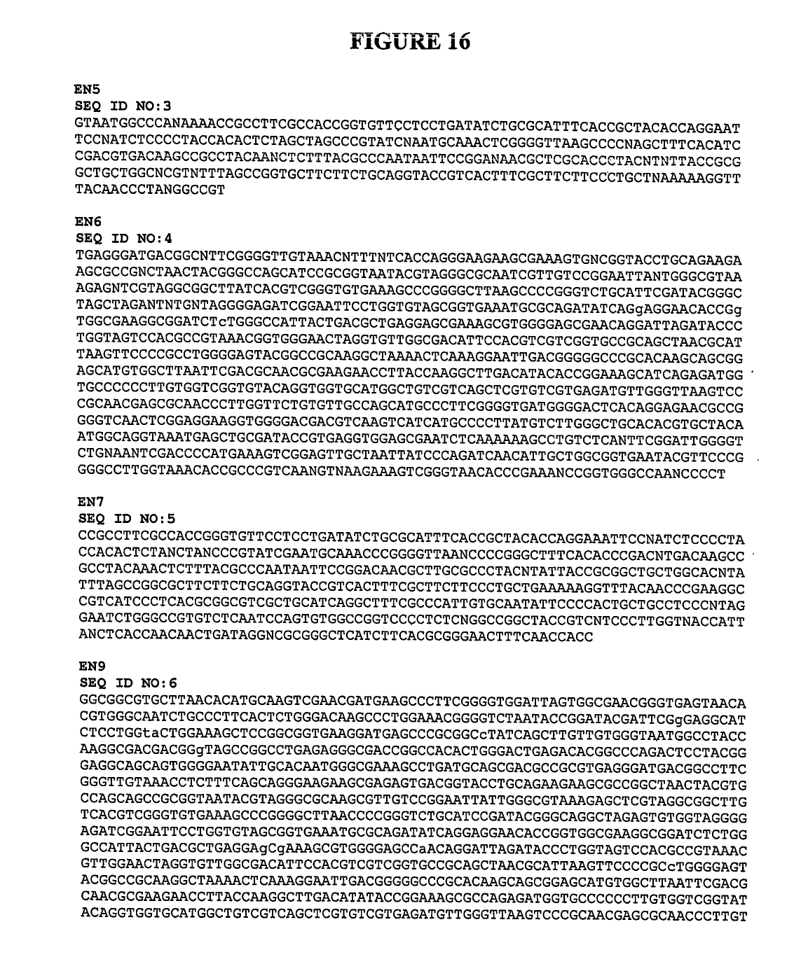

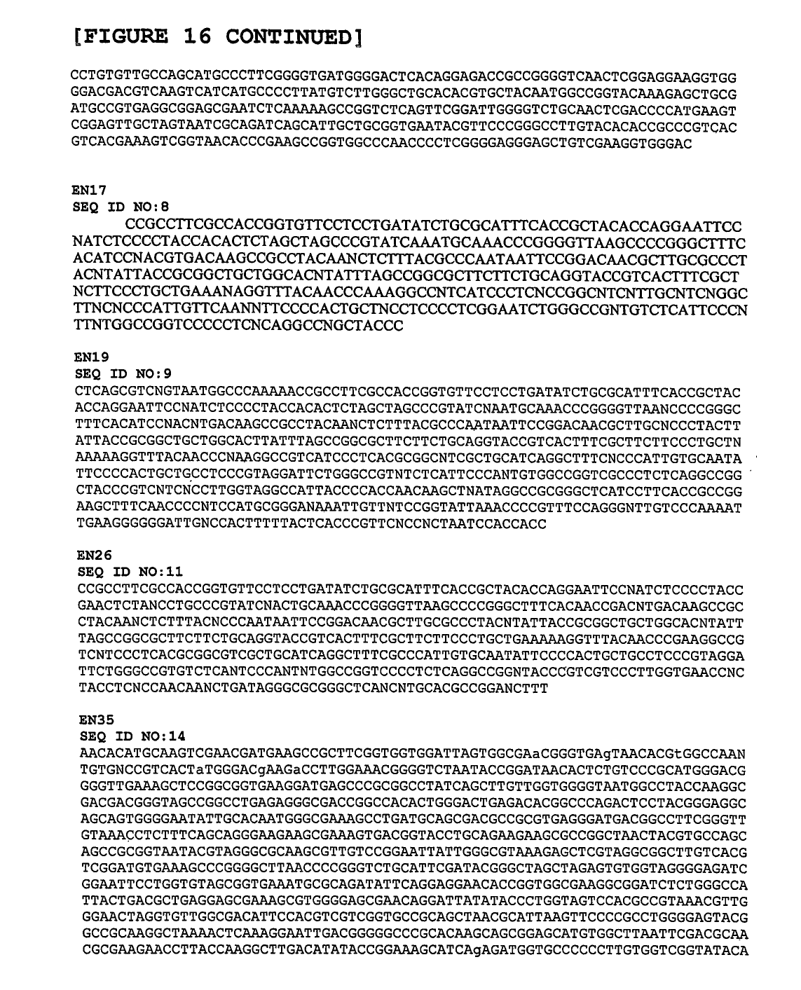

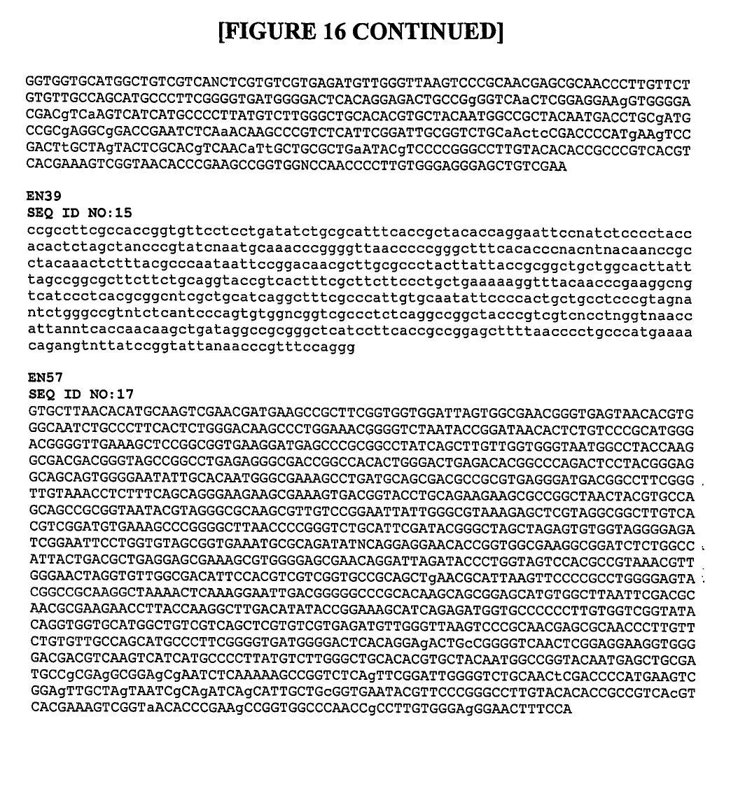

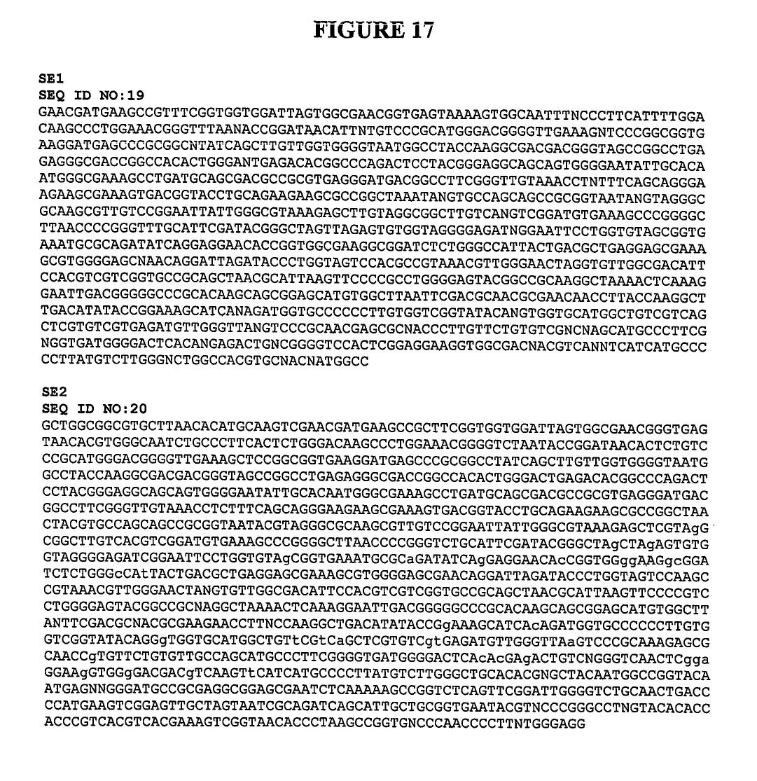

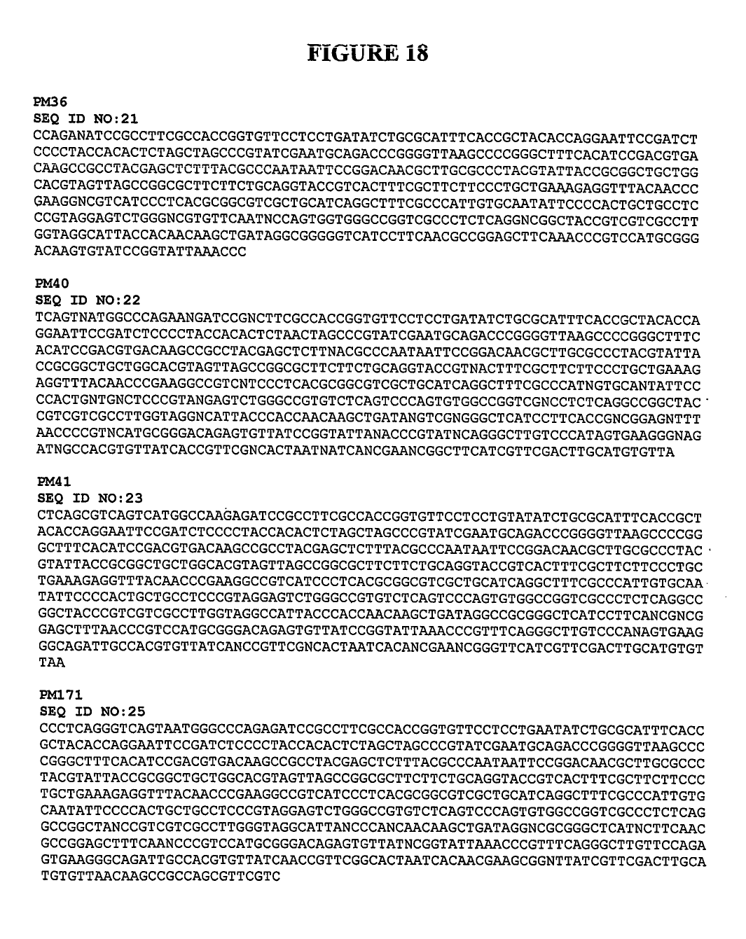

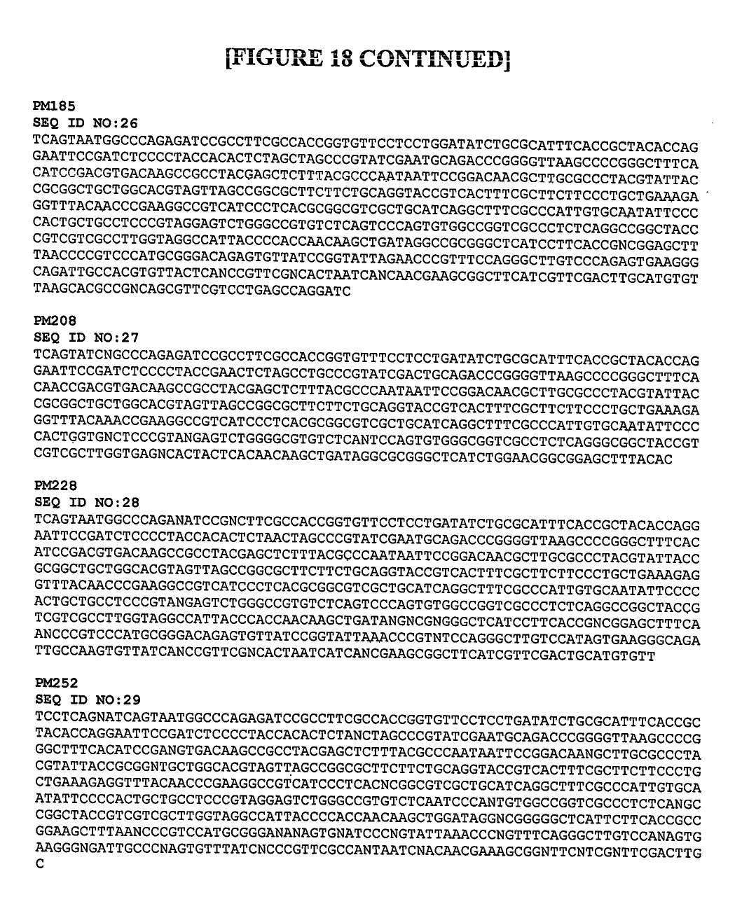

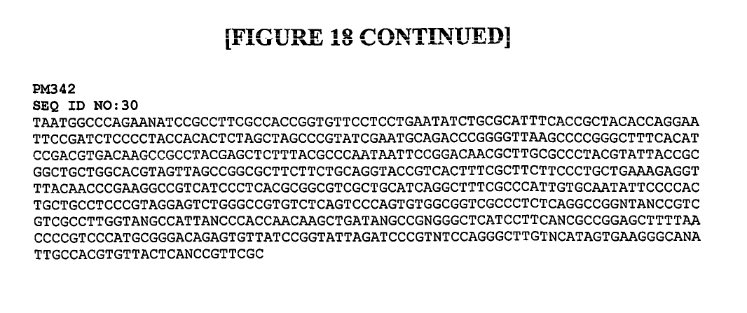

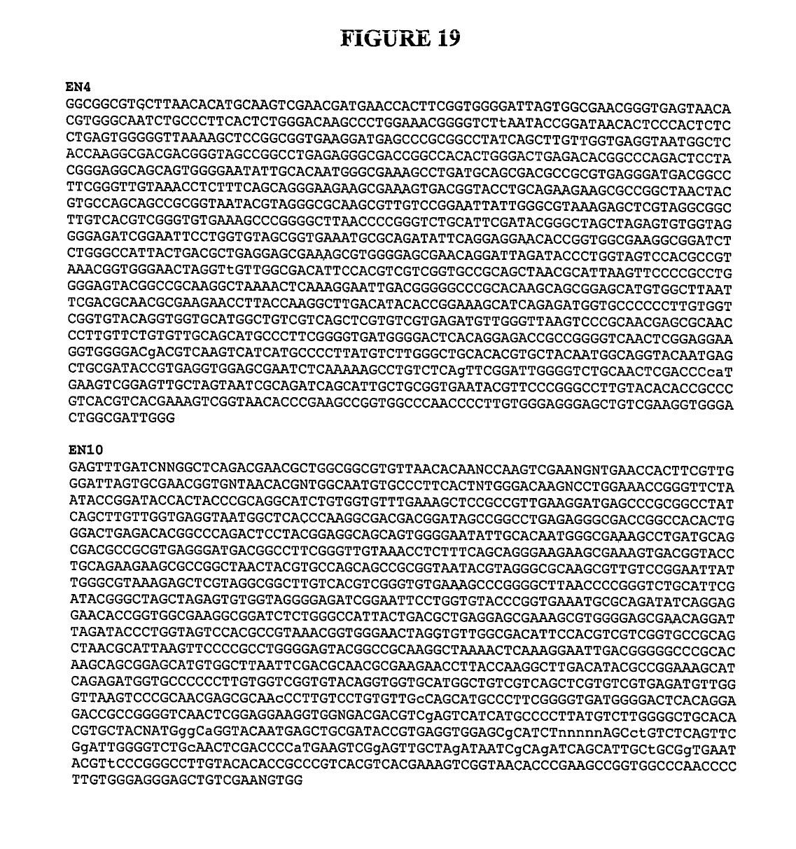

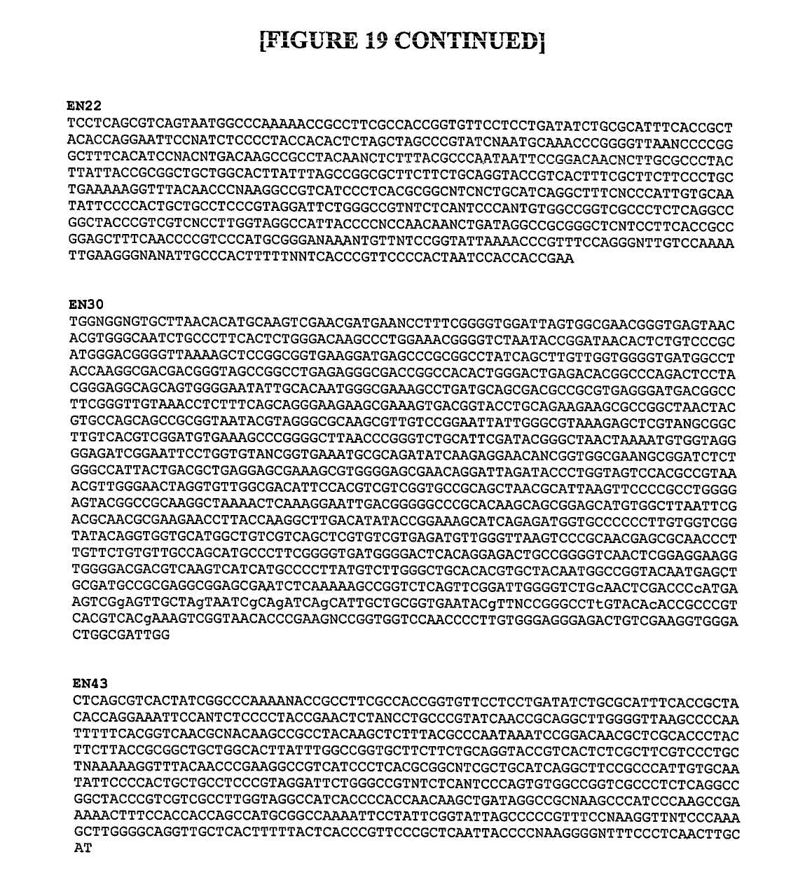

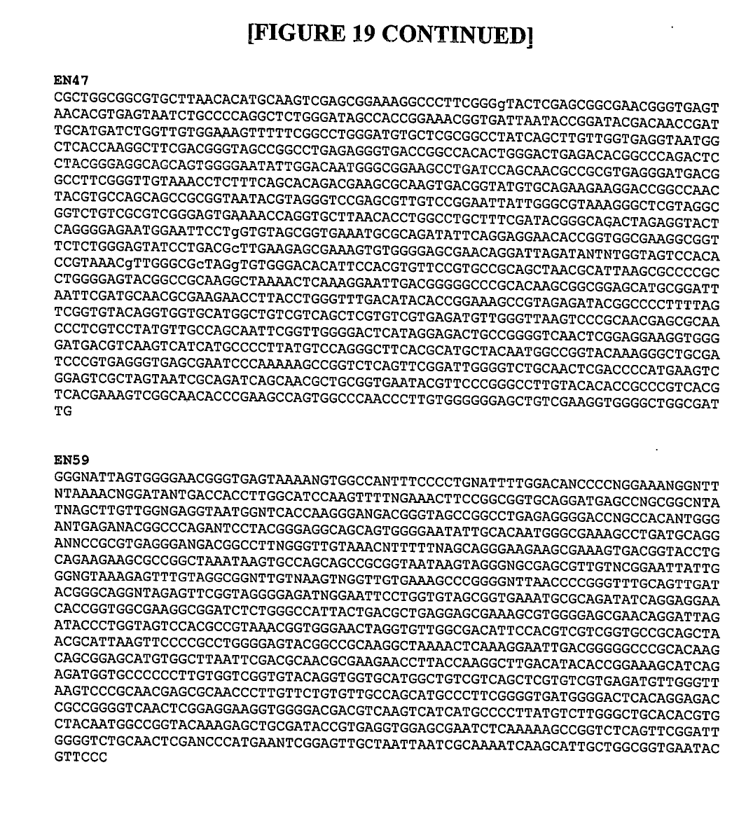

After 3 days egfp-expressing microcolonies of the actinomycete were seen more frequently in the embryo tissue of the seed than at 24 hours, indicating that the actinomycete was actively growing in the plant tissue. Examples of these microcolonies are shown in FIG. 12. Actinomycete microcolonies were also detected in the emerging radicle (young root) of the embryo, as seen in FIG. 13. After 3 days actinomycete growth was observed in the endosperm of the wheat seed, which was not observed at 24 hours (FIG. 14).

### EXAMPLE 15

### Production of the Auxin, Indole Acetic Acid, by Endophytic Actinomycete Strains

Assay Protocol

The ability of endophytic actinobacteria strains to produce indole-3-acetic acid (IAA) was assessed using a colorimetric assay using the protocol described by Glickmann and Dessaux (1995). Each actinomycete strain was grown in 10 ml Tryptone soya broth (TSB), supplemented with tryptophan at 200 mg.l⁻¹. At day 7 after inoculation, 750 ul samples of the culture broth were centrifuged at 12000 rpm to pellet the cells. 500 ul of this supernatant was then mixed with Salkowski reagent R1 (12 g.l⁻¹ FeCl₃ in 7.9M H₂SO₄) and allowed to stand for 20 min at room temperature. The optical density was measured at 530 nm for each sample using an Amersham Pharmacia Biotech Ultrospec 3100 pro spectrophotometer. The sample blank used to zero the instrument was the uninoculated culture medium mixed with Salkowski reagent R1, in the same manner as with the culture broths.

Results

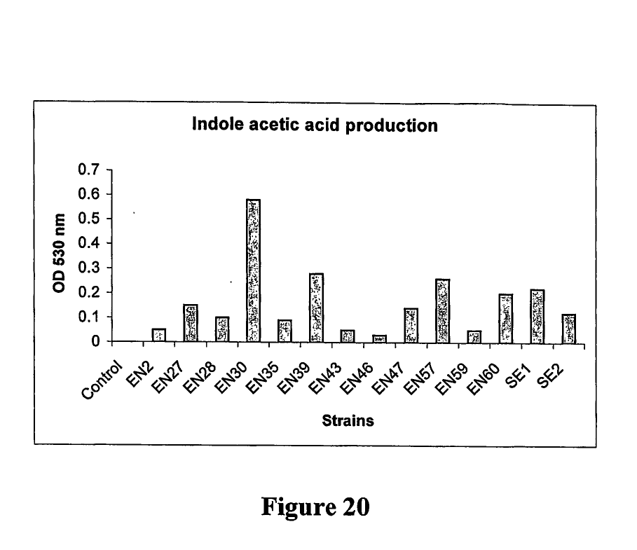

All the endophytic actinobacteria strains were able to produce detectable quantities of indole-3-acetic acid (IAA), as shown in the FIG. 20.

### EXAMPLE 16

### Wheat and Barley Seed Field Trails

Field trials were conducted to test the efficacy of a range of endophytic actinobacteria against Take-all and other cereal diseases.

Field trials were carried out using wheat and barley seed that were coated with the spores of a range of the actinobacterial endophytes. Trials were carried out in quadruplicate at each site.

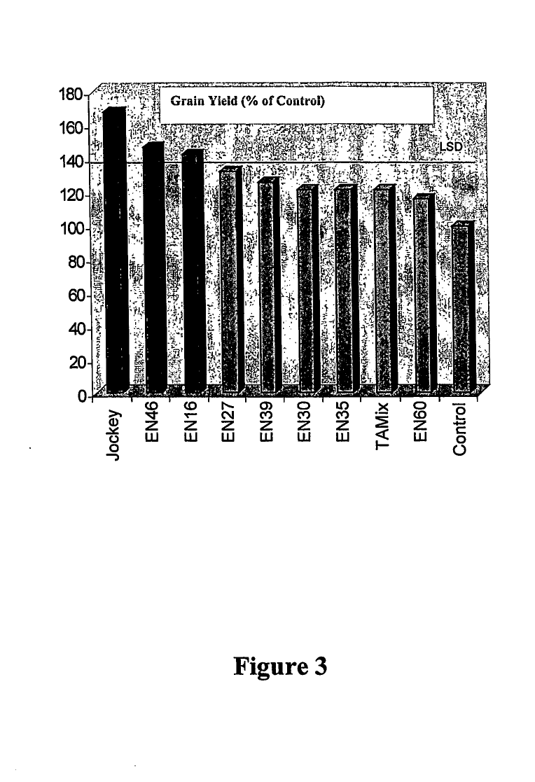

The field trials yielded a statistically significant result at the Alford site (Yorke Peninsula). Before the trial, soil DNA testing conducted at the South Australian Research and Development Institute (SARDI) showed high levels of Take-all present at this site. At this site substantial and significant yield increases occurred after seed treatment with a commercial control fungicide “Jockey”, and the actinobacterial endophytes Nocardioides sp. EN46 and Streptomyces triticum var. griseoviride EN16. Jockey is the currently the most effective chemical control agent so far developed for Take-all, and our endophyte treatments have resulted in statistically similar yields as was obtained with this treatment (FIG. 3).

In the three other sites over the state, with each treatment replicated 4 times, an important trend was observed. Overall, consistent yield increases were seen at nearly all sites treated with Streptomyces triticum var. griseoviride EN16, Streptomyces triticum EN27 and Nocardioides sp. EN46 (Table 8). Furthermore, at these sites, the commercial fungicide (Jockey) had no effect indicating the absence of disease. It is significant that in all these fields the endophyte treatments, particularly Streptomyces triticum var. griseoviride EN16, Streptomyces triticum EN27 and Nocardioides sp. EN46, substantially outperformed the commercial fungicide. The endophytes selected for these trials, however, produce the plant growth hormone indole-3-acetic acid (IAA), and are known plant growth promotion agents, based on our glasshouse trials.

Table 9 is a summary of field trials at sites with Rhizoctonia disease.

Table 10 is a summary of growth promotion trials at sites which had a low disease status.

### EXAMPLE 17

### Wheat and Barley Seed Field Trials—II

Field trials were also conducted in the 2003 growing season to further test the efficacy endophytic actinomycetes against a range of fungal pathogens including Gaeumannomyces graminis var. tritici (Take All), Fusarium spp. (Crown Rot), Rhizoctonia spp., Pythium spp. as well as assess the activity of the endophytic actinomycetes as growth promoters.

Seed treatment in each case was performed essentially as set out in Example 9. Specifically, for each actinomycete, a suspension of spores in sterile water was produced. For the “high” treatments, the spores were applied at about 10¹¹ spores per kilogram of seed. For the “low” treatments, spores were applied to the seed at about 10¹⁰ per kilogram of seed. The untreated seed or sham treated control was treated with sterile water. The treated and sham-treated (control) seed was air-dried under sterile conditions in a laminar flow hood prior to planting.

Trial sites were selected on the basis of disease history of the site and by evaluation using the soil DNA testing service (SARDI, Adelaide). For each inoculant treatment, 4 replicate fields plots of 1.5 μm width×20 m length were used in a randomised complete block design. Untreated seed was used in the control plots. For trials against Take-all disease, the commercial fungicide Jockey was used. Four replicates of the untreated control and the chemical fungicide were used at each trial site. The sowing rate was 85 kg per hectare. At harvest, grain yield was calculated as kg of seed per hectare and a comparison to the control was made.

The results of these field trials are presented in Tables 21 and 22.

### EXAMPLE 18

### Confirmation of Endophytic Root Colonization using T-RFLP

Seeds coated with EN2, EN27, EN46 and uncoated control seed were prepared as set out in Example 9.

The coated seeds were then planted and plants were harvested after 6 weeks of growth. Endophytic bacterial DNA was extracted from the roots of the putatively colonized wheat using the method described in Conn and Franco (Appl. Envir. Microbiol. 70: 1787-1794, 2004).

Partial 16S rRNA gene sequences were amplified from the endophytic DNA using the actinobacteria biased primers 243f (5′ GGA TGA GCC CGC CGC CTA 3′) and 5′TET (6-carboxy-2′,4,7,7′-tetrachlorofluorescein)-labelled 1492r (5′ TA CGG GTA CCT TGT TAC GAC TT 3′). Amplification was carried out according to Conn and Franco, supra. Single restriction digests of the 16S rRNA PCR products were performed using HinfI, HhaI and MboI (Promega) using 10 μl of the PCR reaction mixture for 16 to 18 hours to achieve complete digestion, and then stored at −20° C. The size of the terminal 16S rRNA gene fragments present in the restriction digestions were determined on an automated, Applied Biosystems 373 DNA sequencer, Stretch, using 1 μl of the restriction digest. Data was analysed using the GeneScan Analysis program V.3.1.2 (Applied Biosystems). From the GeneScan data the terminal restriction fragment (TRF) sizes present for each restriction enzyme was determined.

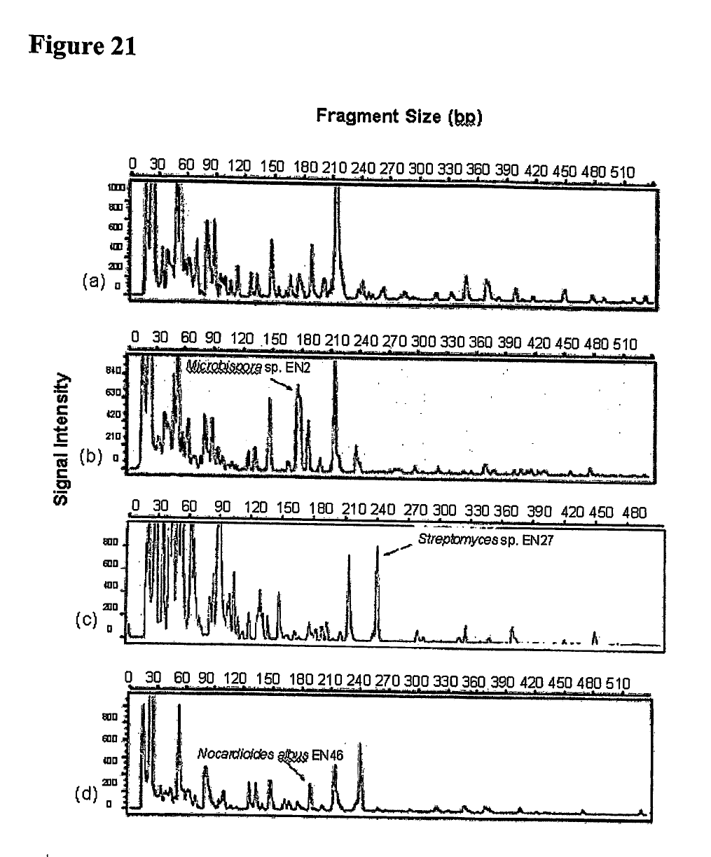

The T-RFLP profile obtained with HinfI for each of these plants is shown in FIG. 21; the annotated peaks indicate the fragment corresponding to the introduced actinobacterial endophyte. The calculated terminal restriction fragment of the EN27 16S product digested with HinfI is about 241 nucleotides in length, the EN46 product is about 179 bp in length and the EN2 the product is about 175 bp in length.

From the results it was observed that the HinfI fragment for Microbispora sp. EN2 increased by approximately two fold indicating that colonisation has occurred. The specific 241 bp HinfI fragment corresponding to Streptomyces sp. EN27 was not present in the uninoculated control which provided a good indication that colonisation has occurred.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 2

| | Strongest 16S rDNA sequence match (BLASTN) | | | | |
|---|---|---|---|---|---|
| | Accession | | | | |
| Isolate | No | Nearest match | Accession | Bits | % |
| EN2 | AY148073 | Streptosporangiacae | AF223347 | 1830 | 94 |
| | | str. PA147 | | | |
| | | Microbispora | U48988 | 1709 | 93 |
| | | amethystogenes | | | |
| EN3 | AY148077 | Streptomyces galilaeus | AB045878 | 2775 | 99 |
| EN4 | AY148080 | Streptomyces galilaeus | AB045878 | 2708 | 99 |
| EN6 | AY148085 | Streptomyces | AJ399481 | 1667 | 95 |
| | | pseudovenezuelae | | | |
| EN9 | AY148087 | Streptomyces bikiniensis | X79851 | 2573 | 98 |
| EN10 | AY148071 | Strptomyces fimbriatus | AB045868 | 2391 | 96 |
| | | Streptomyces sp.ASSF13 | AF012736 | 2002 | 95 |
| EN16 | AY148072 | Streptomyces caviscabies | AF112160 | 1994 | 95 |
| EN22 | AY291590 | Streptomyces peucetius | AB045887 | 2579 | 98 |
| EN23 | AY148074 | Streptomyces caviscabies | AF112160 | 2825 | 99 |
| EN27 | AY148075 | Streptomyces caviscabies | AF112160 | 1776 | 94 |
| EN28 | AY148076 | Streptomyces caviscabies | AF112160 | 2409 | 96 |
| EN30 | AY148078 | Streptomyces argenteolus | AB045872 | 2706 | 98 |
| EN35 | AY148079 | Streptomyces caviscabies | AF112160 | 2512 | 97 |
| EN43 | AY291589 | Micromonospora | X92626 | 2627 | 98 |
| | | yulongensis | | | |
| EN46 | AY148081 | Nocardioides albus | X53211 | 2516 | 98 |
| EN47 | AY148082 | Nocardioides albus | X53211 | 2769 | 99 |
| EN57 | AY148083 | Streptomyces caviscabies | AF112160 | 2684 | 99 |
| EN59 | AY148084 | Streptomyces galilaeus | AB045878 | 1879 | 95 |
| EN60 | AY148086 | Streptomyces argenteolus | AB045872 | 2375 | 96 |
| SE1 | AY148088 | Streptomyces caviscabies | AF112160 | 1879 | 95 |
| SE2 | AY148089 | Streptomyces caviscabies | AF112160 | 2528 | 97 |

TABLE 3

| | Preliminary Identification based on 16S rDNA sequencing |
|---|---|
| Isolate | (showing % similarity to nearest matching sequence) |
| EN5 | Streptomyces caviscabies/Str. setonii (92%) |
| EN7 | Streptomyces lincolnesis (93%) |
| EN17 | Streptomyces caviscabies (90%) |
| EN19 | Streptomyces caviscabies/Str. setonii (92%) |
| EN26 | Streptomyces peruviensis (94%) |
| EN39 | Streptomyces galilaeus (93%) |
| EN41 | Micromonospora yulongensis (92%) |
| EN42 | Micromonospora peucetica (91%) |
| PM 20 | Streptomyces caviscabies/Str. setonii (93%) |
| PM 22 | Streptomyces caviscabies sp. (96%) |
| PM 23 | Streptomyces caviscabies (93%) |
| PM 35 | Tsukamurella tyrosinovorans D-1498 (96%) |
| PM 36 | Streptomyces caviscabies/Str. setonii (95%) |
| PM 40 | Streptomyces caviscabies/Str. setonii (93%) |
| PM 41 | Streptomyces caviscabies/Str. setonii (96%) |
| PM 87 | Streptomyces caviscabies/Str. setonii (94%) |
| PM 89 | Streptomyces lincolnensis (97%) |
| PM 124 | Tsukamurella sp. IM-7430 (97%) |
| PM 144 | Streptomyces caviscabies/Str. setonii (97%) |
| PM 171 | Streptomyces caviscabies/Str. setonii (93%) |
| PM 185 | Streptomyces caviscabies/Str. setonii (98%) |
| PM 208 | Streptomyces caviscabies/Str. setonii (95%) |
| PM 228 | Streptomyces caviscabies/Str. setonii (96%) |
| PM 239 | Tsukamurella tyrosinovorans D-1498 (96%) |
| PM 247 | S. caviscabies (95%) |
| PM 252 | Streptomyces caviscabies/Str. setonii (92%) |
| PM 301 | Streptomyces caviscabies (93%) |
| PM 342 | Streptomyces caviscabies/Str. setonii (96%) |
| SC 19 | Micromonospora fulvoviolaceus (97%) |

TABLE 4

| | | | | | | | Carbohydrate | | |
|---|---|---|---|---|---|---|---|---|---|
| Isolate | Spore coloration | | | | | | utilization | | |
| number | ISP2 | ISP3 | ISP4 | ISP5 | ISP7 | NUTRIENT AGAR | gluc | fruc | suc |
| EN 19 | cream | green | white | white | black | white | + | + | + |
| EN 27 | cream | white | white | brown | brown | white | + | + | + |
| EN 35 | cream | white | white | brown | brown | white | + | + | − |
| EN 57 | cream | white | white | brown | brown | white | + | + | + |
| EN 28 | cream | cream | white | black | brown | white | + | + | + |
| SE 1 | white | white | white | light brown | dark brown | white | + | + | + |
| SE 2 | white | white | white | light brown | dark brown | white | + | + | + |
| PM 40 | white | white | white | light brown | dark brown | white | + | + | + |
| PM 41 | cream | white | white | brown | dark brown | white | + | + | + |
| PM 228 | cream | white | white | brown | dark brown | white | + | − | − |
| PM 36 | cream | white | white | dark brown | white | white | + | + | + |
| PM 87 | cream | white | white | dark brown | white | white | + | + | + |
| PM 252 | cream | white | white | dark brown | white | white | + | + | − |
| PM 171 | cream | white | white | dark brown | pale green | white | + | + | − |

| Isolate | Carbohydrate utilization | | | | | | | | | | | |
|---|---|---|---|---|---|---|---|---|---|---|---|---|
| number | mantol | mal | inos | gal | man | lac | cell | dul | xyl | rham | raff | arab |
| EN 19 | + | + | − | + | + | − | − | − | + | − | − | − |
| EN 27 | + | + | − | + | + | + | − | − | + | − | − | − |
| EN 35 | − | + | − | − | + | − | − | − | + | − | − | − |
| EN 57 | + | + | − | + | − | + | − | − | + | − | − | − |
| EN 28 | + | + | − | + | + | + | − | − | − | − | + | − |
| SE 1 | + | + | − | − | + | − | − | − | − | − | − | − |
| SE 2 | + | + | − | + | + | − | − | − | − | − | − | − |
| PM 40 | + | + | − | − | + | + | − | − | + | − | − | − |
| PM 41 | − | − | − | + | + | + | − | − | − | − | − | − |
| PM 228 | − | + | − | + | − | + | − | − | + | − | − | − |
| PM 36 | + | + | − | + | + | − | − | − | − | − | − | − |
| PM 87 | + | − | − | − | + | − | − | − | − | − | − | − |
| PM 252 | − | − | − | + | − | − | − | − | + | − | − | − |
| PM 171 | + | + | + | + | + | − | − | − | − | − | − | − |

KEY:
gluc—glucose;
fruc—fructose;
suc—sucrose;
mantol—mannitol;
mal—maltose;
inos; inositol;
gal—galactose;
man—mannose;
lac—lactose;
cell—cellulose;
dul—dulcitol;
xyl—xylose;
rham—rhamnose;
raff—raffinose and
arab—arabinose.

TABLE 5

| | | | | | | | Biochemical analysis | |
|---|---|---|---|---|---|---|---|---|
| Isolate | Soluble pigmentation | | | | | | Starch | Gelatin |
| Number | ISP2 | ISP3 | ISP4 | ISP5 | ISP7 | NUTRIENT AGAR | Hydrolysis | Digestion |
| EN 19 | dark brown | — | — | black | black | — | +++ | + |
| EN 27 | brown | — | — | black | black | — | +++ | + |
| EN 35 | brown | — | — | black | black | — | ++ | + |
| EN 57 | brown | — | — | black | black | — | +++ | + |
| EN 28 | brown | dark | — | black | black | — | ++ | + |
| | | | cream | | | | | |
| SE 1 | dark brown | brown | — | light | dark brown | — | ++ | + |
| | | | | brown | | | | |
| SE 2 | dark brown | brown | — | light | dark brown | — | ++ | + |
| | | | | brown | | | | |
| PM 40 | dark brown | brown | — | Light | dark brown | — | ++ | + |
| | | | | brown | | | | |
| PM 41 | dark brown | brown | Light | black | black | — | ++ | + |
| | | | brown | | | | | |
| PM 228 | dark brown | brown | light | black | black | — | + | + |
| | | | brown | | | | | |
| PM 36 | light brown | — | — | black | brown | — | +++ | + |
| PM 87 | light brown | — | — | black | brown | — | + | + |
| PM 252 | light brown | — | — | black | brown | — | ++ | + |
| PM 171 | brown | — | — | brown | black | — | + | + |

| | Biochemical analysis | | | | | | | |
|---|---|---|---|---|---|---|---|---|
| Isolate | Nitrate | Urea | H2S | Max naCl | | Streptomycin | Litmus Milk Test | |
| Number | Reduction | Utilization | Production | Tolerance | Min pH* | Inhibition** | Pepton^{N} | Coagul^{N} |
| EN 19 | − | + | − | 7% | pH 5 | 1.5 μg/ml | − | + |
| EN 27 | + | + | − | 7% | pH 4 | 1 μg/ml | − | − |
| EN 35 | − | + | − | 7% | pH 4 | 1.5 μg/ml | + | − |
| EN 57 | − | + | − | 6% | pH 4 | 1.5 μg/ml | + | − |
| EN 28 | − | + | + | 7% | pH 4 | 1 μg/ml | + | − |
| SE 1 | − | + | − | 7% | pH 3 | 1.5 μg/ml | − | + |
| SE 2 | − | + | − | 7% | pH 3 | 1 μg/ml | − | − |
| PM 40 | + | + | − | 8% | pH 3 | 1 μg/ml | − | + |
| PM 41 | + | + | + | 8% | pH 4 | 1.5 μg/ml | − | − |
| PM 228 | − | + | − | 7% | pH 4 | 1 μg/ml | − | − |
| PM 36 | + | + | − | 8% | pH 4 | 1 μg/ml | + | − |
| PM 87 | + | − | + | 7% | pH 3 | 1 μg/ml | + | − |
| PM 252 | − | + | + | 7% | pH 4 | 2 μg/ml | − | − |
| PM 171 | − | + | + | 6% | pH 4 | 1 μg/ml | − | + |

Key:
In Starch Hydrolysis
+ minimum hydrolysis;
++ intermediate hydrolysis
+++ complete hydrolysis

TABLE 6

| | Spore colouration | | | | | | | | | |
|---|---|---|---|---|---|---|---|---|---|---|
| | | | | | | NUTRIENT | Carbohydrate utilization | | | |
| isolates | ISP2 | ISP3 | ISP4 | ISP5 | ISP7 | AGAR | gluc | fruc | suc | mantol |
| EN 16 | green | green | grey | white | grey | white | + | − | + | − |
| EN 17 | grey | white | grey | white | white | white | + | + | + | + |
| PM 144 | green | white | white | white | green | white | + | + | + | + |
| PM 185 | grey | white | white | white | white | white | + | + | + | − |
| PM 208 | grey | white | white | white | white | white | + | + | − | + |
| PM 342 | white | white | white | white | grey | white | + | + | − | − |
| S. caviscabies | cream | white | white | white | white | white | + | + | + | + |
| (ATCC 51928) | | | | | | | | | | |
| S. setonil | cream | white | white | white | white | white | + | + | + | + |
| (ATCC 25497) | | | | | | | | | | |

| | Carbohydrate utilization | | | | | | | | | | |
|---|---|---|---|---|---|---|---|---|---|---|---|
| isolates | mal | inos | gal | man | lac | cell | dul | xyl | rham | raff | arab |
| EN 16 | + | − | + | + | − | − | − | − | − | − | − |
| EN 17 | + | − | + | + | + | − | − | + | − | − | − |
| PM 144 | + | + | + | + | + | − | − | − | − | + | − |
| PM 185 | − | − | + | + | − | − | − | − | − | − | − |
| PM 208 | − | − | − | + | + | − | − | − | − | − | − |
| PM 342 | + | − | + | + | − | − | − | − | − | − | − |
| S. caviscabies | + | + | + | + | + | − | − | + | − | − | − |
| (ATCC 51928) | | | | | | | | | | | |
| S. setonil | + | + | + | + | + | − | − | + | − | − | − |
| (ATCC 25497) | | | | | | | | | | | |

KEY:
gluc—glucose;
fruc—fructose;
suc—sucrose;
mantol—mannitol;
mal—maltose;
inos; inositol;
gal—galactose;
man—mannose;
lac—lactose;
cell—cellutose;
dul—dulcitol;
xyl—xylose;
rham—rhamnose;
raff—raffinose and
arab—arbinose.

TABLE 7

| | soluble pigmentation | | | | | | Biochemical analysis | | |
|---|---|---|---|---|---|---|---|---|---|
| Isolate | | | | | | NUTRIENT | Starch | Gelatin | Nitrate |
| Number | ISP2 | ISP3 | ISP4 | ISP5 | ISP7 | AGAR | Hydrolysis | Digestion | Reduction |
| EN 16 | brown | — | — | — | grey | — | + | + | + |
| EN 17 | light brown | — | brown | light brown | light brown | — | + | + | + |
| PM 144 | brown | — | green | brown | — | — | +++ | + | + |
| PM 185 | light brown | — | — | — | light brown | — | ++ | + | + |
| PM 208 | brown | — | — | — | — | — | ++ | + | − |
| PM 342 | — | — | — | — | — | — | +++ | − | + |
| S. caviscabies | — | light orange | — | — | — | — | +++ | + | + |
| (ATCC 51928) | | | | | | | | | |
| S.setonil | — | — | — | — | — | — | +++ | − | − |
| (ATCC 25497) | | | | | | | | | |

| | Biochemical analysis | | | | | | |
|---|---|---|---|---|---|---|---|
| Isolate | Urea | H2S | Max NaCl | | Streptomycin | Litmus Milk Test | |
| Number | Utilization | Production | Tolerance | Min pH | Inhibition** | Peptonisation | Coagulation |
| EN 16 | + | − | 7% | pH 5 | 1 μgm/l | − | + |
| EN 17 | + | − | 6% | pH 5 | 1 μg/ml | − | − |
| PM 144 | − | + | 6% | pH 5 | 1 μg/ml | − | − |
| PM 185 | + | − | 9% | pH 5 | 1 μg/ml | − | − |
| PM 208 | − | − | 9% | PH 5 | 1 μg/ml | − | + |
| PM 342 | + | − | 6% | pH 5 | 1 μg/ml | − | − |
| S. caviscabies | + | − | 7% | pH 5 | 1 μg/ml | − | + |
| (ATCC 51928) | | | | | | | |
| S.setonil | + | + | 7% | pH 5 | 1 μg/ml | + | − |
| (ATCC 25497) | | | | | | | |

Key:
In Starch Hydrolysis
+ minimum hydrolysis;
++ intermediate hydrolysis
+++ complete hydrolysis.

TABLE 8

| | Sandilands | Freeling | Mallala | Mean |
|---|---|---|---|---|
| EN16 | 111 | 103 | 106 | 6.67 |
| EN46 | 114 | 100 | 104 | 6.00 |
| EN27 | 103 | 103 | 104 | 3.33 |
| EN60 | 99 | 105 | 100 | 1.33 |
| EN39 | 103 | 103 | 98 | 1.33 |
| EN35 | 99 | 102 | 102 | 1.00 |
| TAMix | 92 | 102 | 106 | 0.00 |
| Control | 100 | 100 | 100 | 0.00 |
| Jockey | 98 | 102 | 99 | −0.33 |
| EN30 | 101 | 98 | 99 | −0.67 |

TABLE 9

| Treatment | *Alford | #Sandilands | *Haslam | #Wudinna | #Waddikie | Ave |
|---|---|---|---|---|---|---|
| EN9 | 109 | 103 | 100 | 102 | 109 | 105 |
| EN23 | 100 | 118 | 101 | 97 | 109 | 105 |
| EN27 | 91 | 113 | 102 | 103 | 110 | 104 |
| EN28 | 96 | 109 | 100 | 102 | 105 | 102 |
| EN60 | 91 | 97 | 100 | 101 | 112 | 100 |
| Rmix1 EN2_9_23 | 117 | 99 | 100 | 100 | 103 | |
| Rmix2 EN9_27_28 | 109 | 103 | 96 | 103 | 111 | 104 |
| Rmix3 EN39_46 | nd | nd | 99 | 104 | 113 | 105 |
| MR1 | 100 | 105 | nd | nd | nd | 103 |
| Jockey | 109 | 103 | 104 | 97 | 110 | 104 |
| Untreated | 100 | 100 | 100 | 100 | 100 | 100 |

nd = not done;
*barley #wheat

TABLE 10
Locations: Frances and Mundulla, SE of SA

| | |
|---|---|
| Frances | Mundulla |

| Treatment | kg/ha | % Control | kg/ha | % Control |
|---|---|---|---|---|
| EN3 | 2219 | 94 | 2275 | 107 |
| EN27 | 2235 | 95 | 2222 | 104 |
| PM87 | 2291 | 97 | 2313 | 108 |
| PM330 | 2281 | 97 | 2176 | 102 |
| GPMix | 2192 | 93 | 2280 | 107 |
| Untreated | 2356 | 100 | 2134 | 100 |

GPMix = EN6, EN27, PM87, PM330

TABLE 11

| | Ggt-B100 | | Ggt-C3201 | | Ggt-17916 | |
|---|---|---|---|---|---|---|
| EN | 4 days | 7 days | 4 days | 7 days | 4 days | 7 days |
| 1 | − | + | − | +/− | − | +/− |
| 2 | − | ++ | − | − | − | − |
| 3 | − | + | − | +/− | +/− | +/− |
| 4 | − | + | − | + | − | +/− |
| 5 | − | − | − | − | − | +/− |
| 6 | + | +/− | − | − | − | − |
| 7 | − | − | − | + | − | +/− |
| 8 | +++ | +++ | +++ | +++ | +++ | +++ |
| 9 | − | − | − | − | − | − |
| 10 | − | − | − | + | − | + |
| 11 | + | + | − | − | − | − |
| 12 | ND | ND | ND | ND | ND | ND |
| 13 | +++ | +++ | +++ | +++ | +++ | +++ |
| 14 | ND | ND | ND | ND | ND | ND |
| 15 | − | − | − | + | − | − |
| 16 | + | ++ | + | ++ | + | ++ |
| 17 | +/− | ++ | ++ | +++ | ++ | +++ |
| 18 | − | − | − | +/− | − | + |
| 19 | +++ | +++ | +++ | +++ | +++ | +++ |
| 20 | − | − | − | − | − | − |
| 21 | ND | ND | ND | ND | ND | ND |
| 22 | − | +/− | − | +/− | − | − |
| 23 | − | + | − | + | − | + |
| 24 | ND | ND | ND | ND | ND | ND |
| 25 | − | − | +/− | ++ | − | + |
| 26 | ++ | +++ | +++ | +++ | +++ | +++ |
| 27 | +++ | +++ | − | + | − | + |
| 28 | − | + | + | +++ | +++ | +++ |
| 29 | − | − | − | +/− | − | − |
| 30 | − | + | − | + | +/− | +/− |
| 31 | − | − | − | +/− | − | − |
| 32 | − | − | − | − | − | − |
| 33 | − | ++ | +++ | ++ | +++ | +++ |
| 34 | − | − | − | − | − | +/− |
| 35 | − | − | + | ++ | − | − |
| 36 | − | − | − | + | − | +/− |
| 37 | − | − | − | + | − | − |
| 38 | − | − | − | + | − | +/− |
| 39 | − | + | − | +/− | − | − |
| 40 | − | − | − | + | − | − |
| 41 | − | − | − | − | − | − |
| 42 | − | − | − | − | − | − |
| 43 | − | − | − | − | − | − |
| 44 | − | − | − | − | − | − |
| 45 | − | + | +/− | + | +/− | +/− |
| 46 | − | + | − | +/− | − | − |
| 47 | − | − | − | − | − | − |
| 48 | − | − | + | ++ | +/− | +/− |
| 49 | − | +/− | +/− | +/− | +/− | +/− |
| 50 | − | + | + | + | +/− | + |
| 51 | +++ | +++ | +++ | +++ | +++ | +++ |
| 52 | − | + | − | +/− | − | − |
| 53 | +++ | ++ | − | + | − | +/− |
| 54 | + | + | − | +/− | − | + |
| 55 | + | + | + | + | +/− | + |
| 56 | +++ | +++ | +++ | +++ | ++ | +++ |
| 57 | ++ | ++ | +++ | +++ | +++ | +++ |
| 58 | +++ | +++ | +++ | +++ | +++ | +++ |
| 59 | − | + | + | + | − | +/− |
| 60 | − | − | − | − | − | +/− |
| 61 | ++ | +++ | +++ | +++ | +++ | +++ |

Strength of antagonism of each actinomycete isolate against fungal wheat pathogens, measured after 4 and 7 days of incubation.
Very strong antagonism (30 ++),
Strong antagonism (++),
Moderate antagonism (+),
Weak antagonism (+/−),
No antagonism (−),
Not done (ND).

TABLE 12

R. solani

| EN | 4 days | 7 days |
|---|---|---|
| 2 | +/− | − |
| 3 | +/− | ++ |
| 4 | + | + |
| 5 | ++ | +++ |
| 6 | − | + |
| 7 | +/− | +/− |
| 9 | + | + |
| 6 | + | ++ |
| 17 | +++ | +++ |
| 19 | +++ | +++ |
| 23 | − | + |
| 26 | +++ | +++ |
| 27 | + | ++ |
| 28 | ++ | +++ |
| 30 | +/− | +/− |
| 35 | + | +/− |
| 39 | + | +/− |
| 43 | − | +/− |
| 46 | ++ | + |
| 47 | +/− | + |
| 57 | +++ | +++ |
| 59 | ++ | ++ |
| 60 | ++ | +++ |

Strength of antagonism of each actinomycete isolate against fungal wheat pathogens, measured after 4 and 7 days of incubation.
Very strong antagonism (+++),
Strong antagonism (++),
Moderate antagonism (+),
Weak antagonism (+/−),
No antagonism (−),
Not done (ND).

TABLE 13

| | |
|---|---|
| Pythium sp. | Pythium sp. |
| KAP3 | BH40 |

| EN | 4 days | 7 days | 4 days | 7 days |
|---|---|---|---|---|
| 2 | − | − | − | − |
| 3 | + | + | + | +/− |
| 4 | − | − | − | − |
| 5 | ++ | ++ | +/− | +/− |
| 6 | +/− | − | +/− | − |
| 7 | + | +/− | +/− | +/− |
| 9 | + | + | +/− | +/− |
| 16 | + | + | + | +/− |
| 17 | +++ | +++ | ++ | ++ |
| 19 | +++ | +++ | +++ | +++ |
| 22 | − | − | +/− | − |
| 23 | + | + | + | + |
| 26 | ++ | ++ | +++ | +++ |
| 27 | + | + | + | + |
| 28 | +++ | +++ | ++ | + |
| 35 | +/− | − | − | − |
| 39 | +/− | +/− | − | − |
| 44 | − | − | − | − |
| 46 | − | +/− | + | +/− |
| 47 | − | − | − | +/− |
| 57 | +/− | +/− | + | + |
| 59 | + | +/− | + | + |
| 60 | + | + | +/− | ++ |

Strength of antagonism of each actinomycete isolate against fungal wheat pathogens, measured after 4 and 7 days incubation.
Very strong antagonism (+++),
Strong antagonism (++),
Moderate antagonism (+),
Weak antagonism (+/−),
No antagonism (−),
Not done (ND).

TABLE 14

| Endophyte | | Inhibition of F. graminearum on half |
|---|---|---|
| Isolate | Nearest 16S rDNA match | strength PDA |
| SE2 | S. triticum | 26.7% |
| EN27 | S. triticum | 60.0% |
| SE1 | S. triticum | 60.0% |
| EN28 | S. triticum | 53.3% |
| EN57 | S. triticum | 43.3% |
| EN35 | S. triticum | 50.0% |
| EN2 | Microbispora sp. | 43.3%^{a} |
| EN59 | S. galilaeus | 46.7% |
| EN43 | Micromonospora sp. | 6.7% |
| EN39 | S. galilaeus | 16.7% |

^{a}This isolate caused generally reduced vigour of the pathogen, including a reduction in aerial mycelium production and reduced growth even away from the actinomycete.

TABLE 15

| | % root | % shoot | % germ | |
|---|---|---|---|---|
| Isolate | inc. | inc. | inc. | Significant results |
| EN2 | −1 | −11 | 20 | |
| EN3 | 24 | 2 | 20 | root p < 0.10 |
| EN4 | 8 | 3 | 4 | |
| EN5 | 2 | 1 | 16 | root p < 0.05, germination |
| EN6 | 36 | 8 | 24 | p = 0.073 |
| EN7 | 19 | 8 | 12 | |
| EN9 | 17 | −1 | 20 | |
| EN10 | 25 | 6 | 16 | root p < 0.10 |
| EN16 | 24 | 9 | −4 | root p < 0.10 |
| EN17 | 9 | −2 | −4 | |
| EN19 | −6 | −6 | 24 | germination p = 0.034 |
| EN22 | 14 | 16 | 0 | |
| EN23 | 12 | −3 | 0 | |
| EN26 | −3 | 2 | 0 | |
| EN27 | 34 | 11 | −4 | root p < 0.05 |
| EN28 | −28 | −9 | −16 | root p < 0.05 |
| EN30 | 1 | −4 | 4 | |
| EN35 | −2 | 0 | −8 | |
| EN39 | −16 | 11 | −12 | |
| EN43 | −2 | −10 | 16 | |
| EN46 | −3 | 7 | −4 | |
| EN47 | −9 | −2 | −4 | |
| EN57 | 7 | 14 | −4 | shoot p < 0.01 |
| EN58 | 5 | 6 | −20 | shoot p < 0.10, germination |
| EN59 | −3 | 15 | −16 | p = 0.035 |
| EN60 | −2 | 3 | 0 | |
| SE1 | −25 | −23 | 0 | root and shoot p < 0.01 |
| SE2 | 29 | −11 | 20 | root p < 0.10 |

Red numbers indicate results significant at p<0.05, blue numbers indicate results where p<0.10.

TABLE 16
Statistical data (T-tests)

| | | |
|---|---|---|
| Isolate | % Change | plant part |

p < 0.05

| | | |
|---|---|---|
| EN6 | 36 | root |
| EN19 | 24 | germination |
| EN27 | 34 | root |
| EN28 | −28 | root |
| EN59 | −16 | germination |

0.05 < p < 0.10

| | | |
|---|---|---|
| EN3 | 24 | root |
| EN6 | 24 | germination |
| EN10 | 25 | root |
| EN16 | 24 | root |
| EN57 | 14 | shoot |
| EN59 | 15 | shoot |
| SE1 | −25 | root |
| | −23 | shoot |
| SE2 | 29 | root |

Those isolates shown in bold type are isolates that belong to Streptomyces triticum or Streptomyces triticum var. griseoviride.

TABLE 17
In planta biocontrol activity of endophytic actinobacteria
against Ggt8 in steamed soil.

| | | Ggt bio- | | | | Ggt bio- | |
|---|---|---|---|---|---|---|---|
| | | control^{a} | | | Isolate | control^{a} | |
| EN | Isolate | % | P | EN | sequence match | % | P |
| 2 | Microbispora sp. | 31 | 0.066 | 17 | S. triticum | 27 | 0.107 |
| 43 | Micromonospora | 25 | 0.044 | 19 | S. triticum | −4 | 0.722 |
| | sp. | | | | | | |
| 46 | Nocardioides | 25 | 0.064 | 22 | S. triticum | 41 | 0.028 |
| | albus | | | | | | |
| 47 | Nocardioides | 31 | 0.007 | 23 | S. triticum | 34 | 0.006 |
| | albus | | | | | | |
| 13 | Streptomyces sp. | −4 | 0.749 | 27 | S. triticum | 27 | 0.018 |
| 18 | Streptomyces sp. | −3 | 0.745 | 28 | S. triticum | 27 | 0.003 |
| 33 | Streptomyces sp. | 11 | 0.567 | 35 | S. triticum | 40 | 0.000 |
| 36 | Streptomyces sp. | 18 | 0.143 | 57 | S. triticum | 36 | 0.022 |
| 37 | Streptomyces sp. | −4 | 0.774 | 3 | S. galilaeus | 15 | 0.349 |
| 38 | Streptomyces sp. | 10 | 0.446 | 4 | S. galilaeus | 29 | 0.006 |
| 51 | Streptomyces sp. | 21 | 0.094 | 39 | S. galilaeus | 61 | 0.001 |
| 58 | Streptomyces sp. | −1 | 0.934 | 50 | S. galilaeus | 30 | 0.035 |
| 30 | S. argenteolus | 26 | 0.022 | 32 | S. neyagawensis | −6 | 0.606 |
| 54 | S. argenteolus | 4 | 0.743 | 52 | S. pseudovenezuelae | 19 | 0.142 |
| 60 | S. argenteolus | 37 | 0.002 | 53 | S. pseudovenezuelae | 26 | 0.099 |
| 8 | S. bottropensis | 11 | 0.458 | 61 | S. maritimus | 3 | 0.794 |
| 9 | S. bikiniensis | 17 | 0.068 | 26 | S. peruviensis | 13 | 0.310 |
| 5 | S. triticum | 0 | 0.977 | 20 | S. subrutilus | 13 | 0.307 |
| 16 | S. triticum | 35 | 0.054 | 34 | S. violarus | 7 | 0.463 |

^{a}Percentage of mean change in disease rating compared to the control. Negative values represent an increase in disease rating.

TABLE 18
Biocontrol of Ggt and Rhizoctonia solani naturally present
in field soil by endophytic actinobacteria that showed
significant activity in steamed soil

| | | Ggt bio- | | | | | |
|---|---|---|---|---|---|---|---|
| | | control^{a} | | Ggt bio- | | Rhizoctonia | |
| | | (steamed | | control^{a} | | biocontrol^{a} | |
| | | soil) | | (field soil) | | (field soil) | |
| EN | Isolate | % | P | % | P | % | P |
| 2 | Microbispora sp. | 31 | 0.066 | 53 | 0.001 | 39 | 0.10 |
| 43 | Micromonospora sp. | 25 | 0.044 | 22 | 0.233 | −89 | 0.00 |
| 46 | Nocardioides albus | 25 | 0.064 | 71 | 0.001 | 36 | 0.20 |
| 47 | Nocardioides albus | 31 | 0.007 | 43 | 0.071 | 19 | 0.49 |
| 51 | Streptomyces sp. | 21 | 0.094 | 32 | 0.126 | 11 | 0.67 |
| 30 | S. argenteolus | 26 | 0.022 | 41 | 0.020 | 35 | 0.15 |
| 60 | S. argenteolus | 37 | 0.002 | 54 | 0.002 | 55 | 0.02 |
| 9 | S. bikiniensis | 17 | 0.068 | 18 | 0.231 | 43 | 0.08 |
| 16 | S. triticum var. | 35 | 0.054 | 32 | 0.108 | 16 | 0.47 |
| | griseoviride | | | | | | |
| 22 | S. triticum | 41 | 0.028 | 20 | 0.241 | 51 | 0.06 |
| 23 | S. triticum | 34 | 0.006 | 14 | 0.363 | 44 | 0.09 |
| 27 | S. triticum | 27 | 0.018 | 40 | 0.013 | 35 | 0.15 |
| 28 | S. triticum | 27 | 0.003 | 20 | 0.403 | 49 | 0.11 |
| 35 | S. triticum | 40 | 0.000 | 41 | 0.042 | −20 | 0.44 |
| 57 | S. triticum | 36 | 0.022 | 19 | 0.295 | 44 | 0.06 |
| 4 | S. galilaeus | 29 | 0.006 | 11 | 0.541 | 16 | 0.55 |
| 39 | S. galilaeus | 61 | 0.001 | 22 | 0.239 | 29 | 0.30 |

^{a}Percentage of mean change in disease rating compared to the control. Negative values indicate an increase in disease rating.

TABLE 19

| | Barley shoot | Barley shoot | | |
|---|---|---|---|---|
| | dry wt % | length % | Barley Germ | Oat Shoot dry |
| Endophyte | change | change | % change | wt % change |
| EN2 | 15.94 | 15.7 | 10 | — |
| EN3 | 18.94 | 21.8 | 13.3 | 12.8 |
| EN6 | 17.3 | 22.1 | 0 | 9.5 |
| EN16 | — | 12.7 | 3.3 | — |
| EN27 | 15.3 | 27.8 | 16.7 | — |
| EN57 | 13.3 | 29.7 | 3.3 | 15.6 |
| EN60 | 19.3 | 21.8 | 10.0 | 6.5 |
| SE1 | 11.3 | 19.7 | 3.3 | 17 |
| SE2 | 16.3 | 22.6 | 6.7 | — |

TABLE 20

| | Wheat root length | Wheat shoot |
|---|---|---|
| Endophyte | % change | length % change |
| PM87 | 56 | 25 |
| PM185 | 41 | 23 |
| PM208 | — | 20 |
| PM330 | 65 | 24 |

Results of Field trials in the 2003 growing season

TABLE 21
Field Trial results showing increase in grain yield with actinomycete endophyte treatments

| | | | Bute | | Murray | | Pine Point | | | |
|---|---|---|---|---|---|---|---|---|---|---|
| | Bute | | Crown | | Bridge | | Take- | | Smoky Bay | |
| | Take-all² | | Rot³ | | Rhizoc¹ | | all/Growth² | | Rhizoc² | |
| Treatment | kg/ha | % of UT | kg/ha | % of UT | kg/ha | % of UT | kg/ha | % of UT | kg/ha | % of UT |
| EN27 low | 2182 | 99 | 1802 | 105 | 1517 | 91 | 2196 | 100 | 823 | 99.5 |
| EN27 high | 2253 | 102 | 1761 | 102 | 1829 | 109 | 2389* | 109 | 872 | 105 |
| EN16 low | 2285 | 103 | 1685 | 98 | 1828 | 109 | 2179 | 100 | 865 | 105 |
| En16 high | 2326* | 105 | 1726 | 100 | 1647 | 98 | 2174 | 99 | 859 | 104 |
| EN46 low | 2260 | 102 | 1953* | 114 | 2052 | 122 | 2157 | 99 | 906* | 110 |
| EN46 high | 2187 | 99 | nd | nd | nd | nd | 2009 | 92 | nd | nd |
| EN28 low | nd | nd | 1792 | 104 | 1438 | 86 | 2313 | 106 | 874 | 106 |
| EN28 high | nd | nd | 1701 | 99 | 1849* | 110 | 2232 | 102 | 825 | 100 |
| EN60 low | 2272 | 103 | nd | nd | nd | nd | nd | nd | nd | nd |
| EN60 high | 2259 | 102 | nd | nd | nd | nd | nd | nd | nd | nd |
| Jockey | 2359* | 107 | nd | nd | nd | nd | 2541* | 116 | nd | nd |
| Untreated | 2212 | 100 | 1720 | 100 | 1676 | 100 | 2188 | 100 | 827 | 100 |

*these grain yields were statistically higher than the “untreated”
% of UT = % of untreated
nd = not done
¹Barque barley
²Frame wheat
³durum wheat

TABLE 22
Field Trial results showing increase in grain yield with actinomycete endophyte treatments

| | Haslam | | Burra | | Esperance | | Tumby Bay | | Paskeville |
|---|---|---|---|---|---|---|---|---|---|
| | Rhizoc¹ | % of | Rhizoc² | % of | Rhizoc | % of | Growth² | % of | Pythium² |
| Treatment | kg/ha | UT | kg/ha | UT | kg/ha | UT | kg/ha | UT | kg/ha |
| EN27 low | 1813 | 95 | 1740 | 100 | nd | | 2394 | 98 | 2844 |
| EN27 high | 1756 | 92 | 1626 | 93 | 2136 | 110 | 2387 | 98 | 2783 |
| EN16 low | 1996 | 105 | 1614 | 93 | nd | | 2394 | 98 | 2744 |
| En16 high | 1882 | 99 | 1801 | 103 | nd | | 2481 | 101 | 2797 |
| EN46 low | 1854 | 98 | 1678 | 96 | nd | | 2383 | 97 | 2654 |
| EN46 high | nd | | 1755 | 101 | 2451 | 126 | 2533 | 104 | 2783 |
| EN28 low | 1944 | 102 | 1684 | 97 | nd | | 2484 | 102 | nd |
| EN28 high | 2015 | 106 | 1851 | 106 | nd | | 2373 | 97 | 2900 |
| EN60 low | nd | | nd | | nd | | nd | | nd |
| EN60 high | nd | | nd | | nd | | nd | | nd |
| EN23 low | nd | | nd | | nd | | nd | | 2720 |
| EN23 high | nd | | nd | | nd | | nd | | 2974 |
| Jockey | nd | | nd | | nd | | 2529 | 103 | nd |
| Untreated | 1899 | 100 | 1743 | 100 | 1948 | 100 | 2446 | 100 | 3115 |

| | | | | Marinna | | Tamworth | |
|---|---|---|---|---|---|---|---|
| | | Meningie | | NSW | | Crown | |
| | % of | Pythium² | % of | Pythium² | % of | Rot³ | % of |
| Treatment | UT | kg/ha | UT | kg/ha | UT | kg/ha | UT |
| EN27 low | 91 | 587 | 93 | 1616 | 93 | nd | |
| EN27 high | 89 | 507 | 80 | 1476 | 85 | 3410 | 101 |
| EN16 low | 88 | 396 | 63 | 1620 | 93 | nd | |
| En16 high | 90 | 600 | 95 | 1667 | 96 | nd | |
| EN46 low | 85 | 537 | 85 | 1531 | 88 | nd | |
| EN46 high | 89 | nd | | 1555 | 90 | 3290 | 98 |
| EN28 low | | nd | | nd | | nd | |
| EN28 high | 93 | 500 | 79 | 1567 | 90 | nd | |
| EN60 low | | nd | | nd | | nd | |
| EN60 high | | nd | | nd | | nd | |
| EN23 low | 87 | 602 | 95 | 1309 | 75 | nd | |
| EN23 high | 95 | 533 | 84 | 1705 | 98 | nd | |
| Jockey | | nd | | nd | | nd | |
| Untreated | 100 | 631 | 100 | 1736 | 100 | 3360 | 100 |

Explanation:
The treatment names eg EN27, EN46 etc are the identification numbers given to all the actinomycetes we have isolated. The actual number has no meaning, it is just the number we use to identify all the hundreds of different strains we have. “low and high” are the rates - we applied the spores at a low and a high rate.
“Untreated” is as it sounds.
% of UT = % of untreated;
nd = not done;
¹Barque barley;
²Frame wheat;
³Bellaroi durum wheat

### BIBLIOGRAPHY

Bonner et al., (1973) J. Mol. Biol. 81:123
Coombs, J. T. and Franco, C. M. M. (2003) Isolation and Identification of Actinobacteria from Surface-Sterilized Wheat Roots, Appl. Environ. Microbiol, 69:5603-5608.
Coombs, J. T. and Franco, C. M. M. (2003) Visualization of an endophytic Streptomyces species in wheat seed. Appl. Environ. Microbiol. 69(7):4260-4262.
Conn, V. M. and Franco, C. M. M. (2004). Analysis of the endophytic actinobacterial population in the roots of wheat (Triticum aestivum L.) by Terminal Restriction Fragment Length Polymorphism (T-RFLP) and sequencing of 16S rRNA clones. Applied and Environmental Microbiology 70:1787-1794.
Crawford, D. L., Lynch, J. M., Whipps, J. M., Ousley, M. a. (1993) Applied & Environmental Microbiology 59(11):3899-3905.
Flett, F. et al. (1997) FEMS Microbiol. Lett. 155:223-229.
Glickman, E. and Dessaux, Y. (1995) Applied and Environmental Microbiology 61:793-796.
Kieser, T. et al. (2000) Practical Streptomyces genetics. The John Innes Foundation, Norwich.
Mazodier, P. et al. (1989) J. Bacteriol. 171:3583-3585.
Roget, D. K., Coppi, J. A., Herdina, Gupta VVSR (1999) Proceedings of the 1^{st} Australiasian Soilborne Disease Symposium Magarey (eds).
Sun, J. (1999) Microbiol. 145:2221-2227.

1. A method of improving plant productivity said method comprising introducing into said plant or propagation material thereof:
(i) an effective number of endophytic actinomycetes or variants, mutants or homologues thereof, which actinomycetes facilitate induction of at least one characteristic related to improved productivity; and/or
(ii) an effective amount of one or more metabolites derived from the actinomycetes of (i) or derivative, homologue, analogue, chemical equivalent or mimetic thereof;
for a time and under conditions sufficient to induce, in the subject plant, said characteristic, and wherein said actinomycete is selected from:
(a) An actinomycete characterised either by a nucleotide sequence corresponding to the nucleotide sequence substantially as set forth in <400>1 or a nucleotide sequence capable of hybridising to <400>1 under low stringency conditions at 42° C. or a variant, mutant or homologue of said actinomycete.
(b) An actinomycete characterised either by a nucleotide sequence corresponding to the nucleotide sequence substantially as set forth in <400>2 or a nucleotide sequence capable of hybridising to <400>2 under low stringency conditions at 42° C. or a variant, mutant or homologue of said actinomycete.
(c) An actinomycete characterised either by a nucleotide sequence corresponding to the nucleotide sequence substantially as set forth in <400>7 or a nucleotide sequence capable of hybridising to <400>7 under low stringency conditions at 42° C. or a variant, mutant or homologue of said actinomycete.
(d) An actinomycete characterised either by a nucleotide sequence corresponding to the nucleotide sequence substantially as set forth in <400>10 or a nucleotide sequence capable of hybridising to <400>10 under low stringency conditions at 42° C. or a variant, mutant or homologue of said actinomycete.
(e) An actinomycete characterised either by a nucleotide sequence corresponding to the nucleotide sequence substantially as set forth in <400>12 or a nucleotide sequence capable of hybridising to <400>12 under low stringency conditions at 42° C. or a variant, mutant or homologue of said actinomycete.
(f) An actinomycete characterised either by a nucleotide sequence corresponding to the nucleotide sequence substantially as set forth in <400>13 or a nucleotide sequence capable of hybridising to <400>13 under low stringency conditions at 42° C. or a variant, mutant or homologue of said actinomycete.
(g) An actinomycete characterised either by a nucleotide sequence corresponding to the nucleotide sequence substantially as set forth in <400>16 or a nucleotide sequence capable of hybridising to <400>16 under low stringency conditions at 42° C. or a variant, mutant or homologue of said actinomycete.
(h) An actinomycete characterised either by a nucleotide sequence corresponding to the nucleotide sequence substantially as set forth in <400>18 or a nucleotide sequence capable of hybridising to <400>18 under low stringency conditions at 42° C. or a variant, mutant or homologue of said actinomycete
(i) An actinomycete characterised either by a nucleotide sequence corresponding to the nucleotide sequence substantially as set forth in <400>24 or a nucleotide sequence capable of hybridising to <400>24 under low stringency conditions at 42° C. or a variant, mutant or homologue of said actinomycete.

2. A method of improving plant productivity said method comprising introducing into said plant or propagation material thereof:
(i) an effective number of endophytic actinomycetes or variants, mutants or homologues thereof, which actinomycetes facilitate induction of at least one characteristic related to improved productivity; and/or
(ii) an effective amount of one or more metabolites derived from the actinomycetes of (i) or derivative, homologue, analogue, chemical equivalent or mimetic thereof;
for a time and under conditions sufficient to induce, in the subject plant, said characteristic, and wherein said actinomycete is selected from:
(a) An actinomycete characterised either by a nucleotide sequence corresponding to the nucleotide sequence substantially as set forth in <400>3 or a nucleotide sequence capable of hybridising to <400>3 under low stringency conditions at 42° C. or a variant, mutant or homologue of said actinomycete.
(b) An actinomycete characterised either by a nucleotide sequence corresponding to the nucleotide sequence substantially as set forth in <400>4 or a nucleotide sequence capable of hybridising to <400>4 under low stringency conditions at 42° C. or a variant, mutant or homologue of said actinomycete.
(c) An actinomycete characterised either by a nucleotide sequence corresponding to the nucleotide sequence substantially as set forth in <400>5 or a nucleotide sequence capable of hybridising to <400>5 under low stringency conditions at 42° C. or a variant, mutant or homologue of said actinomycete.
(d) An actinomycete characterised either by a nucleotide sequence corresponding to the nucleotide sequence substantially as set forth in <400>6 or a nucleotide sequence capable of hybridising to <400>6 under low stringency conditions at 42° C. or a variant, mutant or homologue of said actinomycete.
(e) An actinomycete characterised either by a nucleotide sequence corresponding to the nucleotide sequence substantially as set forth in <400>8 or a nucleotide sequence capable of hybridising to <400>8 under low stringency conditions at 42° C. or a variant, mutant or homologue of said actinomycete.
(f) An actinomycete characterised either by a nucleotide sequence corresponding to the nucleotide sequence substantially as set forth in <400>9 or a nucleotide sequence capable of hybridising to <400>9 under low stringency conditions at 42° C. or a variant, mutant or homologue of said actinomycete.
(g) An actinomycete characterised either by a nucleotide sequence corresponding to the nucleotide sequence substantially as set forth in <400>11 or a nucleotide sequence capable of hybridising to <400>11 under low stringency conditions at 42° C. or a variant, mutant or homologue of said actinomycete.
(h) An actinomycete characterised either by a nucleotide sequence corresponding to the nucleotide sequence substantially as set forth in <400>14 or a nucleotide sequence capable of hybridising to <400>14 under low stringency conditions at 42° C. or a variant, mutant or homologue of said actinomycete.
(i) An actinomycete characterised either by a nucleotide sequence corresponding to the nucleotide sequence substantially as set forth in <400>15 or a nucleotide sequence capable of hybridising to <400>15 under low stringency conditions at 42° C. or a variant, mutant or homologue of said actinomycete.
(j) An actinomycete characterised either by a nucleotide sequence corresponding to the nucleotide sequence substantially as set forth in <400>17 or a nucleotide sequence capable of hybridising to <400>17 under low stringency conditions at 42° C. or a variant, mutant or homologue of said actinomycete,
(k) An actinomycete characterised either by a nucleotide sequence corresponding to the nucleotide sequence substantially as set forth in <400>19 or a nucleotide sequence capable of hybridising to <400>19 under low stringency conditions at 42° C. or a variant, mutant or homologue of said actinomycete.
(l) An actinomycete characterised either by a nucleotide sequence corresponding to the nucleotide sequence substantially as set forth in <400>20 or a nucleotide sequence capable of hybridising to <400>20 under low stringency conditions at 42° C. or a variant, mutant or homologue of said actinomycete.
(m) An actinomycete characterised either by a nucleotide sequence corresponding to the nucleotide sequence substantially as set forth in <400>21 or a nucleotide sequence capable of hybridising to <400>21 under low stringency conditions at 42° C. or a variant, mutant or homologue of said actinomycete.
(n) An actinomycete characterised either by a nucleotide sequence corresponding to the nucleotide sequence substantially as set forth in <400>22 or a nucleotide sequence capable of hybridising to <400>22 under low stringency conditions at 42° C. or a variant, mutant or homologue of said actinomycete.
(o) An actinomycete characterised either by a nucleotide sequence corresponding to the nucleotide sequence substantially as set forth in <400>23 or a nucleotide sequence capable of hybridising to <400>23 under low stringency conditions at 42° C. or a variant, mutant or homologue of said actinomycete.
(p) An actinomycete characterised either by a nucleotide sequence corresponding to the nucleotide sequence substantially as set forth in <400>25 or a nucleotide sequence capable of hybridising to <400>25 under low stringency conditions at 42° C. or a variant, mutant or homologue of said actinomycete.
(q) An actinomycete characterised either by a nucleotide sequence corresponding to the nucleotide sequence substantially as set forth in <400>26 or a nucleotide sequence capable of hybridising to <400>26 under low stringency conditions at 42° C. or a variant, mutant or homologue of said actinomycete.
(r) An actinomycete characterised either by a nucleotide sequence corresponding to the nucleotide sequence substantially as set forth in <400>27 or a nucleotide sequence capable of hybridising to <400>27 under low stringency conditions at 42° C. or a variant, mutant or homologue of said actinomycete.
(s) An actinomycete characterised either by a nucleotide sequence corresponding to the nucleotide sequence substantially as set forth in <400>28 or a nucleotide sequence capable of hybridising to <400>28 under low stringency conditions at 42° C. or a variant, mutant or homologue of said actinomycete.
(t) An actinomycete characterised either by a nucleotide sequence corresponding to the nucleotide sequence substantially as set forth in <400>29 or a nucleotide sequence capable of hybridising to <400>29 under low stringency conditions at 42° C. or a variant, mutant or homologue of said actinomycete
(u) An actinomycete characterised either by a nucleotide sequence corresponding to the nucleotide sequence substantially as set forth in <400>30 or a nucleotide sequence capable of hybridising to <400>30 under low stringency conditions at 42° C. or a variant, mutant or homologue of said actinomycete.

3. The method according to claim 1 or 2, wherein said actinomycete is characterised by a nucleotide sequence which has at least about 45% similarity to all or part of the nucleotide sequence indicated by the nucleotide sequence identification number.

4. The method according to claim 3 wherein said similarity is 50%, preferably 55%, more preferably 60%, still more preferably 65%, even more preferably 70% and most preferably 80%.

5. The method according to claim 3 wherein said actinomycete is selected from:

| | | | |
|---|---|---|---|
| (a) EN2 | (b) EN3 | (c) EN16 | (d) EN23 |
| (e) EN27 | (f) EN28 | (g) EN46 | (h) EN60 |
| (i) PM87. | | | |

6. The method according to claim 3 wherein said actinomycete is selected from:

| | | | |
|---|---|---|---|
| (a) EN5 | (b) EN6 | (c) EN7 | (d) EN9 |
| (e) EN17 | (f) EN19 | (g) EN26 | (h) EN35 |
| (i) EN39 | (j) EN57 | (k) SE1 | (1) SE2 |
| (m) PM36 | (n) PM40 | (o) PM41 | (p) PM171 |
| (q) PM185 | (r) PM208 | (s) PM228 | (t) PM252 |
| (u) PM342 | | | |

7. The method according to any one of claims 1-6 wherein said metabolite is auxin, gibberellin, cytokinin, indole acetic acid, kinetin or signal molecule able to induce resistance in plants.

8. The method according to any one of claims 1-6 wherein said metabolite is an antibiotic compound.

9. The method according to any one of claims 1-7 wherein said productivity is growth promotion characteristics and/or bio-control characteristics.

10. The method according to claim 9 wherein said growth promotion characteristic is one or more of rate of growth, plant vigour, yield of flower/fruit/grain, vitality of crop or improved seed germination.

11. The method according to claim 9 wherein said bio-control characteristic is a decrease in susceptibility to pathogen infection or an increase in the clearance efficiency of infection.

12. The method according to any one of claims 1-11 wherein said plant is a cereal crop.

13. The method according to claim 12 wherein said cereal crop is a wheat, barley, maize, triticale, rye, oats, canary, sorghum, millet or rice.

14. The method according to claim 11 wherein said bio-control activity is bio-control in relation to Gaeumannomyces graminis var. tritici, Pythium ssp., Rhizoctonia solani, Fusarium sp., insect or nematode.

15. The method according to claim 14 wherein said insect is an aphid.

16. The method according to claim 14 or 15 wherein said plant is a cereal plant.

17. The method according to any one of claims 14-16 wherein said actinomycete is selected from EN2, EN3, EN16, EN23, EN27, EN28, EN46, EN60 or PM87.

18. The method according to any one of claims 14-16 wherein said actinomycete is selected from EN9, EN17, EN19, EN26, EN35, EN39, EN57 or SE1.

19. The method according to claim 9 wherein said actinomycete is selected from EN2, EN3, EN16, EN27, EN60 or PM87 and said improved productivity is improved plant growth promotion.

20. The method according to claim 9 wherein said actinomycete is selected from EN6, EN9, EN57, SE1, SE3, PM185 or PM208 and said improved productivity is improved plant growth promotion.

21. The method according to claim 19 or 20 wherein said growth promotion is germination promotion.

22. The method according to claim 19, 20 or 21 wherein said plant is a cereal plant.

23. The method according to claim 9 wherein said actinomycete is selected from EN2, EN3, EN16, EN23, EN27, EN28, EN46, EN60 or PM87 and said improved productivity is improved bio-control activity and improved plant growth promotion.

24. The method according to claim 9 wherein said actinomycete is selected from EN9, EN35, EN57, SE1 or SE1 and said improved productivity is improved bio-control activity and improved plant growth promotion.

25. The method according to claim 23 or 24 wherein said plant is a cereal plant.

26. The method according to claim 9 wherein said actinomycete is selected from EN2, EN3, EN16, EN23, EN27, EN28, EN46 or PM87 and said improved productivity is improved bio-control activity.

27. The method according to claim 9 wherein said actinomycete is selected from EN5, EN17, EN19 or EN35 and said improved productivity is improved bio-control activity.

28. The method according to claim 26 or 27 wherein said bio-control activity is bio-control in relation to aphids.

29. The method according to claim 26, 27 or 28 wherein said plant is a cereal plant.

30. The method according to any one of claims 16, 22, 25 or 29 wherein said cereal plant is wheat, barley, maize, rye, triticale, oats, canary seed, sorghum, millet or rice.

31. A method of improving plant productivity said method comprising introducing into said plant or propagation material thereof:
(i) an effective number of at least teo endophytic actinomycete strains or variants, mutants or homologues thereof, which actinomycetes facilitate induction of at least one characteristic related to improved productivity; and/or
(ii) an effective amount of one or more metabolites derived from the actinomycetes of (i) or derivative, homologue, analogue, chemical equivalent or mimetic thereof;
for a time and under conditions sufficient to induce, in the subject plant, said characteristic, and wherein said at least two endophytic actinomycete strains are selected from:
(a) EN2, EN9 and EN23
(b) EN9, EN27 and EN28
(c) EN39 and EN46.

32. A cereal plant-derived endophytic actinomycete or variants, mutants or homologues thereof or metabolites derived therefrom or derivatives, homologues, analogues, chemical equivalents or mimetics thereof for use in the method of any one of claims 1-31 wherein said actinomycete is selected from:
(a) An actinomycete characterised either by a nucleotide sequence corresponding to the nucleotide sequence substantially as set forth in <400>1 or a nucleotide sequence capable of hybridising to <400>1 under low stringency conditions at 42° C. or a variant, mutant or homologue of said actinomycete.
(b) An actinomycete characterised either by a nucleotide sequence corresponding to the nucleotide sequence substantially as set forth in <400>2 or a nucleotide sequence capable of hybridising to <400>2 under low stringency conditions at 42° C. or a variant, mutant or homologue of said actinomycete.
(c) An actinomycete characterised either by a nucleotide sequence corresponding to the nucleotide sequence substantially as set forth in <400>7 or a nucleotide sequence capable of hybridising to <400>7 under low stringency conditions at 42° C. or a variant, mutant or homologue of said actinomycete.
(d) An actinomycete characterised either by a nucleotide sequence corresponding to the nucleotide sequence substantially as set forth in <400>10 or a nucleotide sequence capable of hybridising to <400>10 under low stringency conditions at 42° C. or a variant, mutant or homologue of said actinomycete.
(e) An actinomycete characterised either by a nucleotide sequence corresponding to the nucleotide sequence substantially as set forth in <400>12 or a nucleotide sequence capable of hybridising to <400>12 under low stringency conditions at 42° C. or a variant, mutant or homologue of said actinomycete.
(f) An actinomycete characterised either by a nucleotide sequence corresponding to the nucleotide sequence substantially as set forth in <400>13 or a nucleotide sequence capable of hybridising to <400>13 under low stringency conditions at 42° C. or a variant, mutant or homologue of said actinomycete.
(g) An actinomycete characterised either by a nucleotide sequence corresponding to the nucleotide sequence substantially as set forth in <400>16 or a nucleotide sequence capable of hybridising to <400>16 under low stringency conditions at 42° C. or a variant, mutant or homologue of said actinomycete.
(h) An actinomycete characterised either by a nucleotide sequence corresponding to the nucleotide sequence substantially as set forth in <400>18 or a nucleotide sequence capable of hybridising to <400>18 under low stringency conditions at 42° C. or a variant, mutant or homologue of said actinomycete
(i) An actinomycete characterised either by a nucleotide sequence corresponding to the nucleotide sequence substantially as set forth in <400>24 or a nucleotide sequence capable of hybridising to <400>24 under low stringency conditions at 42° C. or a variant, mutant or homologue of said actinomycete.

33. A cereal plant-derived endophytic actinomycete or variants, mutants or homologues thereof or metabolites derived therefrom or derivatives, homologues, analogues, chemical equivalents or mimetics thereof for use in the method of any one of claims 1-31 wherein said actinomycete is selected from:
(a) An actinomycete characterised either by a nucleotide sequence corresponding to the nucleotide sequence substantially as set forth in <400>3 or a nucleotide sequence capable of hybridising to <400>3 under low stringency conditions at 42° C. or a variant, mutant or homologue of said actinomycete.
(b) An actinomycete characterised either by a nucleotide sequence corresponding to the nucleotide sequence substantially as set forth in <400>4 or a nucleotide sequence capable of hybridising to <400>4 under low stringency conditions at 42° C. or a variant, mutant or homologue of said actinomycete.
(c) An actinomycete characterised either by a nucleotide sequence corresponding to the nucleotide sequence substantially as set forth in <400>5 or a nucleotide sequence capable of hybridising to <400>5 under low stringency conditions at 42° C. or a variant, mutant or homologue of said actinomycete.
(d) An actinomycete characterised either by a nucleotide sequence corresponding to the nucleotide sequence substantially as set forth in <400>6 or a nucleotide sequence capable of hybridising to <400>6 under low stringency conditions at 42° C. or a variant, mutant or homologue of said actinomycete.
(e) An actinomycete characterised either by a nucleotide sequence corresponding to the nucleotide sequence substantially as set forth in <400>8 or a nucleotide sequence capable of hybridising to <400>8 under low stringency conditions at 42° C. or a variant, mutant or homologue of said actinomycete.
(f) An actinomycete characterised either by a nucleotide sequence corresponding to the nucleotide sequence substantially as set forth in <400>9 or a nucleotide sequence capable of hybridising to <400>9 under low stringency conditions at 42° C. or a variant, mutant or homologue of said actinomycete.
(g) An actinomycete characterised either by a nucleotide sequence corresponding to the nucleotide sequence substantially as set forth in <400>11 or a nucleotide sequence capable of hybridising to <400>11 under low stringency conditions at 42° C. or a variant, mutant or homologue of said actinomycete.
(h) An actinomycete characterised either by a nucleotide sequence corresponding to the nucleotide sequence substantially as set forth in <400>14 or a nucleotide sequence capable of hybridising to <400>14 under low stringency conditions at 42° C. or a variant, mutant or homologue of said actinomycete.
(i) An actinomycete characterised either by a nucleotide sequence corresponding to the nucleotide sequence substantially as set forth in <400>15 or a nucleotide sequence capable of hybridising to <400>15 under low stringency conditions at 42° C. or a variant, mutant or homologue of said actinomycete.
(j) An actinomycete characterised either by a nucleotide sequence corresponding to the nucleotide sequence substantially as set forth in <400>17 or a nucleotide sequence capable of hybridising to <400>17 under low stringency conditions at 42° C. or a variant, mutant or homologue of said actinomycete.
(k) An actinomycete characterised either by a nucleotide sequence corresponding to the nucleotide sequence substantially as set forth in <400>19 or a nucleotide sequence capable of hybridising to <400>19 under low stringency conditions at 42° C. or a variant, mutant or homologue of said actinomycete.
(l) An actinomycete characterised either by a nucleotide sequence corresponding to the nucleotide sequence substantially as set forth in <400>20 or a nucleotide sequence capable of hybridising to <400>20 under low stringency conditions at 42° C. or a variant, mutant or homologue of said actinomycete.
(m) An actinomycete characterised either by a nucleotide sequence corresponding to the nucleotide sequence substantially as set forth in <400>21 or a nucleotide sequence capable of hybridising to <400>21 under low stringency conditions at 42° C. or a variant, mutant or homologue of said actinomycete.
(n) An actinomycete characterised either by a nucleotide sequence corresponding to the nucleotide sequence substantially as set forth in <400>22 or a nucleotide sequence capable of hybridising to <400>22 under low stringency conditions at 42° C. or a variant, mutant or homologue of said actinomycete.
(O) An actinomycete characterised either by a nucleotide sequence corresponding to the nucleotide sequence substantially as set forth in <400>23 or a nucleotide sequence capable of hybridising to <400>23 under low stringency conditions at 42° C. or a variant, mutant or homologue of said actinomycete.
(p) An actinomycete characterised either by a nucleotide sequence corresponding to the nucleotide sequence substantially as set forth in <400>25 or a nucleotide sequence capable of hybridising to <400>25 under low stringency conditions at 42° C. or a variant, mutant or homologue of said actinomycete.
(q) An actinomycete characterised either by a nucleotide sequence corresponding to the nucleotide sequence substantially as set forth in <400>26 or a nucleotide sequence capable of hybridising to <400>26 under low stringency conditions at 42° C. or a variant, mutant or homologue of said actinomycete.
(r) An actinomycete characterised either by a nucleotide sequence corresponding to the nucleotide sequence substantially as set forth in <400>27 or a nucleotide sequence capable of hybridising to <400>27 under low stringency conditions at 42° C. or a variant, mutant or homologue of said actinomycete.
(s) An actinomycete characterised either by a nucleotide sequence corresponding to the nucleotide sequence substantially as set forth in <400>28 or a nucleotide sequence capable of hybridising to <400>28 under low stringency conditions at 42° C. or a variant, mutant or homologue of said actinomycete.
(t) An actinomycete characterised either by a nucleotide sequence corresponding to the nucleotide sequence substantially as set forth in <400>29 or a nucleotide sequence capable of hybridising to <400>29 under low stringency conditions at 42° C. or a variant, mutant or homologue of said actinomycete
(u) An actinomycete characterised either by a nucleotide sequence corresponding to the nucleotide sequence substantially as set forth in <400>30 or a nucleotide sequence capable of hybridising to <400>30 under low stringency conditions at 42° C. or a variant, mutant or homologue of said actinomycete.

34. An agricultural composition comprising an endophytic actinomycete or metabolite derived therefrom together with one or more agriculturally acceptable carriers and/or diluents wherein said actinomycete is selected from:
(a) An actinomycete characterised either by a nucleotide sequence corresponding to the nucleotide sequence substantially as set forth in <400>1 or a nucleotide sequence capable of hybridising to <400>1 under low stringency conditions at 42° C. or a variant, mutant or homologue of said actinomycete.
(b) An actinomycete characterised either by a nucleotide sequence corresponding to the nucleotide sequence substantially as set forth in <400>2 or a nucleotide sequence capable of hybridising to <400>2 under low stringency conditions at 42° C. or a variant, mutant or homologue of said actinomycete.
(c) An actinomycete characterised either by a nucleotide sequence corresponding to the nucleotide sequence substantially as set forth in <400>7 or a nucleotide sequence capable of hybridising to <400>7 under low stringency conditions at 42° C. or a variant, mutant or homologue of said actinomycete.
(d) An actinomycete characterised either by a nucleotide sequence corresponding to the nucleotide sequence substantially as set forth in <400>10 or a nucleotide sequence capable of hybridising to <400>10 under low stringency conditions at 42° C. or a variant, mutant or homologue of said actinomycete.
(e) An actinomycete characterised either by a nucleotide sequence corresponding to the nucleotide sequence substantially as set forth in <400>12 or a nucleotide sequence capable of hybridising to <400>12 under low stringency conditions at 42° C. or a variant, mutant or homologue of said actinomycete.
(f) An actinomycete characterised either by a nucleotide sequence corresponding to the nucleotide sequence substantially as set forth in <400>13 or a nucleotide sequence capable of hybridising to <400>13 under low stringency conditions at 42° C. or a variant, mutant or homologue of said actinomycete.
(g) An actinomycete characterised either by a nucleotide sequence corresponding to the nucleotide sequence substantially as set forth in <400>16 or a nucleotide sequence capable of hybridising to <400>16 under low stringency conditions at 42° C. or a variant, mutant or homologue of said actinomycete.
(h) An actinomycete characterised either by a nucleotide sequence corresponding to the nucleotide sequence substantially as set forth in <400>18 or a nucleotide sequence capable of hybridising to <400>18 under low stringency conditions at 42° C. or a variant, mutant or homologue of said actinomycete
(i) An actinomycete characterised either by a nucleotide sequence corresponding to the nucleotide sequence substantially as set forth in <400>24 or a nucleotide sequence capable of hybridising to <400>24 under low stringency conditions at 42° C. or a variant, mutant or homologue of said actinomycete.

35. An agricultural composition comprising an endophytic actinomycete or metabolite derived therefrom together with one or more agriculturally acceptable carriers and/or diluents wherein said actinomycete is selected from:
(a) An actinomycete characterised either by a nucleotide sequence corresponding to the nucleotide sequence substantially as set forth in <400>3 or a nucleotide sequence capable of hybridising to <400>3 under low stringency conditions at 42° C. or a variant, mutant or homologue of said actinomycete.
(b) An actinomycete characterised either by a nucleotide sequence corresponding to the nucleotide sequence substantially as set forth in <400>4 or a nucleotide sequence capable of hybridising to <400>4 under low stringency conditions at 42° C. or a variant, mutant or homologue of said actinomycete.
(c) An actinomycete characterised either by a nucleotide sequence corresponding to the nucleotide sequence substantially as set forth in <400>5 or a nucleotide sequence capable of hybridising to <400>5 under low stringency conditions at 42° C. or a variant, mutant or homologue of said actinomycete.
(d) An actinomycete characterised either by a nucleotide sequence corresponding to the nucleotide sequence substantially as set forth in <400>6 or a nucleotide sequence capable of hybridising to <400>6 under low stringency conditions at 42° C. or a variant, mutant or homologue of said actinomycete.
(e) An actinomycete characterised either by a nucleotide sequence corresponding to the nucleotide sequence substantially as set forth in <400>8 or a nucleotide sequence capable of hybridising to <400>8 under low stringency conditions at 42° C. or a variant, mutant or homologue of said actinomycete.
(f) An actinomycete characterised either by a nucleotide sequence corresponding to the nucleotide sequence substantially as set forth in <400>9 or a nucleotide sequence capable of hybridising to <400>9 under low stringency conditions at 42° C. or a variant, mutant or homologue of said actinomycete.
(g) An actinomycete characterised either by a nucleotide sequence corresponding to the nucleotide sequence substantially as set forth in <400>11 or a nucleotide sequence capable of hybridising to <400>11 under low stringency conditions at 42° C. or a variant, mutant or homologue of said actinomycete.
(h) An actinomycete characterised either by a nucleotide sequence corresponding to the nucleotide sequence substantially as set forth in <400>14 or a nucleotide sequence capable of hybridising to <400>14 under low stringency conditions at 42° C. or a variant, mutant or homologue of said actinomycete.
(i) An actinomycete characterised either by a nucleotide sequence corresponding to the nucleotide sequence substantially as set forth in <400>15 or a nucleotide sequence capable of hybridising to <400>15 under low stringency conditions at 42° C. or a variant, mutant or homologue of said actinomycete.
(j) An actinomycete characterised either by a nucleotide sequence corresponding to the nucleotide sequence substantially as set forth in <400>17 or a nucleotide sequence capable of hybridising to <400>17 under low stringency conditions at 42° C. or a variant, mutant or homologue of said actinomycete.
(k) An actinomycete characterised either by a nucleotide sequence corresponding to the nucleotide sequence substantially as set forth in <400>19 or a nucleotide sequence capable of hybridising to <400>19 under low stringency conditions at 42° C. or a variant, mutant or homologue of said actinomycete.
(l) An actinomycete characterised either by a nucleotide sequence corresponding to the nucleotide sequence substantially as set forth in <400>20 or a nucleotide sequence capable of hybridising to <400>20 under low stringency conditions at 42° C. or a variant, mutant or homologue of said actinomycete.
(m) An actinomycete characterised either by a nucleotide sequence corresponding to the nucleotide sequence substantially as set forth in <400>21 or a nucleotide sequence capable of hybridising to <400>21 under low stringency conditions at 42° C. or a variant, mutant or homologue of said actinomycete.
(n) An actinomycete characterised either by a nucleotide sequence corresponding to the nucleotide sequence substantially as set forth in <400>22 or a nucleotide sequence capable of hybridising to <400>22 under low stringency conditions at 42° C. or a variant, mutant or homologue of said actinomycete.
(o) An actinomycete characterised either by a nucleotide sequence corresponding to the nucleotide sequence substantially as set forth in <400>23 or a nucleotide sequence capable of hybridising to <400>23 under low stringency conditions at 42° C. or a variant, mutant or homologue of said actinomycete.
(p) An actinomycete characterised either by a nucleotide sequence corresponding to the nucleotide sequence substantially as set forth in <400>25 or a nucleotide sequence capable of hybridising to <400>25 under low stringency conditions at 42° C. or a variant, mutant or homologue of said actinomycete.
(q) An actinomycete characterised either by a nucleotide sequence corresponding to the nucleotide sequence substantially as set forth in <400>26 or a nucleotide sequence capable of hybridising to <400>26 under low stringency conditions at 42° C. or a variant, mutant or homologue of said actinomycete.
(r) An actinomycete characterised either by a nucleotide sequence corresponding to the nucleotide sequence substantially as set forth in <400>27 or a nucleotide sequence capable of hybridising to <400>27 under low stringency conditions at 42° C. or a variant, mutant or homologue of said actinomycete.
(s) An actinomycete characterised either by a nucleotide sequence corresponding to the nucleotide sequence substantially as set forth in <400>28 or a nucleotide sequence capable of hybridising to <400>28 under low stringency conditions at 42° C. or a variant, mutant or homologue of said actinomycete.
(t) An actinomycete characterised either by a nucleotide sequence corresponding to the nucleotide sequence substantially as set forth in <400>29 or a nucleotide sequence capable of hybridising to <400>29 under low stringency conditions at 42° C. or a variant, mutant or homologue of said actinomycete
(u) An actinomycete characterised either by a nucleotide sequence corresponding to the nucleotide sequence substantially as set forth in <400>30 or a nucleotide sequence capable of hybridising to <400>30 under low stringency conditions at 42° C. or a variant, mutant or homologue of said actinomycete.

36. A novel, isolated endophytic actinomycete or variant, mutant or homologue thereof wherein said actinomycete is selected from:
(a) An actinomycete characterised either by a nucleotide sequence corresponding to the nucleotide sequence substantially as set forth in <400>1 or a nucleotide sequence capable of hybridising to <400>1 under low stringency conditions at 42° C. or a variant, mutant or homologue of said actinomycete.
(b) The actinomycete of (a) wherein said actinomycete corresponds to EN2 (AGAL Deposit No. NMO3/35895).
(c) An actinomycete characterised either by a nucleotide sequence corresponding to the nucleotide sequence substantially as set forth in <400>2 or a nucleotide sequence capable of hybridising to <400>2 under low stringency conditions at 42° C. or a variant, mutant or homologue of said actinomycete.
(d) The actinomycete of (c) wherein said actinomycete corresponds to EN3 (AGAL Deposit No. NM03/36501).
(e) An actinomycete characterised either by a nucleotide sequence corresponding to the nucleotide sequence substantially as set forth in <400>7 or a nucleotide sequence capable of hybridising to <400>7 under low stringency conditions at 42° C. or a variant, mutant or homologue of said actinomycete.
(f) The actinomycete of (e) wherein said aci corresponds to EN16 (AGAL Deposit No. NM03/35605).
(g) An actinomycete characterised either by a nucleotide sequence corresponding to the nucleotide sequence substantially as set forth in <400>10 or a nucleotide sequence capable of hybridising to <400>10 under low stringency conditions at 42° C. or a variant, mutant or homologue of said actinomycete.
(h) The actinomycete of (g) wherein said actinomycete corresponds to EN23 (AGAL Deposit No. NM03/35605).
(i) An actinomycete characterised either by a nucleotide sequence corresponding to the nucleotide sequence substantially as set forth in <400>12 or a nucleotide sequence capable of hybridising to <400>12 under low stringency conditions at 42° C. or a variant, mutant or homologue of said actinomycete.
(j) The actinomycete of (i) wherein said actinomycete corresponds to EN27 (AGAL Deposit No. NM03/35606).
(k) An actinomycete characterised either by a nucleotide sequence corresponding to the nucleotide sequence substantially as set forth in <400>13 or a nucleotide sequence capable of hybridising to <400>13 under low stringency conditions at 42° C. or a variant, mutant or homologue of said actinomycete.
(l) The actinomycete of (i) wherein said actinomycete corresponds to EN28 (AGAL Deposit No. NM03/35607).
(m) An actinomycete characterised either by a nucleotide sequence corresponding to the nucleotide sequence substantially as set forth in <400>16 or a nucleotide sequence capable of hybridising to <400>16 under low stringency conditions at 42° C. or a variant, mutant or homologue of said actinomycete.
(n) The actinomycete of (m) wherein said actinomycete corresponds to EN46 (AGAL Deposit No. NM03/34609).
(o) An actinomycete characterised either by a nucleotide sequence corresponding to the nucleotide sequence substantially as set forth in <400>18 or a nucleotide sequence capable of hybridising to <400>18 under low stringency conditions at 42° C. or a variant, mutant or homologue of said actinomycete.
(p) The actinomycete of (o) wherein said actinomycete corresponds to EN60 (AGAL Deposit No. NM03/35896).
(q) An actinomycete characterised either by a nucleotide sequence corresponding to the nucleotide sequence substantially as set forth in <400>24 or a nucleotide sequence capable of hybridising to <400>24 under low stringency conditions at 42° C. or a variant, mutant or homologue of said actinomycete.
(r) The actinomycete of (q) wherein said actinomycete corresponds to PM87 (AGAL Deposit No. NM03/35608).

37. A novel, isolated endophytic actinomycete or variant, mutant or homologue thereof wherein said actinomycete is selected from:
(a) An actinomycete characterised either by a nucleotide sequence corresponding to the nucleotide sequence substantially as set forth in <400>3 or a nucleotide sequence capable of hybridising to <400>3 under low stringency conditions at 42° C. or a variant, mutant or homologue of said actinomycete.
(b) The actinomycete according to (a) wherein said actinomycete corresponds to EN5.
(c) An actinomycete characterised either by a nucleotide sequence corresponding to the nucleotide sequence substantially as set forth in <400>4 or a nucleotide sequence capable of hybridising to <400>4 under low stringency conditions at 42° C. or a variant, mutant or homologue of said actinomycete.
(d) The actinomycete of (c) wherein said actinomycete corresponds to EN6.
(e) An actinomycete characterised either by a nucleotide sequence corresponding to the nucleotide sequence substantially as set forth in <400>5 or a nucleotide sequence capable of hybridising to <400>5 under low stringency conditions at 42° C. or a variant, mutant or homologue of said actinomycete.
(f) The actinomycete of (e) wherein said actinomycete corresponds to EN7.
(g) An actinomycete characterised either by a nucleotide sequence corresponding to the nucleotide sequence substantially as set forth in <400>6 or a nucleotide sequence capable of hybridising to <400>6 under low stringency conditions at 42° C. or a variant, mutant or homologue of said actinomycete.
(1) The actinomycete of (g) wherein said actinomycete corresponds to EN9.
(i) The actinomycete of (h) wherein said actinomycete is characterised either by a nucleotide sequence corresponding to the nucleotide sequence substantially as set forth in <400>8 or a nucleotide sequence capable of hybridising to <400>8 under low stringency conditions at 42° C. or a variant, mutant or homologue of said actinomycete.
(t) The actinomycete of (i) wherein said actinomycete corresponds to EN17.
(k) An actinomycete characterised either by a nucleotide sequence corresponding to the nucleotide sequence substantially as set forth in <400>9 or a nucleotide sequence capable of hybridising to <400>9 under low stringency conditions at 42° C. or a variant, mutant or homologue of said actinomycete.
(l) The actinomycete of (k) wherein said actinomycete corresponds to EN19.
(m) An actinomycete characterised either by a nucleotide sequence corresponding to the nucleotide sequence substantially as set forth in <400>11 or a nucleotide sequence capable of hybridising to <400>11 under low stringency conditions at 42° C. or a variant, mutant or homologue of said actinomycete.
(n) The actinomycete of (m) wherein said actinomycete corresponds to EN26.
(o) An actinomycete characterised either by a nucleotide sequence corresponding to the nucleotide sequence substantially as set forth in <400>14 or a nucleotide sequence capable of hybridising to <400>14 under low stringency conditions at 42° C. or a variant, mutant or homologue of said actinomycete.
(p) The actinomycete of (o) wherein said subject actinomycete corresponds to EN35.
(q) An actinomycete characterised either by a nucleotide sequence corresponding to the nucleotide sequence substantially as set forth in <400>15 or a nucleotide sequence capable of hybridising to <400>15 under low stringency conditions at 42° C. or a variant, mutant or homologue of said actinomycete.
(r) The actinomycete of (q) wherein said actinomycete corresponds to EN39.
(s) An actinomycete characterised either by a nucleotide sequence corresponding to the nucleotide sequence substantially as set forth in <400>17 or a nucleotide sequence capable of hybridising to <400>17 under low stringency conditions at 42° C. or a variant, mutant or homologue of said actinomycete.
(t) The actinomycete of (s) wherein said actinomycete corresponds to EN57.
(u) An actinomycete characterised either by a nucleotide sequence corresponding to the nucleotide sequence substantially as set forth in <400>19 or a nucleotide sequence capable of hybridising to <400>19 under low stringency conditions at 42° C. or a variant, mutant or homologue of said actinomycete.
(v) The actinomycete of (u) wherein said actinomycete corresponds to SE1.
(w) An actinomycete characterised either by a nucleotide sequence corresponding to the nucleotide sequence substantially as set forth in <400>20 or a nucleotide sequence capable of hybridising to <400>20 under low stringency conditions at 42° C. or a variant, mutant or homologue of said actinomycete.
(x) The actinomycete of (w) wherein said actinomycete corresponds to SE2.
(y) An actinomycete characterised either by a nucleotide sequence corresponding to the nucleotide sequence substantially as set forth in <400>21 or a nucleotide sequence capable of hybridising to <400>21 under low stringency conditions at 42° C. or a variant, mutant or homologue of said actinomycete.
(z) The actinomycete of (y) wherein said actinomycete corresponds to PM36.
(aa) An actinomycete characterised either by a nucleotide sequence corresponding to the nucleotide sequence substantially as set forth in <400>22 or a nucleotide sequence capable of hybridising to <400>22 under low stringency conditions at 42° C. or a variant, mutant or homologue of said actinomycete.
(ab) The actinomycete of (aa) wherein said actinomycete corresponds to PM40.
(ac) An actinomycete characterised either by a nucleotide sequence corresponding to the nucleotide sequence substantially as set forth in <400>23 or a nucleotide sequence capable of hybridising to <400>23 under low stringency conditions at 42° C. or a variant, mutant or homologue of said actinomycete.
(ad) The actinomycete of (ac) wherein said actinomycete corresponds to PM41.
(ae) An actinomycete characterised either by a nucleotide sequence corresponding to the nucleotide sequence substantially as set forth in <400>25 or a nucleotide sequence capable of hybridising to <400>25 under low stringency conditions at 42° C. or a variant, mutant or homologue of said actinomycete.
(af) The actinomycete of (ae) wherein said actinomycete corresponds to PM171.
(ag) An actinomycete characterised either by a nucleotide sequence corresponding to the nucleotide sequence substantially as set forth in <400>26 or a nucleotide sequence capable of hybridising to <400>26 under low stringency conditions at 42° C. or a variant, mutant or homologue of said actinomycete.
(ah) The actinomycete of (ag) wherein said actinomycete corresponds to PM185.
(ai) An actinomycete characterised either by a nucleotide sequence corresponding to the nucleotide sequence substantially as set forth in <400>27 or a nucleotide sequence capable of hybridising to <400>27 under low stringency conditions at 42° C. or a variant, mutant or homologue of said actinomycete.
(aj) The actinomycete of (ai) wherein said actinomycete corresponds to PM208.
(ak) An actinomycete characterised either by a nucleotide sequence corresponding to the nucleotide sequence substantially as set forth in <400>28 or a nucleotide sequence capable of hybridising to <400>28 under low stringency conditions at 42° C. or a variant, mutant or homologue of said actinomycete.
(al) The actinomycete of (ak) wherein said actinomycete corresponds to PM228.
(am) An actinomycete characterised either by a nucleotide sequence corresponding to the nucleotide sequence substantially as set forth in <400>29 or a nucleotide sequence capable of hybridising to <400>29 under low stringency conditions at 42° C. or a variant, mutant or homologue of said actinomycete.
(an) The actinomycete of (am) wherein said actinomycete corresponds to PM252.
(ao) An actinomycete characterised either by a nucleotide sequence corresponding to the nucleotide sequence substantially as set forth in <400>30 or a nucleotide sequence capable of hybridising to <400>30 under low stringency conditions at 42° C. or a variant, mutant or homologue of said actinomycete.
(ap) The actinomycete of (ao) wherein said actinomycete corresponds to PM342.

38. Metabolites derived from the novel actinomycetes according to claims 36 or 37 and derivatives, homologues, analogues, chemical equivalents, mutants and mimetics of said metabolites.

39. An antibody directed to the actinomycete of claims 36 or 37 or the metabolite of claim 37 or derivative, homologue, analogue, chemical eqivalent or mimetic of said antibody.

40. A method of improving plant productivity said method comprising introducing into said plant or propagation material thereof:
(i) An effective number of actinomycetes according to claims 36 or 37 or variants, mutants or homologues thereof and/or
(ii) An effective amount of one or more metabolites derived from the actinomycetes of (i) or derivative, homologue, analogue, chemical equivalent and mimetic thereof.
for a time and under conditions sufficient to induce, in the subject plant, said characteristic.

41. A method of facilitating the biodegradation of biodegradable material, said method comprising contacting said waste material with:
(i) An effective number of actinomycetes according to claims 36 or 37 or variants, mutants or homologues thereof and/or
(ii) An effective amount of one or more metabolites derived from the actinomycetes of (i) or derivative, homologue, analogue, chemical equivalent and mimetic thereof
for a time and under conditions sufficient to induce or otherwise facilitate the degradation of said material.

42. A method for therapeutically and/or prophylactically treating a condition in a subject, the aberrant, unwanted or otherwise inappropriate symptoms, causes or outcomes of which condition are treatable with one or more metabolites derived from the actinomycetes of claims 36 or 37, said method comprising to said subject an effective amount of one or more of said metabolites or derivatives, homologues, analogues, chemical equivalents or mimetics thereof for a time and under conditions sufficient to ameliorate said symptom, cause or outcome.

43. Use of the novel actinomycete of claims 35 or 36 or metabolites of claim 37 in the manufacture of a medicament for the therapeutic and/or prophylactic treatment of a mammalian or non-mammalian subject.

44. Use according to claim 43 wherein said non-mammalian subject is a plant.

45. Use of the novel actinomycete of claims 36 or 37 or the metabolite of claim 38 in the manufacture of a composition for agricultural application.


SEQUENCE LISTING (33 sequences)

SEQ ID NO: 1 - DNA, 1158 bp, actinomycete
  misc_feature (1)..(1155): “n” is unknown nucleotide
cttaacacatgcaantcaagcggaaaggcccttcggggtactcaancggcnaacgggtgattaacacntgantaacctgcccctgactctgggataancctgggaaactgggtctaataccggatacaaccatttctcncatgggatggtggtggaaantttttncggttggggatgggctcgcggcctatcaccttgttggtggggtgatggcctaccaaggcgacgaacggtagcccgcctgagagggcgaccggccacactgggactgagacaccgcccgaactcctacgggaggcagcactggggaatattgcccatgggcggaagcctgacgcagngacgccgcgtgggggatgacggccttngggttgtaaacctntttcagcagggacgaagttgacgtgtacctgtagaagaagcgccggctaaatangtgccagcagccgcggtaatangtagggcgcgagcgttntccggaattattgggcgtaaagagtttgtaggtggcttgttgcgtttgccgtgaaagcccgtggcttaantacgggtttgcggtggatacgggcaggctagaggctggtaggggcaagcggaattcctggtgtagcggtgaaatgcgcagatatcaggaggaacaccggtggcgaaggcggcttgctgggccagttctgacggtgaggagcgaaagcgtggggagcgaacaggattagataccctggtagtccacgctgtaaacgttgggcgctaggtgtgggggtcttccacgatctctgtgccgtagctaacgcattaagcgccccgcctggggagtacggccgcaaggctaaaactcaaaggaattgacgggggcccgcacaagcggcggagcatgttgcttaattcgacgcaacgcgaagaaccttaccaaggtttgacatacaccggaaacactcananatgggtgcctcctttggactggtgtacaggtggtgcatggctgtcnncaccctcgtgtcgtnagatgtngggttaagtcccgcaacgancgcaacccttggttccatgttgccagcacnccctttgnggtggtggggacncatggganaatgccggggtcnactcnggaggaaggtggggatgacgtcaagtnatcntgccccttatgttcttgnngtg

SEQ ID NO: 2 - DNA, 1437 bp, actinomycete
  misc_feature (1)..(1437): “n” is unknown nucleotide
gctggcggcgtgcttaacacatgcaagtcgaacgatgaaccacttcggtggggattagtggcgaacgggtgagtaacacgtgggcaatctgcccttcactctgggacaagccctggaaacggggtctaataccggataacactnctgctctcatgggcaggggttaaaagctccggcggtgaaggatgagcccgcggcctatcagcttgttggtgaggtaatggctcaccaaggcgacgacgggtagccggcctgagagggcgaccggccacactgggactgagacacggcccagactcctacgggaggcagcagtggggaatattgcaacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccggaattattgggcgtaaagagcttgtaggcggcttgtcacgtcgggtgtgaaagcccggggcttaaccccgggtctgcattcgatacgggctagctagagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaagcatcagagatggtgccccccttgtggttcggtgtacaggtggtgcatggctgtcgtcagctcgtgtcgtgagatgttgggttaagtcccgcaacgagcgcaacccttgttctgtgttgccagcatgcccttcggggtgatggggactcacaggagaccgccggggtcaactcggaggaaggtggggacgacgtcaagtcatcatgccccttatgtcttgggctgcacacgtgctacaatggccggtacaaagagctgcgataccgtgaggtggagcgaatctcaaaaagccggtctcagttcggattggggtctgcaactcgaccccatgaagtcggagttgctaataatcgcanatcagcattgctgcggtgaatacgttcccgggccttgtacacaccgcccgtcacgtcacgaaagtcggtaacacccgaagccggtggccaaccccttgtgggagggagctgtcgaaggtgggactggcgattg

SEQ ID NO: 3 - DNA, 317 bp, actinomycete
  misc_feature (1)..(311): “n” is unknown nucleotide
gtaatggcccanaaaaccgccttcgccaccggtgttcctcctgatatctgcgcatttcaccgctacaccaggaattccnatctcccctaccacactctagctagcccgtatcnaatgcaaactcggggttaagccccnagctttcacatccgacgtgacaagccgcctacaanctctttacgcccaataattccgganaacgctcgcaccctacntnttaccgcggctgctggcncgtntttagccggtgcttcttctgcaggtaccgtcactttcgcttcttccctgctnaaaaaggtttacaaccctanggccgt

SEQ ID NO: 4 - DNA, 1048 bp, actinomycete
  misc_feature (1)..(1043): “n” is unknown nucleotide
tgagggatgacggcnttcggggttgtaaacntttntcaccagggaagaagcgaaagtgncggtacctgcagaagaagcgccgnctaactacgggccagcatccgcggtaatacgtagggcgcaatcgttgtccggaattantgggcgtaaagagntcgtaggcggcttatcacgtcgggtgtgaaagcccggggcttaagccccgggtctgcattcgatacgggctagctagantntgntaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaagcatcagagatggtgccccccttgtggtcggtgtacaggtggtgcatggctgtcgtcagctcgtgtcgtgagatgttgggttaagtcccgcaacgagcgcaacccttggttctgtgttgccagcatgcccttcggggtgatggggactcacaggagaacgccggggtcaactcggaggaaggtggggacgacgtcaagtcatcatgccccttatgtcttgggctgcacacgtgctacaatggcaggtaaatgagctgcgataccgtgaggtggagcgaatctcaaaaaagcctgtctcanttcggattggggtctgnaantcgaccccatgaaagtcggagttgctaattatcccagatcaacattgctggcggtgaatacgttcccggggccttggtaaacaccgcccgtcaangtnaagaaagtcgggtaacacccgaaanccggtgggccaancccct

SEQ ID NO: 5 - DNA, 508 bp, actinomycete
  misc_feature (1)..(472): “n” is unknown nucleotide
ccgccttcgccaccgggtgttcctcctgatatctgcgcatttcaccgctacaccaggaaattccnatctcccctaccacactctanctancccgtatcgaatgcaaacccggggttaanccccgggctttcacacccgacntgacaagccgcctacaaactctttacgcccaataattccggacaacgcttgcgccctacntattaccgcggctgctggcacntatttagccggcgcttcttctgcaggtaccgtcactttcgcttcttccctgctgaaaaaggtttacaacccgaaggccgtcatccctcacgcggcgtcgctgcatcaggctttcgcccattgtgcaatattccccactgctgcctcccntaggaatctgggccgtgtctcaatccagtgtggccggtcccctctcnggccggctaccgtcntcccttggtnaccattanctcaccaacaactgataggncgcgggctcatcttcacgcgggaactttcaaccacc

SEQ ID NO: 6 - DNA, 1420 bp, actinomycete
ggcggcgtgcttaacacatgcaagtcgaacgatgaagcccttcggggtggattagtggcgaacgggtgagtaacacgtgggcaatctgcccttcactctgggacaagccctggaaacggggtctaataccggatacgattcgggaggcatctcctggtactggaaagctccggcggtgaaggatgagcccgcggcctatcagcttgttgtgggtaatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgagagtgacggtacctgcagaagaagcgccggctaactacgtgccagcagccgcggtaatacgtagggcgcaagcgttgtccggaattattgggcgtaaagagctcgtaggcggcttgtcacgtcgggtgtgaaagcccggggcttaaccccgggtctgcatccgatacgggcaggctagagtgtggtaggggagatcggaattcctggtgtagcggtgaaatgcgcagatatcaggaggaacaccggtggcgaaggcggatctctgggccattactgacgctgaggagcgaaagcgtggggagccaacaggattagataccctggtagtccacgccgtaaacgttggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatataccggaaagcgccagagatggtgccccccttgtggtcggtatacaggtggtgcatggctgtcgtcagctcgtgtcgtgagatgttgggttaagtcccgcaacgagcgcaacccttgtcctgtgttgccagcatgcccttcggggtgatggggactcacaggagaccgccggggtcaactcggaggaaggtggggacgacgtcaagtcatcatgccccttatgtcttgggctgcacacgtgctacaatggccggtacaaagagctgcgatgccgtgaggcggagcgaatctcaaaaagccggtctcagttcggattggggtctgcaactcgaccccatgaagtcggagttgctagtaatcgcagatcagcattgctgcggtgaatacgttcccgggccttgtacacaccgcccgtcacgtcacgaaagtcggtaacacccgaagccggtggcccaacccctcggggagggagctgtcgaaggtgggac

SEQ ID NO: 7 - DNA, 1239 bp, actinomycete
  misc_feature (1)..(1217): “n” is unknown nucleotide
gcttnttggtgggncnatggcctaccaaggngaggacggntanccngcctgngagggagaccgnccacactgggaatgnganacggcccagaatcctacgggaggcagcannggggaanattgcacaangggcgaaagcctgatgcagngangccgcgtgagggaagacggcctttgggttgtaaacctntttnagcagggaagaagcgaaagtgacggtacctgcagaagaagcgccggctaantangtgccagcagccgcggtaatangtagggcgcaagcgttgtccggaattattgggcgtaaagagcttgtaggcggcttgtcangtnggatgtgaaagcccggggcttaaccccgggtttgcatttgatacgggctagctagagtgtggtaggggagatnggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacgttgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatataccggaaagcatcagagatggtgccccccttgtggtcggtatacaggtggtgcatggctgtcgtcagctcgtgtcgtgagatgttgggttaagtcccgcaacgagcgcaacccttgttctgtgttgccagcatgcccttcggggtgatggggactcacaggagactggcggggtcaactcggaggaaggtggggacgacgtcaagtcatcatgccccttatgtcttggggctgcacacgtgctacaatggccggtacaatgagctgcgatgccgcgaggcggagcgaatctcaaaaagccggtctcagttcggattgggggtctgnaactcgaccccatgaantcggagttgctaataatcccaaattcancattggtgcggtgaatacttcccgggcctggtacacnaccgcccgtcaactcacgaaagtcggtnaaacccgaaaccggtgggccaaccccttgtgggaaggaactggccnaagtgggactggcgattgggac

SEQ ID NO: 8 - DNA, 431 bp, actinomycete
  misc_feature (1)..(424): “n” is unknown nucleotide
ccgccttcgccaccggtgttcctcctgatatctgcgcatttcaccgctacaccaggaattccnatctcccctaccacactctagctagcccgtatcaaatgcaaacccggggttaagccccgggctttcacatccnacgtgacaagccgcctacaanctctttacgcccaataattccggacaacgcttgcgccctacntattaccgcggctgctggcacntatttagccggcgcttcttctgcaggtaccgtcactttcgctncttccctgctgaaanaggtttacaacccaaaggccntcatccctcnccggcntcnttgcntcnggcttncncccattgttcaannttccccactgctncctcccctcggaatctgggccgntgtctcattcccnttntggccggtccccctcncaggccngctaccc

SEQ ID NO: 9 - DNA, 653 bp, actinomycete
  misc_feature (1)..(640): “n” is unknown nucleotide
ctcagcgtcngtaatggcccaaaaaccgccttcgccaccggtgttcctcctgatatctgcgcatttcaccgctacaccaggaattccnatctcccctaccacactctagctagcccgtatcnaatgcaaacccggggttaanccccgggctttcacatccnacntgacaagccgcctacaanctctttacgcccaataattccggacaacgcttgcnccctacttattaccgcggctgctggcacttatttagccggcgcttcttctgcaggtaccgtcactttcgcttcttccctgctnaaaaaggtttacaacccnaaggccgtcatccctcacgcggcntcgctgcatcaggctttcncccattgtgcaatattccccactgctgcctcccgtaggattctgggccgtntctcattcccantgtggccggtcgccctctcaggccggctacccgtcntcnccttggtaggccattaccccaccaacaagctnataggccgcgggctcatccttcaccgccggaagctttcaaccccntccatgcggganaaattgttntccggtattaaaccccgtttccagggnttgtcccaaaattgaaggggggattgnccactttttactcacccgttcnccnctaatccaccacc

SEQ ID NO: 10 - DNA, 1444 bp, actinomycete
acgaacgctggcggcgtgcttaacacatgcaagtcgaacgatgaagccgcttcggtggtggattagtggcgaacgggtgagtaacacgtgggcaatctgcccttcactctgggacaagccctggaaacggggtctaataccggataacactctgtcccgcatgggacggggttgaaagctccggcggtgaaggatgagcccgcggcctatcagcttgttggtggggtaatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccggaattattgggcgtaaagagctcgtaggcggcttgtcacgtcggatgtgaaagcccggggcttaaccccgggtctgcattcgatacgggctagctagagtgtggtaggggagatcggaattcctggtgtagcggtgaaatgcgcagatatcaggaggaacaccggtggcgaaggcggatctctgggccattactgacgtctgaggagcgaaagcgtggggagcgaacaggattagataccctggtagtccacgccgtaaacgttgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatataccggaaagcatcagagatggtgccccccttgtggtcggtatacaggtggtgcatggctgtcgtcagctcgtgtcgtgagatgttgggttaagtcccgcaacgagcgcaacccttgttctgtgttgccagcatgcccttcggggtgatggggactcacaggagactgccggggtcaactcggaggaaggtggggacgacgtcaagtcatcatgccccttatgtcttgggctgcacacgtgctacaatggccggtacaatgagctgcgatgccgcgaggcggagcgaatctcaaaaagccggtctcagttcggattggggtctgcaactcgaccccatgaagtcggagttgctagtaatcgcagatcagcattgctgcggtgaatacgttcccgggccttgtacacaccgcccgtcacgtcacgaaagtcggtaacacccgaagccggtggcccaacccttgtgggagggagctgtcgaaggtgggactggcgattg

SEQ ID NO: 11 - DNA, 503 bp, actinomycete
  misc_feature (1)..(499): “n” is unknown nucleotide
ccgccttcgccaccggtgttcctcctgatatctgcgcatttcaccgctacaccaggaattccnatctcccctaccgaactctancctgcccgtatcnactgcaaacccggggttaagccccgggctttcacaaccgacntgacaagccgcctacaanctctttacncccaataattccggacaacgcttgcgccctacntattaccgcggctgctggcacntatttagccggcgcttcttctgcaggtaccgtcactttcgcttcttccctgctgaaaaaggtttacaacccgaaggccgtcntccctcacgcggcgtcgctgcatcaggctttcgcccattgtgcaatattccccactgctgcctcccgtaggattctgggccgtgtctcantcccantntggccggtcccctctcaggccggntacccgtcgtcccttggtgaaccnctacctcnccaacaanctgatagggcgcgggctcancntgcacgccggancttt

SEQ ID NO: 12 - DNA, 1173 bp, actinomycete
  misc_feature (1)..(1144): “n” is unknown nucleotide
ttaanacatgcaantcgaacgatgaacccngtttcggtggtggattagtggcgaacggtgagtaanangtgggcaatttgcccttcattttggacaagccctggaaacgggtttaataccggataacattttntcccgcatggganggggttgaaagntccggcggtgaaggatgagcccgcggcctatnagcttgttggtggggtaatggcctacccaagggagacgggtagccggcctgagagggcgaccggccacactgggaatgaganacggcccagaatcctacgggaggcagcagtggggaatattgcacaatgggcgaaagcctgatgcagcgangccgcgtgagggatgacggccttngggttgtaaacctttttnagcagggaagaagcgaaagtgacggtacctgcagaagaagcgccggctaaataagtgccagcagccgcggtaataagtagggcgcaagcgttgtccggaattattgggcgtaaagagcttgtaggcggcttgtcangtnggatgtgaaagcccggggnttaaccccgggtttgcatttgatacgggctagntagagtgtggtaggggagatnggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacgttgggaactaggtgttggcgacattccacgtcgtcggtgccgcagctaacgcattaagttccccgnctggggagtacggccgcaaggctaanactcaaaggaattgacgggggcccgnacaagcagcggancatgtggcttaattcgacgcancgcgaagaaccttaccaaggcttgacatataccggaaagcatcagagatggtgccccccttgtggtcgntatacangtggtgcatgnctgtcgtcacctcgtgtcgtgagatgttgggttaagtcccgcaacgagcgcnacccttgntctgtgttgncancatgcccttcggggntgatggggactcacagganactgnccggggtcaactccggangaaggtgggtgacgaagtcaaggtcatcatgnccccttatgtcttggtgctgcacacgtgc

SEQ ID NO: 13 - DNA, 1404 bp, actinomycete
  misc_feature (1)..(493): “n” is unknown nucleotide
ttcggnggtggantagnggcgnacgggngaccaacangngggcaatccccccttcantttnggacaacccctggaaacgggttntaataccggataacantttntccccgcatggganggggttgaaagctccggcggtgaaggatgagcccgcggcctatcagcttgttggtggggtaatggcctaccaaggcgacgacgggtagccggcctgagagggcgaccggccacactgggantgaganacggcccagaatcctacgggaggcagcagtggggaatattgcacaatgggcgaaagcctgatgcagcgacgccgcgtgagggatgacggccttcgggttgtaaacctttttcagcagggaagaagcgaaagtgacggtacctgcagaagaagcgccggctaaatangtgccagcagccgcggtaatangtagggcgcaagcgttgtccggaattattgggcgtaaagagnttgtaggcggcttgtcangtcggatgtgaaagcccggggcttaaccccgggtttgcattcgatacgggctagctagagtgtggtaggggagatcggaattcctggtgtagcggtgaaatgcgcagatatcaggaggaacaccggtggcgaaggcggatctctgggccattactgacgctgaggagcgaaagcgtggggagcgaacaggaattagataccctggtagtccacgccgtaaacgttgggaactaggtgttggcgacattccacgtcgtcggtgccgcagctaacgcattaagttccccgcctggggagtacggcccgcaaggctaaaactcaaaggaattgacgggggcccgcacaagcagcggagcatgtggcttaattcgacgcaacgcgaagaaccttaccaaggcttgacatataccggaaagcatcagagatggtgccccccttgtggtcggtatacaggtggtgcatggctgtcgtcagctcgtgtcgtgagatgttgggttaagtcccgcaacgagcgcaacccttggttctgtgttggccagcatgcccttcggggtgatggggactcacaggagactggccggggtcaactcggaggaaggtggggacgacgtcaagtcatcatgccccttatgtcttggggctgcacacgtgctacaatggccggtacaatgagctgcgatgccgcgaaggcggagcgaatctcaaaaaagccggtctcagttcggattggggtctgcaactcgaccccatgaagtcggagttgctagtaatcgcagatcagcattgctgcggtgaatacgttcccgggccttgtacacaccgcccgtcacgtcacgaaagtcggtaacacccgaagccggtggtccaaccccttgtgggagggagctgtcgaaggtgggactggcgattgg

SEQ ID NO: 14 - DNA, 1411 bp, actinomycete
  misc_feature (1)..(1411): “n” is unknown nucleotide
aacacatgcaagtcgaacgatgaagccgcttcggtggtggattagtggcgaacgggtgagtaacacgtggccaantgtgnccgtcactatgggacgaagaccttggaaacggggtctaataccggataacactctgtcccgcatgggacggggttgaaagctccggcggtgaaggatgagcccgcggcctatcagcttgttggtggggtaatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccggaattattgggcgtaaagagctcgtaggcggcttgtcacgtcggatgtgaaagcccggggcttaaccccgggtctgcattcgatacgggctagctagagtgtggtaggggagatcggaattcctggtgtagcggtgaaatgcgcagatattcaggaggaacaccggtggcgaaggcggatctctgggccattactgacgctgaggagcgaaagcgtggggagcgaacaggattatataccctggtagtccacgccgtaaacgttgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatataccggaaagcatcagagatggtgccccccttgtggtcggtatacaggtggtgcatggctgtcgtcanctcgtgtcgtgagatgttgggttaagtcccgcaacgagcgcaacccttgttctgtgttgccagcatgcccttcggggtgatggggactcacaggagactgccggggtcaactcggaggaaggtggggacgacgtcaagtcatcatgccccttatgtcttgggctgcacacgtgctacaatggccgctacaatgacctgcgatgccgcgaggcggaccgaatctcaaacaagcccgtctcattcggattgcggtctgcaactccgaccccatgaagtccgacttgctagtactcgcacgtcaacattgctgcgctgaatacgtccccgggccttgtacacaccgcccgtcacgtcacgaaagtcggtaacacccgaagccggtggnccaaccccttgtgggagggagctgtcgaa

SEQ ID NO: 15 - DNA, 562 bp, actinomycete
  misc_feature (1)..(547): “n” is unknown nucleotide
ccgccttcgccaccggtgttcctcctgatatctgcgcatttcaccgctacaccaggaattccnatctcccctaccacactctagctancccgtatcnaatgcaaacccggggttaacccccgggctttcacacccnacntnacaanccgcctacaaactctttacgcccaataattccggacaacgcttgcgccctacttattaccgcggctgctggcacttatttagccggcgcttcttctgcaggtaccgtcactttcgcttcttccctgctgaaaaaggtttacaacccgaaggcngtcatccctcacgcggcntcgctgcatcaggctttcgcccattgtgcaatattccccactgctgcctcccgtagnantctgggccgtntctcantcccagtgtggncggtcgccctctcaggccggctacccgtcgtcncctnggtnaaccattanntcaccaacaagctgataggccgcgggctcatccttcaccgccggagcttttaacccctgcccatgaaaacagangtnttatccggtattanaacccgtttccaggg

SEQ ID NO: 16 - DNA, 1390 bp, actinomycete
  misc_feature (1)..(1362): “n” is unknown nucleotide
atgcaagtcgagcggaaaggcccttcggggtactcgagcggcgaacgggtgagtaacacgtgagttaatctgccccaggctctggatacccaccggaaaacggtgattaataccgaatacgacaaccgatttgcatgatctggtggtgnaaagtttttcggcctgggatgtgcttcgcggcctatcagcttgttggtgaggtaatggctcacccaaggcttcgacggtagccggcctgagagggtgaccgnccacactgggactgagacacggcccagactcctacgggaggcagcagtggggaatattggacaatgggcggaagcctgatccagcaacgccgcgtgagggatgacggccttcgggttgtaaacctctttcagcacagacgaagcgcaagtgacggtatgtgcagaagaaggaccggccaactacgtgccagcagccgcggtaatacgtagggtccgagcgttgtccggaattattgggcgtaaagggctcgtaggcggtctgtcgcgtcgggagtgaaaaccaggtgcttaacacctggcctgctttcgatacgggcagnctagaggtacncaggggagaatggaattcctggtgtagcggtgaaatgcgcagatatcaggaggaaacaccggtggcgaagncggttctctgggagtatcctgacgctgaggagcgaaagtgtggggagcgaacaggattagataccctggtagtccacaccgtaaacgttgggcgctaggtgtgggacacattccacgtgttccgtgccgcagctaacgcattaancgccccgcctggggagtacggccgcaangctaaaactcanaggaattgacgggggcccgcacaagcggcggagcatgcggattaattcgatgcaacgcgaagaaccttacctgggtttgacatacaccggaaagccgtacagatacggccccttttagtcggtgtacaggtggtgcatggctgtcgtcagctcgctgtcgtgagatgttcgggttaagtcccgcaacgagcgcaaccctcgtcctatgttgccagcaattcggttggggactcataggagactgccggggtcaactcggaggaaggtggggatgacgtcaagtcatcatgccccttatgtccagggcttcacgcatgctacaatggccggtacaaagggctgcgatcccgtgagggtgagcgaatcccaaaaagccggtctcagttcggattggggtctgcaactcgaccccatgaagtcggagtcgctagtaatcgcagatcagcaacgctgcggtgaatacgttcccgggccttgtacacaccgcccgtcacgtcacgaaagtcggcaacacccgaagccantggcccaactcgtaagagagggagctgt

SEQ ID NO: 17 - DNA, 1411 bp, actinomycete
  misc_feature (1)..(638): “n” is unknown nucleotide
gtgcttaacacatgcaagtcgaacgatgaagccgcttcggtggtggattagtggcgaacgggtgagtaacacgtgggcaatctgcccttcactctgggacaagccctggaaacggggtctaataccggataacactctgtcccgcatgggacggggttgaaagctccggcggtgaaggatgagcccgcggcctatcagcttgttggtgggtaatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccggaattattgggcgtaaagagctcgtaggcggcttgtcacgtcggatgtgaaagcccggggcttaaccccgggtctgcattcgatacgggctagctagagtgtggtaggggagatcggaattcctggtgtagcggtgaaatgcgcagatatncaggaggaacaccggtggcgaaggcggatctctggccattactgacgctgaggagcgaaagcgtggggagcgaacaggattagataccctggtagtccacgccgtaaacgttgggaactaggtgttggcgacattccacgtcgtcggtgccgcagctgaacgcattaagttccccgcctggggagtacggccgcaaggctaaaactcaaaggaattgacgggggcccgcacaagcagcggagcatgtggcttaattcgacgcaacgcgaagaaccttaccaaggcttgacatataccggaaagcatcagagatggtgccccccttgtggtcggtatacaggtggtgcatggctgtcgtcagctcgtgtcgtgagatgttgggttaagtcccgcaacgagcgcaacccttgttctgtgttgccagcatgcccttcggggtgatggggactcacaggagactgccggggtcaactcggaggaaggtggggacgacgtcaagtcatcatgccccttatgtcttgggctgcacacgtgctacaatggccggtacaatgagctgcgatgccgcgaggcggagcgaatctcaaaaagccggtctcagttcggattggggtctgcaactcgaccccatgaagtcggagttgctagtaatcgcagatcagcattgctgcggtgaatacgttcccgggccttgtacacaccgccgtcacgtcacgaaagtcggtaacacccgaagccggtggcccaaccgccttgtgggagggaactttcca

SEQ ID NO: 18 - DNA, 1370 bp, actinomycete
  misc_feature (1)..(1367): “n” is unknown nucleotide
atgcaagtngaacgatgaanccntttggggtggattagtggcgaacgggtgagtaanangtgggcaatttgcccttcaatttgggacaagccctggaaacggggtntaataccggataacantntgtcccgcatgggacggggttaaaagctccggcggtgaaggatgagcccgcggcctatnagcttgttggtggggtgatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctttttcagcagggaagaagcgaaagtgacggtacctgcagaagaagcgccggctaaatangtgccagcagccgcggtaatangtagggcgcaagcgttgtccggaattattgggcgtaaagagtttgtaggcggcttgtcacgtnggatgtgaaagcccggggcttaaccccgggtttgcattcgatacgggctagctagagtgtggtaggggagatcggaattcctggtgtagcggtgaaatgcgcagatatcaggaggaacaccggtggcgaaggcggatctctgggccattactgacgntgaggagcgaaagcgtggggagcnaacagnattagataccctggtagtccaagccgtaaacgttgggaactangtgttggcgacattccacgtcgtcnntgccgcanctaacgcattaagttccccgcctggggagtacggccgcaaggctaanactcaaaggaattgangnnggcccgcacaagcagcggagcatgtggcttanttcnacgcancgcgaagaaccttaccaaggtttgccatataccggaaagcatcagagatggtgccccccttgtggtcggtatacaggtggtgcntggctgtcgtcagctcgtgtcgtgacatgttggttaagtcccgtcaacgaggcgcaacccttgttntgtgtngccagcatgcccttcggggtgatggggactcacaggagactgccggggtcaactcggaggaaggtggggacgacgtcaagtcatcatgccccttatgtcttgggctgcacacgtgctacaatggccggtacaatgagctgcgatgccgcgaggcggagcgaatctcaaaaagccggtntcagttcggattggggtctgcaactcgaccccatgaagtcggagttgctagtaatcgcagatcagcattgctgcggtgaatacgttcccgggccttgtacacaccgcccgtcacgtcacgaaagtcggtaacacccgaagccgntgg

SEQ ID NO: 19 - DNA, 1162 bp, actinomycete
  misc_feature (1)..(1156): “n” is unknown nucleotide
gaacgatgaagccgtttcggtggtggattagtggcgaacggtgagtaaaagtggcaatttncccttcattttggacaagccctggaaacgggtttaanaccggataacattntgtcccgcatgggacggggttgaaagntcccggcggtgaaggatgagcccgcggcntatcagcttgttggtggggtaatggcctaccaaggcgacgacgggtagccggcctgagagggcgaccggccacactgggantgagacacggcccagactcctacgggaggcagcagtggggaatattgcacaatgggcgaaagcctgatgcagcgacgccgcgtgagggatgacggccttcgggttgtaaacctntttcagcagggaagaagcgaaagtgacggtacctgcagaagaagcgccggctaaatangtgccagcagccgcggtaatangtagggcgcaagcgttgtccggaattattgggcgtaaagagcttgtaggcggcttgtcangtcggatgtgaaagcccggggcttaaccccgggtttgcattcgatacgggctagttagagtgtggtaggggagatnggaattcctggtgtagcggtgaaatgcgcagatatcaggaggaacaccggtggcgaaggcggatctctgggccattactgacgctgaggagcgaaagcgtggggagcnaacaggattagataccctggtagtccacgccgtaaacgttgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaacaaccttaccaaggcttgacatataccggaaagcatcanagatggtgccccccttgtggtcggtatacangtggtgcatggctgtcgtcagctcgtgtcgtgagatgttgggttangtcccgcaacgagcgcnacccttgttctgtgtcgncnagcatgcccttcgnggtgatggggactcacangagactgncggggtccactcggaggaaggtggcgacnacgtcanntcatcatgcccccttatgtcttgggnctggccacgtgcnacnatggcc

SEQ ID NO: 20 - DNA, 1411 bp, actinomycete
  misc_feature (1)..(1404): “n” is unknown nucleotide
gctggcggcgtgcttaacacatgcaagtcgaacgatgaagccgcttcggtggtggattagtggcgaacgggtgagtaacacgtgggcaatctgcccttcactctgggacaagccctggaaacggggtctaataccggataacactctgtcccgcatgggacggggttgaaagctccggcggtgaaggatgagcccgcggcctatcagcttgttggtggggtaatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccggaattattgggcgtaaagagctcgtaggcggcttgtcacgtcggatgtgaaagcccggggcttaaccccgggtctgcattcgatacgggctagctagagtgtggtaggggagatcggaattcctggtgtagcggtgaaatgcgcagatatcaggaggaacaccggtggggaaggcggatctctgggccattactgacgctgaggagcgaaagcgtggggagcgaacaggattagataccctggtagtccaagccgtaaacgttgggaactangtgttggcgacattccacgtcgtcggtgccgcagctaacgcattaagttccccgtcctggggagtacggccgcnaggctaaaactcaaaggaattgacgggggcccgcacaagcagcggagcatgtggcttanttcgacgcnacgcgaagaaccttnccaaggctgacatataccggaaagcatcacagatggtgccccccttgtggtcggtatacagggtggtgcatggctgttcgtcagctcgtgtcgtgagatgttgggttaagtcccgcaaagagcgcaaccgtgttctgtgttgccagcatgcccttcggggtgatggggactcacacgagactgtcngggtcaactcggaggaaggtggggacgacgtcaagttcatcatgccccttatgtcttgggctgcacacgngctacaatggccggtacaatgagnngggatgccgcgaggcggagcgaatctcaaaaagccggtctcagttcggattggggtctgcaactgaccccatgaagtcggagttgctagtaatcgcagatcagcattgctgcggtgaatacgtncccgggcctngtacacaccacccgtcacgtcacgaaagtcggtaacaccctaagccggtgncccaaccccttntgggagg

SEQ ID NO: 21 - DNA, 549 bp, actinomycete
  misc_feature (1)..(431): “n” is unknown nucleotide
ccaganatccgccttcgccaccggtgttcctcctgatatctgcgcatttcaccgctacaccaggaattccgatctcccctaccacactctagctagcccgtatcgaatgcagacccggggttaagccccgggctttcacatccgacgtgacaagccgcctacgagctctttacgcccaataattccggacaacgcttgcgccctacgtattaccgcggctgctggcacgtagttagccggcgcttcttctgcaggtaccgtcactttcgcttcttccctgctgaaagaggtttacaacccgaaggncgtcatccctcacgcggcgtcgctgcatcaggctttcgcccattgtgcaatattccccactgctgcctcccgtaggagtctgggncgtgttcaatnccagtggtgggccggtcgccctctcaggncggctaccgtcgtcgccttggtaggcattaccacaacaagctgataggcgggggtcatccttcaacgccggagcttcaaacccgtccatgcgggacaagtgtatccggtattaaaccc

SEQ ID NO: 22 - DNA, 672 bp, actinomycete
  misc_feature (1)..(643): “n” is unknown nucleotide
tcagtnatggcccagaangatccgncttcgccaccggtgttcctcctgatatctgcgcatttcaccgctacaccaggaattccgatctcccctaccacactctaactagcccgtatcgaatgcagacccggggttaagccccgggctttcacatccgacgtgacaagccgcctacgagctcttnacgcccaataattccggacaacgcttgcgccctacgtattaccgcggctgctggcacgtagttagccggcgcttcttctgcaggtaccgtnactttcgcttcttccctgctgaaagaggtttacaacccgaaggccgtcntccctcacgcggcgtcgctgcatcaggctttcgcccatngtgcantattccccactgntgnctcccgtangagtctgggccgtgtctcagtcccagtgtggccggtcgncctctcaggccggctaccgtcgtcgccttggtaggncattacccaccaacaagctgatangtcgngggctcatccttcaccgncggagntttaaccccgtncatgcgggacagagtgttatccggtattanacccgtatncagggcttgtcccatagtgaagggnagatngccacgtgttatcaccgttcgncactaatnatcancgaancggcttcatcgttcgacttgcatgtgtta

SEQ ID NO: 23 - DNA, 678 bp, actinomycete
  misc_feature (1)..(648): “n” is unknown nucleotide
ctcagcgtcagtcatggccaagagatccgccttcgccaccggtgttcctcctgtatatctgcgcatttcaccgctacaccaggaattccgatctcccctaccacactctagctagcccgtatcgaatgcagacccggggttaagccccgggctttcacatccgacgtgacaagccgcctacgagctctttacgcccaataattccggacaacgcttgcgccctacgtattaccgcggctgctggcacgtagttagccggcgcttcttctgcaggtaccgtcactttcgcttcttccctgctgaaagaggtttacaacccgaaggccgtcatccctcacgcggcgtcgctgcatcaggctttcgcccattgtgcaatattccccactgctgcctcccgtaggagtctgggccgtgtctcagtcccagtgtggccggtcgccctctcaggccggctacccgtcgtcgccttggtaggccattacccaccaacaagctgataggccgcgggctcatccttcancgncggagctttaacccgtccatgcgggacagagtgttatccggtattaaacccgtttcagggcttgtcccanagtgaagggcagattgccacgtgttatcanccgttcgncactaatcacancgaancgggttcatcgttcgacttgcatgtgttaa

SEQ ID NO: 24 - DNA, 688 bp, actinomycete
  misc_feature (1)..(666): “n” is unknown nucleotide
ggcccaganatccgncttcgccaccggtgttcctcctgaatatctgcgcatttcaccgctacaccaggaattccgatctcccctaccacactctaactagcccgtatcgaatgcagacccggggttaagccccgggctttcacatccgacgtgacaagccgcctacgagctctttacgcccaataattccggacaacgcttgcgccctacgtattaccgcggctgctggcacgtaattagccggcgcttcttctgcaggtaccgtcactttcgcttcttccctgctgaaagaggtttacaacccgaaggccgtcatccctcacgcggcgtcgctgcatcaggctttcgcccattgtgcaatattccccactgctgnctcccgtangagtctgggccgtgtctcagtcccagtgtggccggtcgncctctcaggccggctaccgtcgtcgccttggtaggccattaccccaccaacaagctgatangccgngggctcatccttcancgtcggagctttcaancccgtccatgcgggacagagtgttatccggtattanaccccgtntcagggcttgtccanagtgaagggcagatngccacgtgttatcaccgttcgccactaatnacancgaaacggcttatcgtncgactgcatgtgttaacacncgcagcgttcgtcctgagccag

SEQ ID NO: 25 - DNA, 702 bp, actinomycete
  misc_feature (1)..(658): “n” is unknown nucleotide
ccctcagggtcagtaatgggcccagagatccgccttcgccaccggtgttcctcctgaatatctgcgcatttcaccgctacaccaggaattccgatctcccctaccacactctagctagcccgtatcgaatgcagacccggggttaagccccgggctttcacatccgacgtgacaagccgcctacgagctctttacgcccaataattccggacaacgcttgcgccctacgtattaccgcggctgctggcacgtagttagccggcgcttcttctgcaggtaccgtcactttcgcttcttccctgctgaaagaggtttacaacccgaaggccgtcatccctcacgcggcgtcgctgcatcaggctttcgcccattgtgcaatattccccactgctgcctcccgtaggagtctgggccgtgtctcagtcccagtgtggccggtcgccctctcaggccggctanccgtcgtcgccttgggtaggcattancccancaacaagctgataggncgcgggctcatncttcaacgccggagctttcaancccgtccatgcgggacagagtgttatncggtattaaacccgtttcagggcttgttccagagtgaagggcagattgccacgtgttatcaaccgttcggcactaatcacaacgaagcggnttatcgttcgacttgcatgtgttaacaagccgccagcgttcgtc

SEQ ID NO: 26 - DNA, 711 bp, actinomycete
  misc_feature (1)..(687): “n” is unknown nucleotide
tcagtaatggcccagagatccgccttcgccaccggtgttcctcctggatatctgcgcatttcaccgctacaccaggaattccgatctcccctaccacactctagctagcccgtatcgaatgcagacccggggttaagccccgggctttcacatccgacgtgacaagccgcctacgagctctttacgcccaataattccggacaacgcttgcgccctacgtattaccgcggctgctggcacgtagttagccggcgcttcttctgcaggtaccgtcactttcgcttcttccctgctgaaagaggtttacaacccgaaggccgtcatccctcacgcggcgtcgctgcatcaggctttcgcccattgtgcaatattccccactgctgcctcccgtaggagtctgggccgtgtctcagtcccagtgtggccggtcgccctctcaggccggctacccgtcgtcgccttggtaggccattaccccaccaacaagctgataggccgcgggctcatccttcaccgncggagctttaaccccgtcccatgcgggacagagtgttatccggtattagaacccgtttccagggcttgtcccagagtgaagggcagattgccacgtgttactcanccgttcgncactaatcancaacgaagcggcttcatcgttcgacttgcatgtgttaagcacgccgncagcgttcgtcctgagccaggatc

SEQ ID NO: 27 - DNA, 522 bp, actinomycete
  misc_feature (1)..(465): “n” is unknown nucleotide
tcagtatcngcccagagatccgccttcgccaccggtgtttcctcctgatatctgcgcatttcaccgctacaccaggaattccgatctcccctaccgaactctagcctgcccgtatcgactgcagacccggggttaagccccgggctttcacaaccgacgtgacaagccgcctacgagctctttacgcccaataattccggacaacgcttgcgccctacgtattaccgcggctgctggcacgtagttagccggcgcttcttctgcaggtaccgtcactttcgcttcttccctgctgaaagaggtttacaaaccgaaggccgtcatccctcacgcggcgtcgctgcatcaggctttcgcccattgtgcaatattccccactggtgnctcccgtangagtctggggcgtgtctcantccagtgtgggcggtcgcctctcagggcggctaccgtcgtcgcttggtgagncactactcacaacaagctgataggcgcgggctcatctggaacggcggagctttacac

SEQ ID NO: 28 - DNA, 670 bp, actinomycete
  misc_feature (1)..(638): “n” is unknown nucleotide
tcagtaatggcccaganatccgncttcgccaccggtgttcctcctgatatctgcgcatttcaccgctacaccaggaattccgatctcccctaccacactctaactagcccgtatcgaatgcagacccggggttaagccccgggctttcacatccgacgtgacaagccgcctacgagctctttacgcccaataattccggacaacgcttgcgccctacgtattaccgcggctgctggcacgtagttagccggcgcttcttctgcaggtaccgtcactttcgcttcttccctgctgaaagaggtttacaacccgaaggccgtcatccctcacgcggcgtcgctgcatcaggctttcgcccattgtgcaatattccccactgctgcctcccgtangagtctgggccgtgtctcagtcccagtgtggccggtcgccctctcaggccggctaccgtcgtcgccttggtaggccattacccaccaacaagctgatangncgngggctcatccttcaccgncggagctttcaancccgtcccatgcgggacagagtgttatccggtattaaacccgtntccagggcttgtccatagtgaagggcagattgccaagtgttatcanccgttcgncactaatcatcancgaagcggcttcatcgttcgactgcatgtgtt

SEQ ID NO: 29 - DNA, 676 bp, actinomycete
  misc_feature (1)..(666): “n” is unknown nucleotide
tcctcagnatcagtaatggcccagagatccgccttcgccaccggtgttcctcctgatatctgcgcatttcaccgctacaccaggaattccgatctcccctaccacactctanctagcccgtatcgaatgcagacccggggttaagccccgggctttcacatccgangtgacaagccgcctacgagctctttacgcccaataattccggacaangcttgcgccctacgtattaccgcggntgctggcacgtagttagccggcgcttcttctgcaggtaccgtcactttcgcttcttccctgctgaaagaggtttacaacccgaaggccgtcatccctcacncggcgtcgctgcatcaggctttcgcccattgtgcaatattccccactgctgcctcccgtaggagtctgggccgtgtctcaatcccantgtggccggtcgccctctcangccggctaccgtcgtcgcttggtaggccattaccccaccaacaagctggataggncgggggctcattcttcaccgccggaagctttaancccgtccatgcgggananagtgnatcccngtattaaacccngtttcagggcttgtccanagtgaagggngattgcccnagtgtttatcncccgttcgccantaatcnacaacgaaagcggnttcntcgnttcgacttgc

SEQ ID NO: 30 - DNA, 626 bp, actinomycete
  misc_feature (1)..(618): “n” is unknown nucleotide
taatggcccagaanatccgccttcgccaccggtgttcctcctgaatatctgcgcatttcaccgctacaccaggaattccgatctcccctaccacactctagctagcccgtatcgaatgcagacccggggttaagccccgggctttcacatccgacgtgacaagccgcctacgagctctttacgcccaataattccggacaacgcttgcgccctacgtattaccgcggctgctggcacgtagttagccggcgcttcttctgcaggtaccgtcactttcgcttcttccctgctgaaagaggtttacaacccgaaggccgtcatccctcacgcggcgtcgctgcatcaggctttcgcccattgtgcaatattccccactgctgcctcccgtaggagtctgggccgtgtctcagtcccagtgtggcggtcgccctctcaggccggntanccgtcgtcgccttggtangccattancccaccaacaagctgatangccgngggctcatccttcancgccggagcttttaaccccgtcccatgcgggacagagtgttatccggtattagatcccgtntccagggcttgtncatagtgaagggcanattgccacgtgttactcanccgttcgc

SEQ ID NO: 31 - DNA, 20 bp, primer
agagtttgatcmtggctcag

SEQ ID NO: 32 - DNA, 21 bp, primer
ctgtttgctccccacgctttc

SEQ ID NO: 33 - DNA, 22 bp, primer
tacggytaccttgttacgactt